(19) United States
(12) Patent Application Publication
(10) Pub. No.: US 2025/0319025 A9
(43) Pub. Date: Oct. 16, 2025

(54) COMPOSITIONS COMPRISING MODIFIED PHOSPHOLIPIDS AND USES THEREOF

(71) Applicant: The Children's Medical Center Corporation, Boston, MA (US)

(72) Inventors: Daniel S. Kohane, Newton, MA (US); Yang Li, Allston, MA (US)

(73) Assignee: The Children's Medical Center Corporation, Boston, MA (US)

(21) Appl. No.: 18/716,692

(22) PCT Filed: Dec. 6, 2022
(86) PCT No.: PCT/US2022/051905

§ 371 Date: Jun. 5, 2024

Related U.S. Application Data
(60) Provisional application No. 63/286,403, filed on Dec. 6, 2021.

(51) Int. Cl. A61K 9/127; A61K 31/137; A61K 31/445; A61K 31/529; A61K 31/573; A61K 31/704; A61K 47/24

(52) U.S. Cl. CPC A61K 9/1271; A61K 31/137; A61K 31/445; A61K 31/529; A61K 31/573; A61K 31/704; A61K 47/24

(57) ABSTRACT

Provided herein are compositions comprising compounds of Formula (I), and salts, co-crystals, tautomers, stereoisomers, solvates, hydrates, polymorphs, and isotopically enriched derivatives thereof; for example, in the form of a particle (e.g., liposome). Also provided are methods, uses, pharmaceutical compositions, and kits involving the compounds and/or compositions described herein, for methods for delivering an agent described herein (e.g., therapeutic agent, diagnostic agent), or for treating and/or preventing a disease in a subject, and methods of synthesizing these compositions.

### RELATED APPLICATIONS

This application claims priority under 35 U.S.C. § 119(e) to U.S. Provisional Application, U.S. Ser. No. 63/286,403, filed Dec. 6, 2021, which is incorporated herein by reference in its entirety.

### GOVERNMENT SUPPORT

This invention was made with government support under 1R35GM131728-01 awarded by the National Institutes of Health. The government has certain rights in the invention.

### BACKGROUND

Liposomes have been explored for drug delivery applications¹. Liposomal-based formulations have been approved for clinical use in treating various diseases including cancer, fungal infections and pain². Despite this success, some challenges remain, such as the inability to load sufficient cargo³, the difficulty in retaining some types of entrapped molecules in the liposome interior⁴, leakage of cargo and associated side effects⁵. Sufficient loading of drugs in the absence of a pH and ion gradient can be difficult⁶. Leakage from liposomes results in the release of significant amounts of entrapped payload immediately following administration (burst release), which may lead to unwanted local or systemic toxicity⁷. Leakage is particularly problematic for drugs with low molecular weight since the release of small molecules is much faster than large molecules⁸.

To address these challenges, efforts have been made to improve drug release kinetics from liposomes by altering lipid composition⁹. However, it usually requires preparing liposomes at higher temperature and many drugs like proteins may not survive the preparation procedures. Sustained drug release from liposomes has also been achieved by covalently conjugating reactive headgroups at the surface of lipid bilayers of multilamellar vesicles¹⁰. However, additional membrane fusion and covalent conjugation reactions make large scale production difficult, raise concern for quality assurance and cost, and hamper potential clinical translation⁹. Simple and effective engineering approaches or structural designs that could improve drug loading and control drug release from liposomes are still desired⁷.

Structurally, liposomes resemble biological lipid vesicles. In living organisms, natural lipids spontaneously form nanoscale to microscale vesicles, which maintain the cell and organelle integrity via creating physical barriers between the cells and subcellular compartments¹¹. The most abundant membrane lipids are the phospholipids that are composed of a polar head group and two acyl chains. In an aqueous environment, phospholipids self-assemble into lipid bilayer, in which the head groups face the surrounding water molecules and shield the interior hydrophobic acyl chains¹². Lipid bilayers are not static structures. The fluidity and mobility of lipid molecules allow given substances to pass through¹³. Movement across hydrophobic bilayers is the rate-limiting step in the passive diffusion of molecules through cell membranes because the interior hydrophobic phase is 100-1000 times more viscous than the surrounding aqueous phase¹⁴. The formation of such bilayers is driven by hydrophobic interactions, and van der Waals forces stabilize the packing of interior acyl chains⁹. The packing of the acyl chains influences the fluidity and permeability of lipid bilayers^{15,16}.

Insufficient drug loading and leakage of cargos remain major challenges in the design of liposome-based drug delivery systems. Leakage from liposomes results in the release of significant amounts of entrapped payload immediately following administration (burst release), which may lead to unwanted local or systemic toxicity. Leakage is particularly problematic for drugs with low molecular weight since the release of small molecules is much faster than large molecules. To achieve a strong therapeutic outcome, it is important that drugs are delivered to the disease site and become bioavailable at a level within their therapeutic window for a sufficient duration⁴. Drug delivery systems have been developed to improve the pharmacological properties and therapeutic efficacy of a broad range of drugs³⁸. Insufficient drug loading, leakage of entrapped payloads and associated sides effects remain major challenges in the design of liposome-based drug delivery systems⁷. Various chemical and engineering approaches have been developed to improve the drug loading and release in liposomes¹. However, direct chemical modifications and engineering of hydrophobic interior of lipid bilayers for drug delivery are largely unexplored.

Meanwhile, the quality of life of patients suffering from postoperative or even chronic pain is often diminished by the need for repeated administration of systemic analgesic medications (e.g., opioids), which give rise to potentially serious complications and clouding of the sensorium. Typically, repeated administration of systemic analgesic medications requires that patients be tethered to an external device, which can prolong hospitalization and even require that recipients be maintained as inpatients. Further, existing pain management options limit the ability of patients suffering from postoperative pain or chronic pain to adjust the timing, intensity and duration of anesthetic effect.

Peripheral nerves are surrounded by the perineurium, which is composed of a basal membrane with a layer of perineurial cells and tight junctions limiting paracellular permeability. Delivery of analgesic drugs is often impeded by the perineurium. For example, tetrodotoxin (TTX) is an attractive candidate in peripheral nerve anesthesia. It also does not cross the blood brain barrier. Voltage-gated sodium channels play important roles in nociceptive nerve conduction (Nassar M A, et al, Proc Natl Acad Sci USA, 101:12706-12711 (2004); Zimmermann K, et al, Nature, 447:855-858 (2007)). The efficacy of candidate anesthetics (e.g., specific antagonists of sodium channels) is impaired in vivo because of lack of permeability of the perineurial barrier. Hence, high concentrations of anesthetics and multiple dosages are often required to achieve clinically effective and prolonged anesthesia. Although permeation enhancers have been used to increase the permeability of lipid barriers and, they can be associated with myotoxicity. There exists a need for systems for the delivery of local anesthetics which provide repeated or prolonged analgesia on-demand, following a single administration of the anesthetics.

There is therefore a need for compositions and methods for the synthesis of new phospholipids with chemical modifications that can alter the permeability of liposomes. There is also a need for a liposome-based controlled delivery system for which the drug release can be modulated by incorporated functional groups within lipid bilayers. There is a need for specific formulations of local anesthetics which are both safe and efficacious in humans, that elicit prolonged peripheral nerve blockade for up to three or more days following a single application, as well as a need for specific formulations of different classes of drugs (e.g., two or more different classes of drugs) and a trigger release system which elicit on-demand, repeatable, adjustable peripheral nerve blockade following a single injection.

Phospholipids in which functional groups are covalently conjugated to the acyl chains could address these needs. In one aspect, formulations of acyl-chain modified phospholipids and/or of natural phospholipids can afford liposome formulations, resulting in a change in loading efficiency and release kinetics of payload. In some embodiments, these formulations could be used to deliver payload agents in therapeutic and/or diagnostic methods, for example, for local anesthesia, photodynamic therapy, inflammation, molecular imaging, photothermal therapy, and/or fluorescence imaging.

### SUMMARY

Disclosed herein are compositions comprising phospholipids (e.g., phospholipids optionally comprising modifications on the acyl chains), and methods of synthesis and uses thereof. In some embodiments, the phospholipids described herein comprise a hydrophilic head group and a hydrophobic acyl tail, wherein terminal aromatic groups are covalently conjugated to the acyl tails to render altered liposomal permeability. The phospholipids spontaneously form liposomes upon hydration.

Also described herein are compositions and methods for the formulation of liposomes with phospholipids (e.g., comprising phospholipids with modified acyl chains). The new synthetic phospholipids can be used for the delivery of a broad range of therapeutics, for example, for delivering agents for prolonged nerve blockade with local anesthetics. In the aromatized liposomes described herein show similar morphology to conventional liposomes. Aromatic groups may decrease the permeability of lipid bilayers, which provide additional stabilization forces for tight packing of acyl chains. Aromatized liposomes described herein appear to enable increased drug loading, prolonged therapeutic duration, expanded therapeutic window, and mitigate systemic toxicity of anesthetic drugs with low molecular weight, extremely high potency and a narrow therapeutic window. The rationally designed liposomes therefore create a new paradigm for the delivery of a broad range of therapeutic agents that otherwise might not be clinically applicable.

In one aspect, disclosed herein is a composition comprising: a compound of Formula (I):

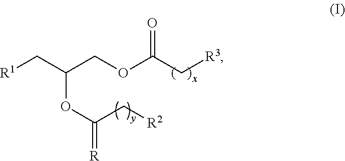

or a salt, co-crystal, tautomer, stereoisomer, solvate, hydrate, polymorph, or an isotopically enriched derivative thereof, wherein:
x is 1, 2, 3, 4, 5, 6, 7, 8, 9, 10, 11, 12, 13, 14, 15, 16, 17, 18, 19, 20, 21, 22, 23, or 24;
y is 1, 2, 3, 4, 5, 6, 7, 8, 9, 10, 11, 12, 13, 14, 15, 16, 17, 18, 19, 20, 21, 22, 23, or 24;
R¹ is a phosphoglycerol, phosphocholine, phosphoethanolamine, phosphoinositol, or phosphoserine moiety;
R² is halogen, optionally substituted acyl, optionally substituted branched or unbranched alkyl, optionally substituted carbocyclyl, optionally substituted heterocyclyl, optionally substituted aryl, —OR^{D1}, —N(R^{D1a})₂, —SR^{D1}, —SO₂, —NO₂, —N₃, or —CN;
R³ is halogen, optionally substituted acyl, optionally substituted branched or unbranched alkyl, optionally substituted carbocyclyl, optionally substituted heterocyclyl, optionally substituted aryl, —OR^{D1}, —N(R^{D1a})₂, —SR^{D1}, —SO₂, —NO₂, —N₃, or —CN;
wherein R^{D1} is independently hydrogen, optionally substituted acyl, optionally substituted alkyl, optionally substituted alkenyl, optionally substituted cycloalkynyl group, optionally substituted carbocyclyl, optionally substituted heterocyclyl, optionally substituted aryl, optionally substituted heteroaryl, or an oxygen protecting group when attached to an oxygen atom, or a sulfur protecting group when attached to a sulfur atom; and
wherein each occurrence of R^{D1a} is independently hydrogen, optionally substituted acyl, optionally substituted alkyl, optionally substituted alkenyl, optionally substituted linear alkynyl, optionally substituted cycloalkynyl group, optionally substituted carbocyclyl, optionally substituted heterocyclyl, optionally substituted aryl, optionally substituted heteroaryl, or a nitrogen protecting group; or optionally two instances of R^{D1a} are taken together with their intervening atoms to form a substituted or unsubstituted heterocyclic or substituted or unsubstituted heteroaryl ring. In certain embodiments, the compound is of Formula (I-A):

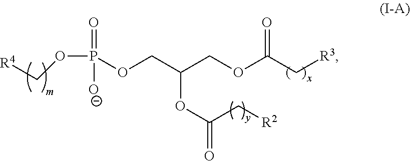

or a salt, solvate, hydrate, stereoisomer, polymorph, tautomer, isotopically enriched form, or prodrug thereof, wherein:
R⁴ is

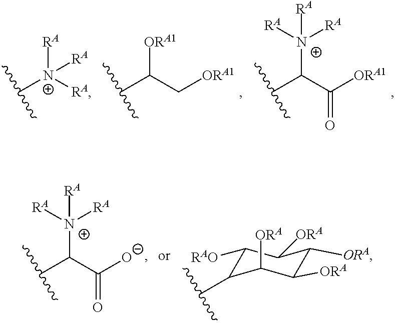

as valency permits;
each occurrence of R^{A} is independently hydrogen or unsubstituted alkyl;
each occurrence of R^{A} is independently hydrogen, unsubstituted alkyl, or an oxygen protecting group; and
m is 1, 2, 3, 4, 5, or 6.

In certain embodiments, the compound is of Formula (I-A-1):

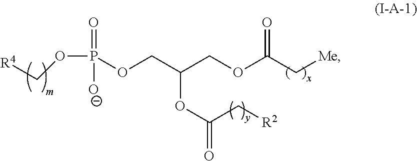

or a salt, solvate, hydrate, stereoisomer, polymorph, tautomer, isotopically enriched form, or prodrug thereof, wherein:
R⁴ is

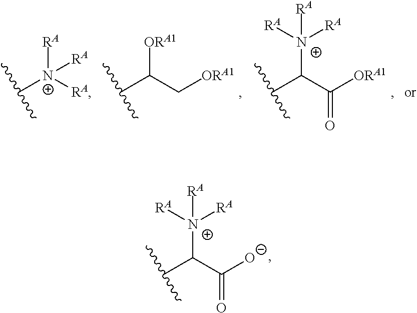

as valency permits;
each occurrence of R^{A} is independently hydrogen or unsubstituted alkyl;
each occurrence of R^{A1} is independently hydrogen, unsubstituted alkyl, or an oxygen protecting group; and
m is 1, 2, 3, 4, 5, or 6. In certain embodiments, R¹ is an unsubstituted phosphoglycerol, unsubstituted phosphocholine, unsubstituted phosphoethanolamine, unsubstituted phosphoinositol, or unsubstituted phosphoserine moiety. In certain embodiments, R⁴ is

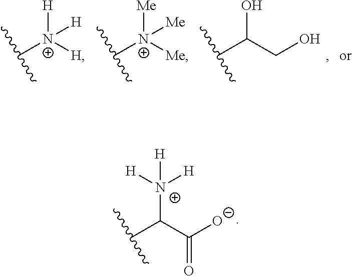

In certain embodiments, R⁴ is

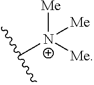 [SMILES: *[N+](C)(C)C]

In certain embodiments, m is 2. In certain embodiments, x is 12, 13, 14, 15, or 16. In certain embodiments, x is 14. In certain embodiments, x is 15. In certain embodiments, y is 15. In certain embodiments, R² is —OR^{D1}, and R^{D1} is optionally substituted acyl, optionally substituted aryl, optionally substituted heteroaryl, optionally substituted cycloalkynyl group, or optionally substituted linear alkynyl. In certain embodiments, R³ is —OR^{D1}, and R^{D1} is optionally substituted acyl, optionally substituted aryl, optionally substituted heteroaryl, optionally substituted cycloalkynyl group, or optionally substituted linear alkynyl. In certain embodiments, R³ is methyl and R² is —OR^{D1}, and R^{D1} is optionally substituted acyl, optionally substituted aryl, optionally substituted heteroaryl, optionally substituted cycloalkynyl group, or optionally substituted linear alkynyl.

In certain embodiments, R^{D1} is

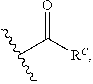

optionally substituted linear alkynyl, optionally substituted phenyl, optionally substituted naphthyl, optionally substituted anthryl, optionally substituted 2H-chromen-2-one (coumarin), optionally substituted cyclooctynyl, optionally substituted dibenzocyclooctyne, or optionally substituted aza-dibenzocyclooctyne; and
R^{C} is hydrogen, optionally substituted alkyl, or optionally substituted alkenyl. In certain embodiments, R² is of the formula:

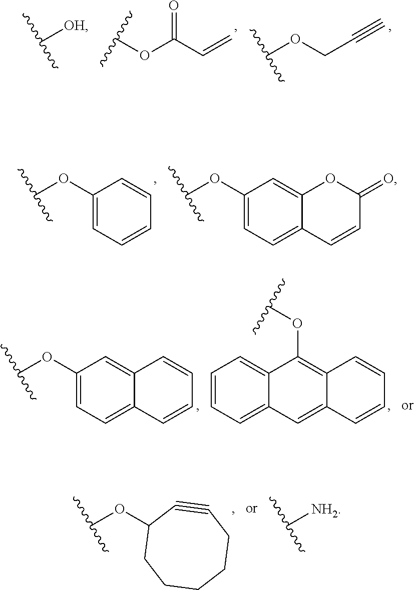

In certain embodiments, R² is —N(R^{D1a})₂, —N(R^{D1a})C(═O)OR^{D1}, or —SR^{D1};
R^{D1} is hydrogen, optionally substituted alkyl, optionally substituted aryl, optionally substituted heteroaryl, or optionally substituted cycloalkynyl group; and
   each instance of R^{D1a} is independently hydrogen, optionally substituted acyl, optionally substituted alkyl, or optionally substituted alkenyl. In certain embodiments, R² is of the formula:

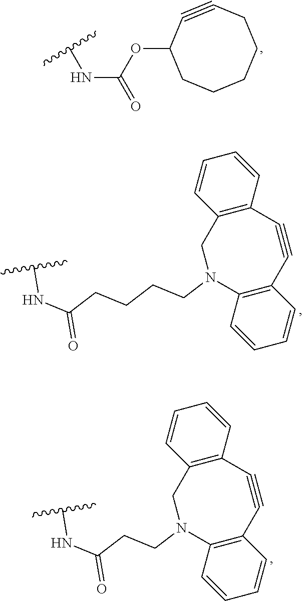 [SMILES: *NC(=O)OC1C#CCCCCC1 ; *NC(=O)CCCCN1Cc2ccccc2C#Cc2ccccc21 ; *NC(=O)CCN1Cc2ccccc2C#Cc2ccccc21]

or —SH. In certain embodiments, R² is —Br or —N₃. In certain embodiments, R² is

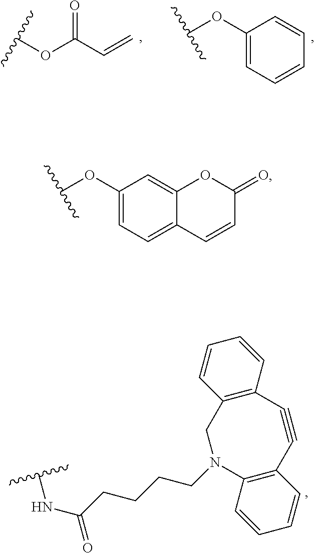

—SH, or —N₃. In certain embodiments, R² is

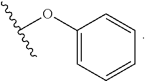 [SMILES: *Oc1ccccc1]

In certain embodiments, the compound is of formula:

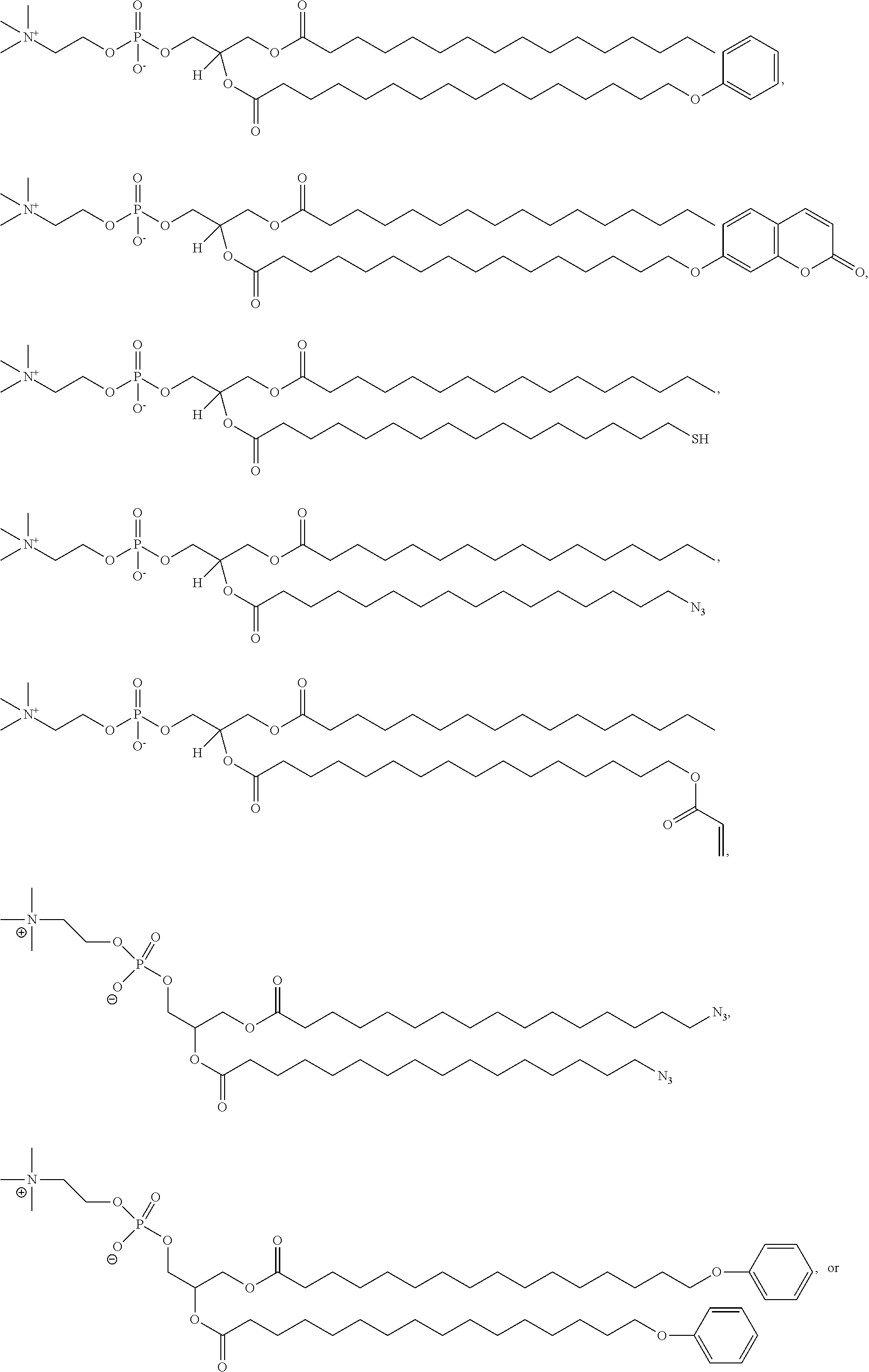

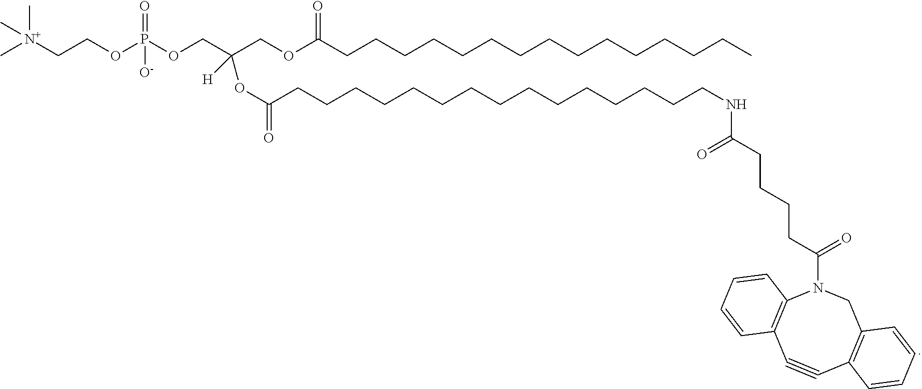 [SMILES: CCCCCCCCCCCCCCCC(=O)OCC(COP(=O)([O-])OCC[N+](C)(C)C)OC(=O)CCCCCCCCCCCCCCCNC(=O)CCCCC(=O)N1Cc2ccccc2C#Cc2ccccc21]

In certain embodiments, the composition comprises non-phosphorous containing lipids such as, but not limited to, stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyloxylated fatty acid amides, dioctadecyldimethyl ammonium bromide and the like, diacylphosphatidylcholine, diacylphosphatidylethanolamine, ceramide, sphingomyelin, cephalin, and cerebrosides. Lipids such as lysophosphatidylcholine and lysophosphatidylethanolamine may be used in some instances. In certain embodiments, the composition further comprises polyethylene glycol-based polymers such as, but not limited to, PEG 2000, PEG 5000 and polyethylene glycol conjugated to phospholipids or to ceramides (referred to as PEG-Cer). In some instances, modified forms of lipids may be used including forms modified with detectable labels such as fluorophores. In some instances, the lipid is a lipid analog that emits signal (e.g., a fluorescent signal). In some instances, the lipid is a lipid analog that comprises a fluorophore, such as, but not limited to, sulforhodamine B, indocyanine green, methylene blue, or coumarin. In some instances, the lipid is a lipid analog that comprises sulforhodamine B or indocyanine green.

In certain embodiments, the composition further comprises one or more agents. In certain embodiments, at least one of the one or more agents is a therapeutic agent or diagnostic agent. In certain embodiments, the therapeutic agent is an antibiotic agent, chemotherapeutic agent, anesthetic agent, anti-inflammatory agent, analgesic agent, anti-fibrotic agent, anti-sclerotic agent, or anticoagulant agent. In certain embodiments, the therapeutic agent is a local anesthetic. In certain embodiments, the local anesthetic is a sodium channel blocker, for example, a site 1 sodium channel blocker, amino ester local anesthetic, or an amino amide local anesthetic. In certain embodiments, the therapeutic agent is a chemotherapeutic agent or an antibiotic agent. In certain embodiments, the therapeutic agent is an anesthetic agent, chemotherapeutic agent, or an antibiotic agent. In certain embodiments, the therapeutic agent is an anesthetic agent or a chemotherapeutic agent. In certain embodiments, the therapeutic agent is doxorubicin, tetrodotoxin, saxitoxin, neosaxitoxin, bupivacaine, amylocaine, ambucaine, articaine, benzocaine, benzonatate, butacaine, butanilicaine, carbocaine, cepastat, chloraseptic, chloroprocaine, cinchocaine, citanest, cyclomethycaine, dibucaine, diperodon, dimethocaine, eucaine, etidocaine, fomocaine, fotocaine, hydroxyprocaine, isobucaine, levobupivacaine, lidocaine, marcaine, mepivacaine, meprylcaine, metabutoxycaine, nitracaine, orthocaine, orabloc, oxetacaine, oxybuprocaine, paraethoxycaine, phenacaine, piperocaine, piridocaine, polocaine, posimir, pramocaine, prilocaine, primacaine, procaine, procainamide, proparacaine, propoxycaine, pyrrocaine, quinisocaine, ropivacaine, sensorcaine, septocaine, trimecaine, tetracaine, tolycaine, tropacocaine, ulcerease, xylocaine, or zorcaine. In certain embodiments, the agent is doxorubicin, bupivacaine, or tetrodotoxin. In certain embodiments, the agent is doxorubicin. In certain embodiments, the agent is bupivacaine or tetrodotoxin. In certain embodiments, the agent is bupivacaine. In certain embodiments, the agent is tetrodotoxin. In certain embodiments, the agent is a small molecule therapeutic agent, a protein therapeutic agent, a nucleic acid therapeutic agent, or small molecule diagnostic agent. In certain embodiments, the diagnostic agent is a fluorophore. In certain embodiments, the diagnostic agent is conjugated to a protein, a polymer, or a small molecule. In certain embodiments, the diagnostic agent is Sulforhodamine B, indocyanine green, fluorescein isothiocyanate, methylene blue, or coumarin. In certain embodiments, the composition is in the form of a particle. In certain embodiments, the particle has an average diameter of approximately 0.5-2 μm, approximately 0.5-1.0 μm, approximately 1-1.5 μm, approximately 1.5-2 μm, approximately 2-2.5 μm, approximately 1-2 μm, or approximately 2-3 μm. In certain embodiments, the particle has an average diameter of approximately 0.5-2 μm (e.g., approximately 1 μm, 1 μm). In certain embodiments, the particle has an average zeta potential of approximately −35-50 mV (e.g., approximately −30 mV). In certain embodiments, the particle has an average polydispersity value of approximately 0.1-0.2 (e.g., 0.15). In certain embodiments, the particle has an average diameter of approximately 0.5-2 μm (e.g., approximately 1 μm, 1 μm), average zeta potential of approximately −35-50 mV (e.g., approximately −30 mV), and an average polydispersity value of approximately 0.1-0.2 (e.g., 0.15). In certain embodiments, the particle is a liposome, lipid nanoparticle, polymer-lipid hybrid nanoparticle, or lipid coated inorganic nanoparticle.

In another aspect, disclosed herein is a pharmaceutical composition comprising a therapeutic agent, and optionally a pharmaceutically acceptable excipient. In another aspect, disclosed herein are methods of delivering an agent to a subject or biological sample, comprising administering to the subject or contacting the biological sample with a composition described herein, or administering to the subject or contacting the biological sample with the pharmaceutical composition described herein. In another aspect, disclosed herein are methods of treating and/or preventing a disease in a subject in need thereof, the method comprising administering to the subject a composition described herein comprising a therapeutically effective amount of a therapeutic agent, or a pharmaceutical composition described herein. In another aspect, disclosed herein is use of a composition delivering an agent to a subject, the use comprising administering to the subject a composition described herein. In another aspect, disclosed herein is use of a composition to treat and/or prevent a disease in a subject in need thereof, the use comprising administering to the subject a composition described herein comprising a therapeutically effective amount of a therapeutic agent, or a pharmaceutical composition described herein.

In another aspect, disclosed herein is a kit for delivering an agent to a subject, comprising a composition described herein, the agent, and instructions for delivering the agent to a subject in need thereof.

The present application refers to various issued patents, published patent applications, journal articles, and other publications, all of which are incorporated herein by reference. The details of one or more embodiments of the invention are set forth herein. Other features, objects, and advantages of the invention will be apparent from the Detailed Description, Examples, Figures, and Claims.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer, or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

When a range of values is listed, it is intended to encompass each value and sub-range within the range. For example, “C₁₋₆” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆. For example, “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “alkyl” refers to a radical of a straight-chain or branched saturated hydrocarbon group having from 1 to 20 carbon atoms (“C₁₋₂₀ alkyl”). The term “branched alkyl” refers to a radical of a branched saturated hydrocarbon group having from 1 to 20 carbon atoms (“branched C₁₋₂₀ alkyl”), for example, isopropyl, t-butyl, sec-butyl, iso-butyl, neopentyl, isopentyl, and neoheptyl. The term “unbranched alkyl” is the same as a straight-chain or linear alkyl group, i.e., an alkyl group having no alkyl branching groups. In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

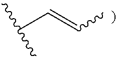

may be in the (E)- or (Z)-configuration.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). The term “linear alkynyl” refers to a radical of a straight-chain, unbranched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl. In certain embodiments, the alkynyl group is an optionally substituted C₂₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₆ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃_cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl, and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetra-hydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 □ electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{bb})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{cc})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, =NNR^{bb}C(═O)OR^{aa}, =NNR^{bb}S(═O)₂R^{aa}, =NR^{bb}, or =NOR^{cc};
wherein:
each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or =S; wherein X⁻ is a counterion;
each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C)C(═O)N, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R^{gg} substituents can be joined to form ═O or ═S; and
each X⁻ is a counterion.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, the molecular weight of a carbon atom substituent is lower than 250, lower than 200, lower than 150, lower than 100, or lower than 50 g/mol. In certain embodiments, a carbon atom substituent consists of carbon, hydrogen, fluorine, chlorine, bromine, iodine, oxygen, sulfur, nitrogen, and/or silicon atoms. In certain embodiments, a carbon atom substituent consists of carbon, hydrogen, fluorine, chlorine, bromine, iodine, oxygen, sulfur, and/or nitrogen atoms. In certain embodiments, a carbon atom substituent consists of carbon, hydrogen, fluorine, chlorine, bromine, and/or iodine atoms. In certain embodiments, a carbon atom substituent consists of carbon, hydrogen, fluorine, and/or chlorine atoms.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{aa})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═S)OR^{aa}, —SC(═S) N(R^{bb})₂, —SC(═O)SR^{aa}, —SC(═O)OR^{aa}, —SC(═O)N(R^{bb})₂, and —SC(═O)R^{aa}, wherein R^{aa} and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

The term “silyl” refers to the group —Si(R^{aa})₃, wherein R^{aa} is as defined herein.

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that include the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3′″-[N-(imidazolylmethyl)]trityl Ether (IDTr-OR), 4,4′-Dimethoxy-3′″-[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). In some embodiments, each sulfur protecting group is selected from the group consisting of —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]—, B(C₆F₅)₄, BPh₄, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “stoichiometric solvate” refers to a solvate, which comprises a compound (e.g., a compound disclosed herein) and a solvent, wherein the solvent molecules are an integral part of the crystal lattice, in which they interact strongly with the compound and each other. The removal of the solvent molecules will cause instability of the crystal network, which subsequently collapses into an amorphous phase or recrystallizes as a new crystalline form with reduced solvent content.

The term “non-stoichiometric solvate” refers to a solvate, which comprises a compound (e.g., a compound disclosed herein) and a solvent, wherein the solvent content may vary without major changes in the crystal structure. The amount of solvent in the crystal lattice only depends on the partial pressure of solvent in the surrounding atmosphere. In the fully solvated state, non-stoichiometric solvates may, but not necessarily have to, show an integer molar ratio of solvent to the compound. During drying of a non-stoichiometric solvate, a portion of the solvent may be removed without significantly disturbing the crystal network, and the resulting solvate can subsequently be resolvated to give the initial crystalline form. Unlike stoichiometric solvates, the desolvation and resolvation of non-stoichiometric solvates is not accompanied by a phase transition, and all solvation states represent the same crystal form.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “crystalline” or “crystalline form” refers to a solid form substantially exhibiting three-dimensional order. In certain embodiments, a crystalline form of a solid is a solid form that is substantially not amorphous. In certain embodiments, the X-ray powder diffraction (XRPD) pattern of a crystalline form includes one or more sharply defined peaks.

The term “co-crystal” refers to a crystalline structure comprising at least two different components (e.g., a compound disclosed herein and an acid), wherein each of the components is independently an atom, ion, or molecule. In certain embodiments, none of the components is a solvent. In certain embodiments, at least one of the components is a solvent. A co-crystal of a compound disclosed herein and an acid is different from a salt formed from a compound disclosed herein and the acid. In the salt, a compound disclosed herein is complexed with the acid in a way that proton transfer (e.g., a complete proton transfer) from the acid to a compound disclosed herein easily occurs at room temperature. In the co-crystal, however, a compound disclosed herein is complexed with the acid in a way that proton transfer from the acid to a compound disclosed herein does not easily occur at room temperature. In certain embodiments, in the co-crystal, there is no proton transfer from the acid to a compound disclosed herein. In certain embodiments, in the co-crystal, there is partial proton transfer from the acid to a compound disclosed herein. Co-crystals may be useful to improve the properties (e.g., solubility, stability, and ease of formulation) of a compound disclosed herein.

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refers to compounds, including derivatives of the compounds described herein, that have cleavable groups and become by solvolysis or under physiological conditions the compounds described, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁-C₈ alkyl, C₂-C₈ alkenyl, C₂-C₈ alkynyl, aryl, C₇-C₁₂ substituted aryl, and C₇-C₁₂ arylalkyl esters of the compounds described herein may be preferred.

The term “small molecule” refers to molecules, whether naturally occurring or artificially created (e.g., via chemical synthesis) that have a relatively low molecular weight. Typically, a small molecule is an organic compound (e.g., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present invention.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development. A non-human animal may be a transgenic animal.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments, organelles (such as obtained by lysing cells and separating the components thereof by centrifugation or otherwise). Other examples of biological samples include blood, serum, urine, semen, fecal matter, cerebrospinal fluid, interstitial fluid, mucus, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample. Biological samples also include those biological samples that are transgenic, such as a transgenic oocyte, sperm cell, blastocyst, embryo, fetus, donor cell, or cell nucleus, or cells or cell lines derived from biological samples.

The term “tissue” refers to any biological tissue of a subject (including a group of cells, a body part, or an organ) or a part thereof, including blood and/or lymph vessels, which is the object to which a compound, particle, and/or composition of the invention is delivered. A tissue may be an abnormal or unhealthy tissue, which may need to be treated. A tissue may also be a normal or healthy tissue that is under a higher than normal risk of becoming abnormal or unhealthy, which may need to be prevented. In certain embodiments, the tissue is the central nervous system. In certain embodiments, the tissue is the brain.

The terms “administer,” “administering,” or “administration” refer to implanting, absorbing, ingesting, injecting, inhaling, or otherwise introducing a compound, or a pharmaceutical composition thereof.

The terms “treatment,” “treat,” and “treating” refer to reversing, alleviating, delaying the onset of, or inhibiting the progress of a “pathological condition” (e.g., a disease, disorder, or condition, or one or more signs or symptoms thereof) described herein. In some embodiments, treatment may be administered after one or more signs or symptoms have developed or have been observed. In other embodiments, treatment may be administered in the absence of signs or symptoms of the disease or condition. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response, i.e., treating the condition. As will be appreciated by those of ordinary skill in this art, the effective amount of a compound described herein may vary depending on such factors as the desired biological endpoint, the pharmacokinetics of the compound, the condition being treated, the mode of administration, and the age and health of the subject. An effective amount encompasses therapeutic and prophylactic treatment. For example, in treating cancer, an effective amount of a compound may reduce the tumor burden or stop the growth or spread of a tumor.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces, or avoids symptoms or causes of the condition, or enhances the therapeutic efficacy of another therapeutic agent.

A “prophylactically effective amount” of a compound described herein is an amount sufficient to prevent a condition, or one or more signs or symptoms associated with the condition, or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

### BRIEF DESCRIPTION OF THE DRAWINGS

FIG. 1 shows a schematic of liposomes prepared with acyl chain-modified phospholipids.

FIG. 2 shows the representative synthesis of acyl chain-modified phospholipids.

FIG. 3 shows the ¹H-NMR spectrum of methyl 16-bromohexadecanoate.

FIG. 4 shows the ¹H-NMR spectrum of methyl 16-phenoxyhexadecanoate.

FIG. 5 shows the ¹H-NMR spectrum of 16-phenoxyhexadecanoic acid.

FIG. 6 shows the ¹H-NMR spectrum of 1-palmitoyl-2-(16-phenoxy)palmitoyl-sn-glycero-3-phosphocholine (Ph-DPPC). Peaks as a result of phenoxy-conjugation are highlighted.

FIG. 7 shows the ¹H-NMR spectrum of 1-palmitoyl-2-(16-coumarin)palmitoyl-sn-glycero-3-phosphocholine (CM-DPPC). Peaks as a result of coumarin-conjugation were highlighted.

FIG. 8 shows the mass spectra of the depicted acyl chain-modified phospholipids.

FIG. 9 shows the phase transition temperature of acyl chain-modified phospholipids.

FIGS. 10A-10B show dynamic light scattering and a TEM image of aromatized liposomes (“Lip-Ph”—which are liposomes containing the synthetic aromatized phospholipid Ph-DPPC). The structures of the Lip-Ph which forms liposomes and NMR characterization thereof are shown in FIG. 6. FIG. 10C is a graph showing the stability of the aromatized liposomes. FIG. 10D is a photograph of a solution of the aromatized liposomes. FIG. 10E is a graph showing the size of the aromatized liposomes after extrusion.

FIGS. 11A-11H show the encapsulation and cumulative release of Sulforhodamine B (SRho). Data are means±SD, n=4 independent experiments. FIG. 11A shows the chemical structure of sulforhodamine B. FIG. 11B is a graph showing SRho loading in different chemically modified liposomes. FIG. 11C is a graph showing cumulative release of SRho from different formulations at 37° C. (p-values compare groups at 168 h). FIG. 11D is a graph showing the viscosity of different liposomal formulations. FIG. 11E shows the chemical structure of DBCO-modified phospholipids (DBCO-DPPC) and mass spectra of CBO-DPPC. The DBCO group was covalently conjugated to the acyl chain of phospholipid via polar amide bonds. FIG. 11F is a graph showing loading in DBCO-modified liposomes (SRho@Lipo-DBCO). FIG. 11G is a graph showing cumulative release of SRho from SRho@Lipo-DBCO (p-values compare groups at 168 h). FIG. 11H is a graph showing cumulative release of SRho from different formulations. Ph+SRho@Lipo and ICG+SRho@Lipo are liposomes containing physically encapsulated phenol or indocyanine green (ICG). SRho@Lipo-Ph are liposomes containing covalently conjugated phenoxy group (p-values compare groups at 168 h).

FIGS. 11I-11J are graphs showing the cytotoxicity of aromatic molecules. FIG. 11I shows cell viability of C2C12 cells incubated with free aromatic molecules and aromatic molecule-loaded liposomes; and FIG. 11J shows cell viability of PC12 cells incubated with free aromatic molecules and aromatic molecule-loaded liposomes. The concentration of Phenol and ICG were 5 mM. Data are means±s.d., n=4.

FIG. 12 is a graph showing pH-dependent partition coefficients of different payloads. Octanol-water partition coefficients were quantified using a miniaturized shake-flask approach.

FIG. 13A shows the structures of tetrodotoxin, bupivacaine, and doxorubicin.

FIGS. 13B-13E show the effect of liposome aromatization on the loading of different payloads (tetrodotoxin, bupivacaine, doxorubicin, rhodamine, PEG, albumin). FIG. 13B-C are graphs showing the increase of small molecule loading as a function of molecular weight and hydrophilicity. FIG. 13D-E are graphs showing the increase of macromolecule loading as a function of molecular weight and hydrophilicity.

FIGS. 13F-13H are graphs showing the cumulative release of tetrodotoxin (TTX, FIG. 13F), bupivacaine hydrochloride (Bup, FIG. 13G), and doxorubicin hydrochloride (Dox, FIG. 13H).

FIGS. 13I-13J are graphs showing the reduction of the release of small molecular payloads as the function of molecular weight (FIG. 13I) and hydrophilicity (FIG. 13J).

FIGS. 13K-13M are graphs showing cumulative release of rhodamine-conjugated polyethylene glycol with a molecular weight of 1,000 (SRho-PEG1k, FIG. 13K), rhodamine-conjugated polyethylene glycol with a molecular weight of 10,000 (SRho-PEG10k, FIG. 13L), and albumin-fluorescein isothiocyanate conjugate (FITC-Ab, FIG. 13M).

FIGS. 13N-13O are graphs showing the reduction of drug release as the function of molecular weight (FIG. 13N) and hydrophilicity (FIG. 13O).

FIG. 13P is a graph showing cumulative TTX release from different formulations at 37° C. TTX concentrations were quantified by TTX ELISA. Data are means±s.d., n=4. p-values compare groups at 168 h.

FIGS. 14A-14H show physiochemical characterizations of lipid bilayer-engineered vesicles. FIGS. 14A and 14B show number-weighted diameters of indicated liposomes (liposome (Lip); unmodified liposome with TTX (Lip-TTX); aromatized liposomes encapsulating TTX (Lip-Ph-TTX)) measured by dynamic light scattering. FIG. 14C shows transmission electron micrograph images of liposomes. Scale bar, 600 nm. FIG. 14D shows zeta potential of liposomes. Data are mean s.d., n=4. FIG. 14E, Viscosity of liposomal formulations as a function of shear rate. FIGS. 14F and 14G show cytotoxicity of C2C12 cells (FIG. 14F) and PC12 (FIG. 14G) cells incubated with different formulations. Data are mean s.d., n=4. FIG. 13H shows loading efficiency of tetrodotoxin quantified by enzyme-linked immunosorbent assay (ELISA). Data are mean s.d., n=4.

FIGS. 15A-15B show cytotoxicity of C2C12 cells and PC12 cells incubated with different formulations (PBS, free TTX, Lip-TTX, Lip-Ph-TTX). Different formulations were directly added into the cell culture media. Data are means±SD, n=4.

FIG. 16 shows the cumulative release of free TTX as quantified by enzyme-linked immunosorbent assay. Data are means±SD, n=4 independent experiments.

FIGS. 17A-17B show the retention and localization of the indicated liposomal formulations. FIG. 17A, Representative whole-body image of rats injected with different Cy7-labeled formulations. Fluorescence intensity is represented as radiant efficiency. FIG. 17B, Representative fluorescent confocal photomicrographs 24 hours after administration of different Cy7-labeled formulations, with corresponding hematoxylin-eosin stained sections. The dotted line indicates the nerve perimeter. In FIG. 17 and throughout the Figures, “Lip” refers to conventional liposomes containing only natural phospholipids (DPPC). Cy7-Lip refers to conventional liposomes with covalently conjugated Cy7 dye. Cy7-Lip-Ph refers to aromatized liposomes with covalently conjugated Cy7 dye.

FIG. 18 shows the representative whole-body image of rats injected with free Cy7. Fluorescence intensity is represented as radiant efficiency.

FIG. 19 shows representative fluorescent confocal photomicrographs 0.5 hours after administration of free Cy7, with corresponding hematoxylin-eosin-stained sections. The dotted line indicates the nerve perimeter.

FIG. 20 is a graph showing quantification of the fluorescence intensity over time (as a percentage of fluorescence at time=0, immediately after injection) for Cy7-conjugated liposomes and free Cy7.

FIGS. 21A-21H show in vivo performance of TTX formulations. FIG. 19A shows schematic of tetrodotoxin (TTX)-encapsulated liposomes for prolonged duration local anesthesia following sciatic nerve injection. Upon release, TTX blocks the flow of Na+ through voltage-gated sodium channels, thereby providing nerve block. FIG. 21B shows representative time courses of sciatic nerve block following injection of different TTX formulations. FIG. 21C shows duration of sensory nerve blockade of different TTX formulations. Data are means±s.d.; n=8 for each group. FIG. 21D shows duration of sensory nerve blockade from different formulations injected at the sciatic nerve. Daggers indicate 100% mortality. Data are means±s.d. n≥4. p-values are from unpaired two-tailed t-test. NS, P>0.05 comparing TTX@Lipo at 24.8 μg TTX and ICG+TTX@Lipo at 24.4 μg; **P=0.007 comparing ICG+TTX@Lipo at 31.0 μg TTX and TTX@Lipo-Ph at 32.1 μg TTX. FIG. 21E shows thermal latency in the un-injected (contralateral) extremity in the first 12 hours after injection. Data are presented as mean s.d.; n=8 for each group. FIG. 21F shows frequency of block in the contralateral (uninjected) leg. FIG. 21G shows mortality from TTX formulations. FIG. 21H is a graph showing the effect of epinephrine and dexamethasone on the duration of nerve block from TTX@Lipo-Ph.

FIG. 22 shows a comparison of the durations of sensory and motor blocks of different TTX formulations. Data are means±s.d., n=4. The diagonal dotted line denotes equal durations of sensory and motor block.

FIGS. 23A-23B show the thermal latency in the un-injected (contralateral) extremity after injection. (FIG. 23A) Lip-Ph-TTX; (FIG. 21B) Lip-Ph-ICG-TTX.

FIG. 24 shows temperatures of a solution of Lip-Ph-ICG over time (minutes) with continuous irradiation of near infrared laser at different intensity.

FIG. 25 shows photo-triggered local anesthesia in the rat footpad. Following injection of 100 of Lip-Ph-ICG-TTX and subsequent irradiation (arrows, 808 nm continuous wave NIR laser at 200 mW/cm2 for 5 minutes), the effect of local anesthesia is represented as a percentage of maximum possible effect.

FIGS. 26A-26B show the cytotoxicity of formulations to C2C12 (24A) and PC12 (24B) cells. Data are means±s.d., n=4.

FIGS. 27A-27B show the tissue reaction to TTX-encapsulated liposomes, in rats injected with the indicated TTX formulations (Lip-TTX, Lip-ICG-TTX, Lip-Ph-TTX, and Lip-Ph-ICG-TTX). FIG. 27A: Representative photographs of the site of injection upon dissection 4 days after injection. FIG. 27B: Representative H&E-stained sections of nerves and surrounding tissues, and toluidine blue-stained section of nerves. The scale bar for H&E-stained sections is 100 μm; for toluidine blue is 20 μm.

FIG. 28 shows representative photographs of the site of injection upon dissection 14 days after injection of different liposome-TTX formulations, in the rats of FIG. 27. The green color is due to ICG.

FIG. 29 shows toluidine blue-stained sections of sciatic nerves 4 days and 14 days after injection of different TTX formulations, in the rats of FIG. 27. Scale bar: 100 μm.

### DETAILED DESCRIPTION OF CERTAIN EMBODIMENTS OF THE INVENTION

The present invention provides compositions, which form into particles (e.g., liposomes, lipid nanoparticles, polymer-lipid hybrid nanoparticles, lipid coated inorganic nanoparticles), wherein the composition comprises a compound of Formula (I), or a salt, co-crystal, tautomer, stereoisomer, solvate, hydrate, polymorph, or an isotopically enriched derivative thereof, and methods of use (e.g., delivering therapeutic agents, diagnostic agents) to a subject or biological sample; methods of treating and/or preventing a disease in a subject in need thereof, the method comprising administering to the subject a composition described herein; pharmaceutical compositions comprising the compound of Formula (I), or a salt, co-crystal, tautomer, stereoisomer, solvate, hydrate, polymorph, or an isotopically enriched derivative thereof and an agent (e.g., therapeutic agents, diagnostic agents); and uses thereof.

In certain embodiments, the compound is of Formula (I):

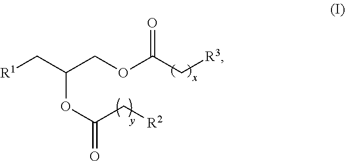

or a salt, co-crystal, tautomer, stereoisomer, solvate, hydrate, polymorph, or an isotopically enriched derivative thereof, wherein:
x is 1,2,3,4,5,6,7,8,9,10,11,12,13,14,15,16,17,18, 19,20,21,22,23, or 24;
y is 1,2,3,4,5,6,7,8,9,10,11,12,13,14,15,16,17,18, 19,20,21,22,23, or 24;
R¹ is a phosphoglycerol, phosphocholine, phosphoethanolamine, phosphoinositol, or phosphoserine moiety;
R² is halogen, optionally substituted acyl, optionally substituted branched or unbranched alkyl, optionally substituted carbocyclyl, optionally substituted heterocyclyl, optionally substituted aryl, —OR^{D1}, —N(R^{D1a})₂, —SR^{D1}, —SO₂, —NO₂, —N₃, or —CN;
R³ is halogen, optionally substituted acyl, optionally substituted branched or unbranched alkyl, optionally substituted carbocyclyl, optionally substituted heterocyclyl, optionally substituted aryl, —OR^{D1}, —N(R^{D1a})₂, —SR^{D1}, —SO₂, —NO₂, —N₃, or —CN;
wherein R^{D1} is independently hydrogen, optionally substituted acyl, optionally substituted alkyl, optionally substituted alkenyl, optionally substituted cycloalkynyl group, optionally substituted carbocyclyl, optionally substituted heterocyclyl, optionally substituted aryl, optionally substituted heteroaryl, or an oxygen protecting group when attached to an oxygen atom, or a sulfur protecting group when attached to a sulfur atom; and
wherein each occurrence of R^{D1a} is independently hydrogen, optionally substituted acyl, optionally substituted alkyl, optionally substituted alkenyl, optionally substituted linear alkynyl, optionally substituted cycloalkynyl group, optionally substituted carbocyclyl, optionally substituted heterocyclyl, optionally substituted aryl, optionally substituted heteroaryl, or a nitrogen protecting group; or optionally two instances of R^{D1a} are taken together with their intervening atoms to form a substituted or unsubstituted heterocyclic or substituted or unsubstituted heteroaryl ring.

In certain embodiments of Formula (I):
x is 1,2,3,4,5,6,7,8,9,10,11,12,13,14,15,16,17,18, 19,20,21,22,23, or 24;
y is 1, 2, 3, 4, 5, 6, 7, 8, 9, 10, 11, 12, 13, 14, 15, 16, 17, 18, 19, 20, 21, 22, 23, or 24;
R¹ is a phosphoglycerol, phosphocholine, phosphoethanolamine, phosphoinositol, or phosphoserine moiety;
R² is halogen, optionally substituted acyl, optionally substituted branched alkyl, optionally substituted carbocyclyl, optionally substituted heterocyclyl, optionally substituted aryl, —OR^{D1}, —N(R^{D1a})₂, —SR^{D1}, —SO₂, —NO₂, —N₃, or —CN;
R³ is halogen, optionally substituted acyl, optionally substituted branched alkyl, optionally substituted carbocyclyl, optionally substituted heterocyclyl, optionally substituted aryl, —OR^{D1}, —N(R^{D1a})₂, —SR^{D1}, SO₂, —NO₂, —N₃, or —CN;
wherein R^{D1} is independently hydrogen, optionally substituted acyl, optionally substituted alkyl, optionally substituted alkenyl, optionally substituted cycloalkynyl group, optionally substituted carbocyclyl, optionally substituted heterocyclyl, optionally substituted aryl, optionally substituted heteroaryl, or an oxygen protecting group when attached to an oxygen atom, or a sulfur protecting group when attached to a sulfur atom; and
wherein each occurrence of R^{D1a} is independently hydrogen, optionally substituted acyl, optionally substituted alkyl, optionally substituted alkenyl, optionally substituted linear alkynyl, optionally substituted cycloalkynyl group, optionally substituted carbocyclyl, optionally substituted heterocyclyl, optionally substituted aryl, optionally substituted heteroaryl, or a nitrogen protecting group; or optionally two instances of R^{D1a} are taken together with their intervening atoms to form a substituted or unsubstituted heterocyclic or substituted or unsubstituted heteroaryl ring.

In certain embodiments, the compound is of Formula (I):

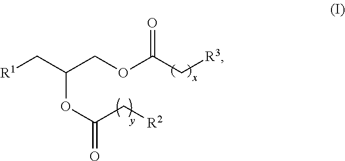

or a salt thereof.

In certain embodiments, the compound of Formula (I) is of Formula (I-A):

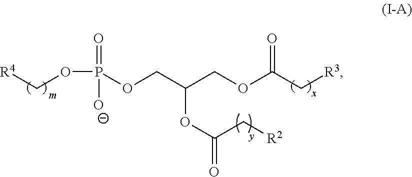

or a salt, solvate, hydrate, stereoisomer, polymorph, tautomer, isotopically enriched form, or prodrug thereof, wherein:
R⁴ is

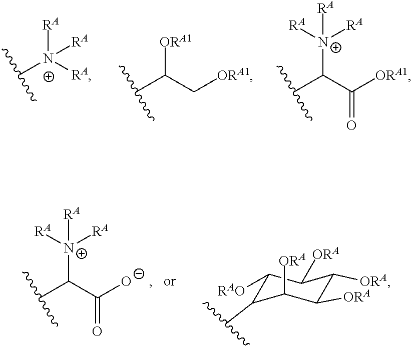

as valency permits;
each occurrence of R^{A} is independently hydrogen or unsubstituted alkyl;
each occurrence of R^{A1} is independently hydrogen, unsubstituted alkyl, or an oxygen protecting group; and
m is 1, 2, 3, 4, 5, or 6.

In certain embodiments, the compound of Formula (I) is of Formula (I-A):

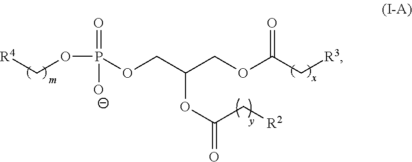

or a salt thereof.

In certain embodiments, the compound of Formula (I) is of Formula (I-A-1):

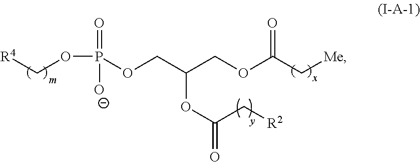

or a salt, solvate, hydrate, stereoisomer, polymorph, tautomer, isotopically enriched form, or prodrug thereof, wherein:
R⁴ is

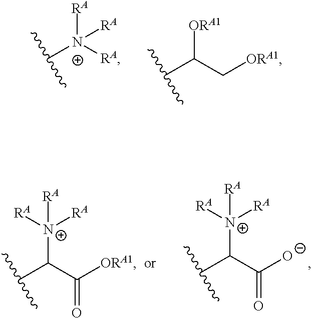

as valency permits;
each occurrence of R^{A} is independently hydrogen or unsubstituted alkyl;
each occurrence of R^{A1} is independently hydrogen, unsubstituted alkyl, or an oxygen protecting group; and
m is 1, 2, 3, 4, 5, or 6.

In certain embodiments, the compound of Formula (I) is of Formula (I-A-1):

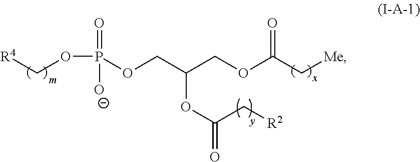

or a salt thereof.

In certain embodiments, the compound described herein, e.g., the compound of Formula (I) and/or (I-A), includes substituent R¹. In certain embodiments, R¹ is a phosphoglycerol, phosphocholine, phosphoethanolamine, phosphoinositol, or phosphoserine moiety. In certain embodiments, a phosphoglycerol moiety refers to a compound comprising phosphoglycerol. In certain embodiments, a phosphocholine moiety refers to a compound comprising phosphocholine. In certain embodiments, a phosphoethanolamine moiety refers to a compound comprising phosphoethanolamine. In certain embodiments, a phosphoinositol moiety refers to a compound comprising phosphoinositol. In certain embodiments, a phosphoserine moiety refers to a compound comprising phosphoserine. In certain embodiments, R¹ is an unsubstituted phosphoglycerol, unsubstituted phosphocholine, unsubstituted phosphoethanolamine, unsubstituted phosphoinositol, or unsubstituted phosphoserine moiety. In certain embodiments, R¹ is of the formula:

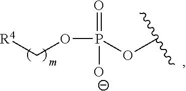

wherein m and R⁴ are as defined herein. In certain embodiments, m is 1, 2, 3, 4, 5, or 6. In certain embodiments, m is 1. In certain embodiments, m is 2. In certain embodiments, m is 3. In certain embodiments, m is 4. In certain embodiments, m is 5. In certain embodiments m is 6. In certain embodiments, R¹ is of the formula:

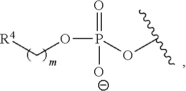

wherein m is 2, 3, or 4; R⁴ is

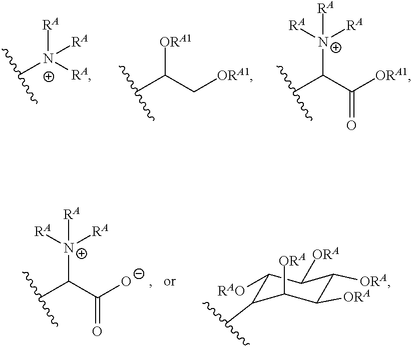

as valency permits, and R^{A} and R^{A1} are as defined herein. In certain embodiments, the compound of Formula (I) is of Formula (I-A-1), and m is 2. In certain embodiments, R⁴ is

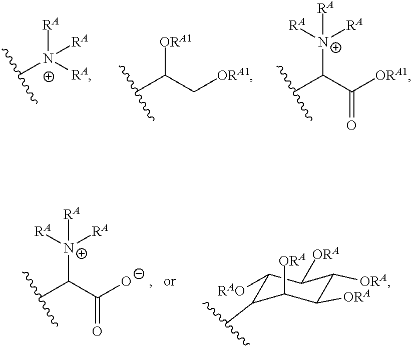

as valency permits, wherein R^{A} and R^{A1} are as defined herein. In certain embodiments, R⁴ is

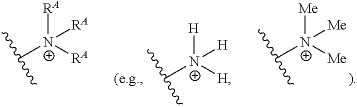

In certain embodiments, R⁴ is

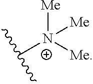 [SMILES: *[N+](C)(C)C]

In certain embodiments, R⁴ is

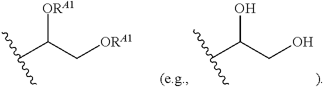

In certain embodiments, R⁴ is

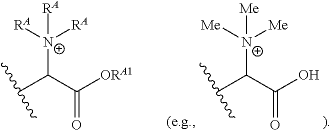

In certain embodiments, R⁴ is

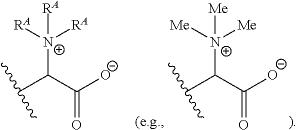

In certain embodiments, R⁴ is

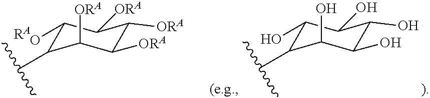

In certain embodiments, R⁴ is

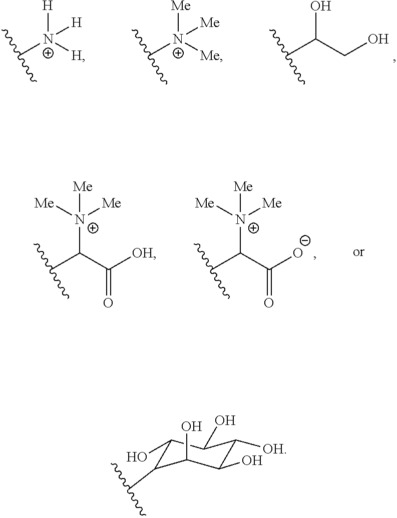

### R⁴ is

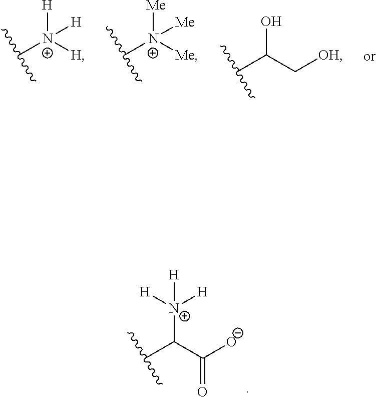

In certain embodiments, each occurrence of R^{A} is independently hydrogen or unsubstituted alkyl. In certain embodiments, at least one instance of R^{A} is hydrogen. In certain embodiments, at least one instance of R^{A} is unsubstituted alkyl (e.g., Me). In certain embodiments, each occurrence of R^{A1} is independently hydrogen or unsubstituted alkyl. In certain embodiments, at least one instance of R^{A1} is hydrogen. In certain embodiments, at least one instance of R^{A1} is unsubstituted alkyl (e.g., Me).

The compound of Formula (I) includes substituent x. In certain embodiments, x is 1, 2,3,4,5,6,7,8,9, 10, 11, 12, 13, 14, 15, 16, 17, 18, 19, 20, 21, 22, 23, 24, 25, 26, 27, or 28. In certain embodiments, x is 1, 2, 3, 4, 5, 6, 7, 8, 9, 10, 11, 12, 13, 14, 15, 16, 17, 18, 19, 20, 21, 22, 23, or 24. In certain embodiments, x is an integer between 1-2, 2-3, 3-4, 4-5, 5-7, 7-8, 8-10, 10-12, 12-14, 14-16, 16-18, 18-20, 20-24, inclusive. In certain embodiments, x is an integer between 12-14, 14-16, 16-17, 17-18, 18-19, 19-20, inclusive. In certain embodiments, x is 1. In certain embodiments, x is 2. In certain embodiments, x is 3. In certain embodiments, x is 4. In certain embodiments, x is 5. In certain embodiments x is 6. In certain embodiments, x is 7. In certain embodiments, x is 8. In certain embodiments, x is 9. In certain embodiments, x is 10. In certain embodiments, x is 11. In certain embodiments x is 12. In certain embodiments x is 12. In certain embodiments x is 13. In certain embodiments, x is 14. In certain embodiments, x is 15. In certain embodiments, x is 16. In certain embodiments, x is 17. In certain embodiments, x is 18. In certain embodiments x is 19. In certain embodiments x is 20. In certain embodiments x is 21. In certain embodiments x is 22. In certain embodiments x is 23. In certain embodiments x is 24. In certain embodiments x is 25. In certain embodiments x is 26.

The compound of Formula (I) includes substituent y. In certain embodiments, y is 1, 2,3,4,5,6,7,8,9, 10, 11, 12, 13, 14, 15, 16, 17, 18, 19, 20, 21, 22, 23, 24, 25, 26, 27, or 28. In certain embodiments, y is 1, 2, 3, 4, 5, 6, 7, 8, 9, 10, 11, 12, 13, 14, 15, 16, 17, 18, 19, 20, 21, 22, 23, or 24. In certain embodiments, y is an integer between 1-2, 2-3, 3-4, 4-5, 5-7, 7-8, 8-10, 10-12, 12-14, 14-16, 16-18, 18-20, 20-24, inclusive. In certain embodiments, y is an integer between 12-14, 14-16, 16-17, 17-18, 18-19, 19-20, inclusive. In certain embodiments, y is 1. In certain embodiments, y is 2. In certain embodiments, y is 3. In certain embodiments, y is 4. In certain embodiments, y is 5. In certain embodiments y is 6. In certain embodiments, y is 7. In certain embodiments, y is 8. In certain embodiments, y is 9. In certain embodiments, y is 10. In certain embodiments, y is 11. In certain embodiments y is 12. In certain embodiments y is 12. In certain embodiments y is 13. In certain embodiments, y is 14. In certain embodiments, y is 15. In certain embodiments, y is 16. In certain embodiments, y is 17. In certain embodiments, y is 18. In certain embodiments y is 19. In certain embodiments y is 20. In certain embodiments y is 21. In certain embodiments y is 22. In certain embodiments y is 23. In certain embodiments y is 24. In certain embodiments y is 25. In certain embodiments y is 26. In certain embodiments, x and y are the same. In certain embodiments, x and y are different. In certain embodiments, x and y are both 12, 13, 14, 15, or 16. In certain embodiments, x and y are both an integer between 12-14, 14-16, or 16-18, inclusive. In certain embodiments, x is 12, 13, 14, 15, or 16. In certain embodiments, y is 12, 13, 14, 15, or 16. In certain embodiments, x is an integer between 12-14, 14-16, or 16-18, inclusive. In certain embodiments, y is an integer between 12-14, 14-16, or 16-18, inclusive. In certain embodiments, x is 14. In certain embodiments, x is 15. In certain embodiments, y is 15. In certain embodiments, x is 14, and y is 15.

In certain embodiments, the compound described herein, e.g., the compound of Formula (I) and/or (I-A), includes substituents R² and R³. In certain embodiments, R² and R³ are the same. In certain embodiments, each of R² and R³ is halogen. In certain embodiments, each of R² and R³ is —OR^{D1}, and R^{D1} is optionally substituted acyl, optionally substituted aryl, optionally substituted heteroaryl, optionally substituted cycloalkynyl group, or optionally substituted linear alkynyl. In certain embodiments, each of R² and R³ is —Br or —N₃. In certain embodiments, each of R² and R³ is —OPh. In certain embodiments, each of R² and R³ is —N₃. In certain embodiments, each of R² and R³ is of the formula:

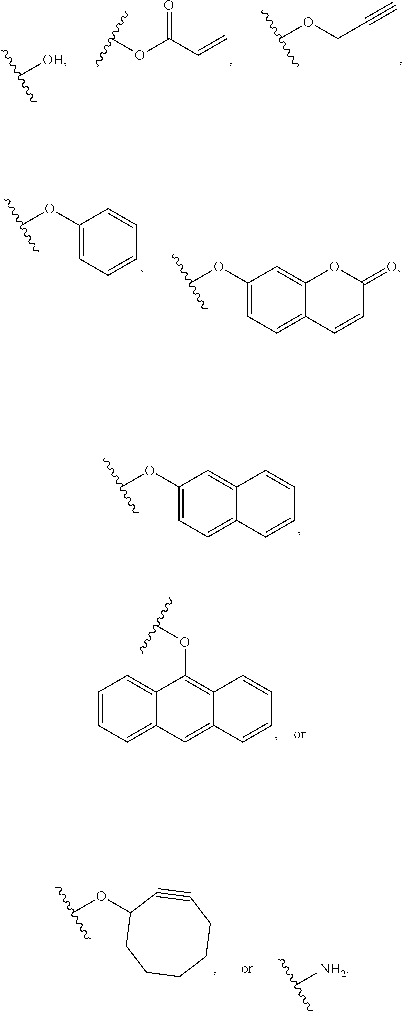

In certain embodiments, R² and R³ are different. In certain embodiments, R³ is linear alkyl (e.g., methyl) and R² is —OR^{D1}, and R^{D1} is optionally substituted acyl, optionally substituted aryl, optionally substituted heteroaryl, optionally substituted cycloalkynyl group, or optionally substituted linear alkynyl. In certain embodiments, R³ is linear C₁₋₆ alkyl (e.g., methyl) and R² is —OR^{D1}, and R^{D1} is optionally substituted acyl, optionally substituted aryl, optionally substituted heteroaryl, optionally substituted cycloalkynyl group, or optionally substituted linear alkynyl.

In certain embodiments, R² is optionally substituted acyl, halogen, optionally substituted linear alkyl, optionally substituted carbocyclyl, optionally substituted branched alkyl, optionally substituted heteroaryl, optionally substituted aryl, —OR^{D}, —N(R^{D1a})₂, —SR^{D1}, —SO₂, —NO₂, —N₃, or —CN. In certain embodiments, R² is not optionally substituted linear alkyl or optionally substituted branched alkyl. In certain embodiments, R² and/or R³ are each optionally substituted linear alkyl, only when the composition further comprises (e.g., physically encapsulates) an agent, for example, a therapeutic agent (e.g., small molecule therapeutic agent, a protein therapeutic agent, a nucleic acid therapeutic agent; diagnostic agent (e.g., fluorophore, for example, organic dyes (e.g., fluorescein, rhodamine, AMCA), or biological fluorophores (e.g., green fluorescent protein, phycoerythrin, allophycocyanin; Sulforhodamine B, indocyanine green, methylene blue, or coumarin). In certain embodiments, R² and R³ are both optionally substituted linear alkyl, only when the composition further comprises (e.g., physically encapsulates) an agent, for example, a therapeutic agent (e.g., small molecule therapeutic agent, a protein therapeutic agent, a nucleic acid therapeutic agent; diagnostic agent (e.g., fluorophore, for example, organic dyes (e.g., fluorescein, rhodamine, AMCA), or biological fluorophores (e.g., green fluorescent protein, phycoerythrin, allophycocyanin; Sulforhodamine B, indocyanine green, methylene blue, or coumarin).

In certain embodiments, R² is halogen, optionally substituted acyl, optionally substituted branched alkyl, optionally substituted carbocyclyl, optionally substituted heterocyclyl, optionally substituted aryl, —OR^{D1}, —N(R^{D1a})₂, —SR^{D1}, SO₂, —NO₂, —N₃, or —CN. In certain embodiments, R² is halogen (e.g., F, Cl, Br, or I). In certain embodiments, R² is Br. In certain embodiments, R² is optionally substituted acyl (e.g., —C(═O)Me). In certain embodiments, R² is optionally substituted branched alkyl (e.g., substituted or unsubstituted branched C₃₋₆ alkyl, for example, isopropyl, t-butyl, sec-butyl, iso-butyl). In certain embodiments, R² is optionally substituted carbocyclyl (e.g., substituted or unsubstituted, 3- to 10-membered, monocyclic carbocyclyl comprising zero, one, or two double bonds in the carbocyclic ring system). In certain embodiments, R² is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, R² is optionally substituted aryl (e.g., substituted or unsubstituted, 6- to 10-membered aryl). In certain embodiments, R² is benzyl. In certain embodiments, R² is substituted or unsubstituted phenyl. In certain embodiments, R² is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, R² is —OR^{D1} (e.g., —OH or —OMe). In certain embodiments, R² is —OH. In certain embodiments, R² is —OR^{D1}, and R^{D1} is optionally substituted acyl, optionally substituted aryl, optionally substituted heteroaryl, optionally substituted cycloalkynyl group, or optionally substituted linear alkynyl. In certain embodiments, R² is —OR^{D1}, and R^{D1} is

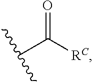

optionally substituted linear alkynyl, optionally substituted phenyl, optionally substituted naphthyl, optionally substituted anthryl, optionally substituted 2H-chromen-2-one (coumarin), optionally substituted cyclooctynyl, optionally substituted dibenzocyclooctyne, or optionally substituted aza-dibenzocyclooctyne; and R^{C} is hydrogen, optionally substituted alkyl, or optionally substituted alkenyl. In certain embodiments, R^{C} is hydrogen. In certain embodiments, R^{C} is optionally substituted alkyl (e.g., substituted or unsubstituted C₁₋₆ alkyl). In certain embodiments, R^{C} is optionally substituted alkenyl (e.g., substituted or unsubstituted C₂₋₆ alkenyl). In certain embodiments, R² is —OR^{D1}, and R^{D1} is a bulky (e.g., molecular weight between 90-300 g/mol), and/or hydrophobic moiety (e.g., optionally substituted C₁₋₁₂ alkyl, optionally substituted C₁₂₋₂₄ alkyl, optionally substituted C₂₋₁₂ alkenyl, optionally substituted C₁₂₋₂₄ alkenyl, optionally substituted linear or cyclic C₂₋₁₂ alkynyl, optionally substituted linear or cyclic C₁₂₋₂₄ alkynyl, C₃₋₁₄ carbocyclyl). In certain embodiments, R² is of the formula:

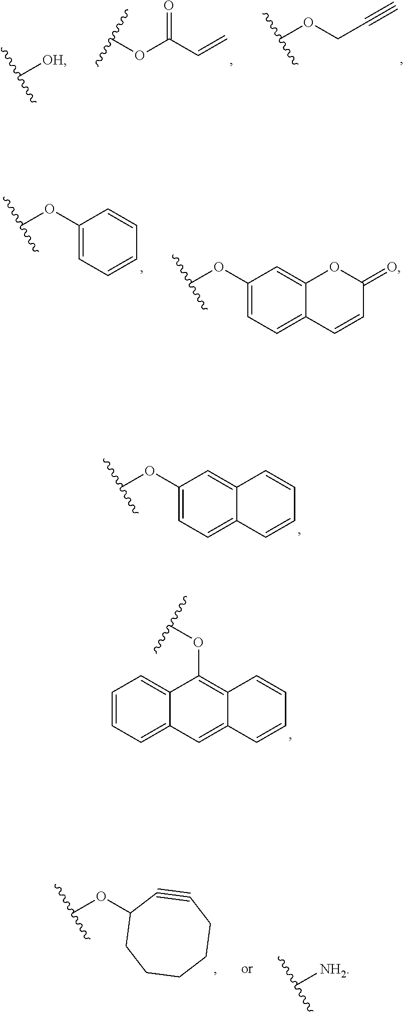

In certain embodiments, R² is of the formula:

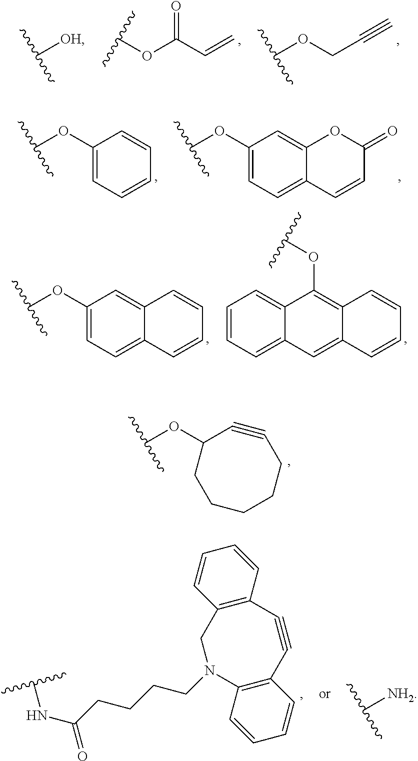

In certain embodiments, R² is

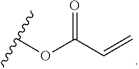 [SMILES: *OC(=O)C=C]

In certain embodiments, R² is

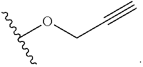 [SMILES: *OCC#C]

In certain embodiments, R² is

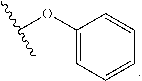 [SMILES: *Oc1ccccc1]

In certain embodiments, R² is

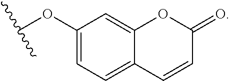 [SMILES: *Oc1ccc2ccc(=O)oc2c1]

In certain embodiments, R² is

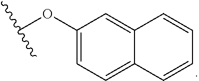 [SMILES: *Oc1ccc2ccccc2c1]

In certain embodiments, R² is

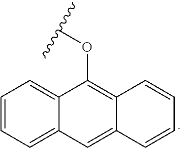 [SMILES: *Oc1c2ccccc2cc2ccccc12]

In certain embodiments, R² is

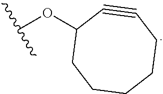 [SMILES: *OC1C#CCCCCC1]

In certain embodiments, R² is

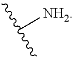 [SMILES: *N]

In certain embodiments, R² is —N(R^{D1a})₂ (e.g., —NMe₂). In certain embodiments, R² is of the formula:

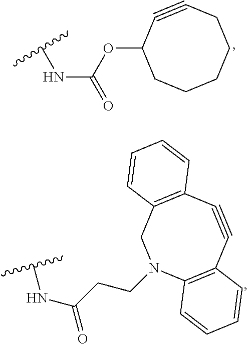 [SMILES: *NC(=O)OC1C#CCCCCC1 ; *NC(=O)CCN1Cc2ccccc2C#Cc2ccccc21]

or —SH. In certain embodiments, R² is of the formula:

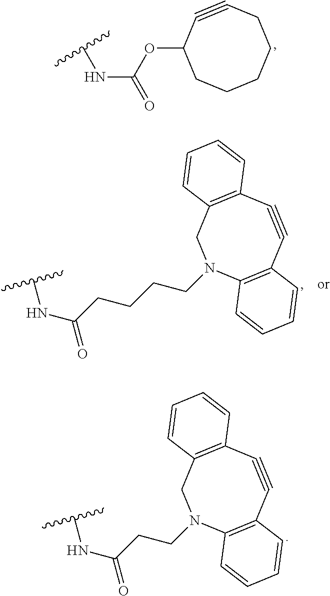 [SMILES: *NC(=O)OC1C#CCCCCC1 ; *NC(=O)CCCCN1Cc2ccccc2C#Cc2ccccc21 ; *NC(=O)CCN1Cc2ccccc2C#Cc2ccccc21]

In certain embodiments, R² is of the formula:

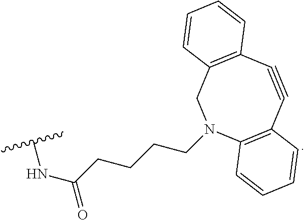 [SMILES: *NC(=O)CCCCN1Cc2ccccc2C#Cc2ccccc21]

In certain embodiments, R² is of the formula:

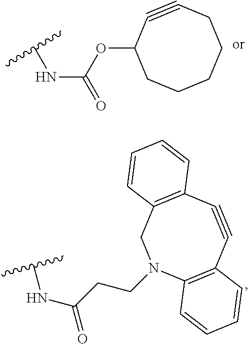 [SMILES: *NC(=O)OC1C#CCCCCC1 ; *NC(=O)CCN1Cc2ccccc2C#Cc2ccccc21]

In certain embodiments, R² is —NH₂. In certain embodiments, R² is —SR^{D1} (e.g., —SMe). In certain embodiments, R² is —SH. In certain embodiments, R² is —SO₂. In certain embodiments, R² is —NO₂. In certain embodiments, R² is —N₃. In certain embodiments, R² is —CN. In certain embodiments, R² is —Br or —N₃. In certain embodiments, R² is of the formula:

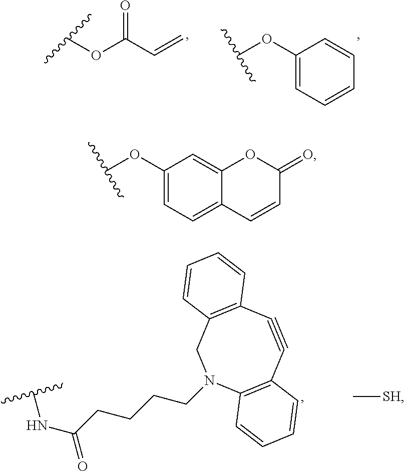

or —N₃. In certain embodiments, R² is

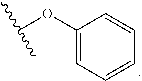 [SMILES: *Oc1ccccc1]

In certain embodiments, at least one instance of R² or R³ is —OR^{D1}, —N(R^{D1a})₂ or —SR^{D1}, and R^{D1} and R^{D1a} are as defined herein. In certain embodiments, at least one instance of R² or R³ is —N(R^{D1a})₂, —N(R^{D1a})C(═O)OR^{D1}, or —SR^{D1}; and R^{D1} is hydrogen, optionally substituted alkyl, optionally substituted aryl, optionally substituted heteroaryl, or optionally substituted cycloalkynyl group; and each instance of R^{D1a} is independently hydrogen, optionally substituted acyl, optionally substituted alkyl, or optionally substituted alkenyl. In certain embodiments, R² is —N(R^{D1a})₂, —N(R^{D1a})C(═O)OR^{D1}, or —SR^{D1}; and R^{D1} is hydrogen, optionally substituted alkyl, optionally substituted aryl, optionally substituted heteroaryl, or optionally substituted cycloalkynyl group; and each instance of R^{D1a} is independently hydrogen, optionally substituted acyl, optionally substituted alkyl, or optionally substituted alkenyl. In certain embodiments, R^{D1} is hydrogen. In certain embodiments, R^{D1} is optionally substituted acyl (e.g., —C(═O)Me). In certain embodiments, R^{D1} is

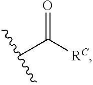

and R^{C} is hydrogen, optionally substituted alkyl, or optionally substituted alkenyl. In certain embodiments, R^{D1} is

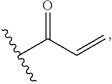 [SMILES: *C(=O)C=C]

In certain embodiments, R^{C} is hydrogen. In certain embodiments, R^{C} is optionally substituted alkyl (e.g., substituted or unsubstituted C₁₋₆ alkyl). In certain embodiments, R^{C} is optionally substituted alkenyl (e.g., substituted or unsubstituted C₂₋₆ alkenyl).

In certain embodiments, R^{D1} is optionally substituted alkyl (e.g., substituted or unsubstituted C₁₋₆ alkyl). In certain embodiments, R^{D1} is substituted or unsubstituted methyl. In certain embodiments, R^{D1} is substituted or unsubstituted ethyl. In certain embodiments, R^{D1} is substituted or unsubstituted propyl. In certain embodiments, R^{D1} is optionally substituted alkenyl (e.g., substituted or unsubstituted C₂₋₆ alkenyl). In certain embodiments, R^{D1} is optionally substituted alkynyl (e.g., substituted or unsubstituted C₂₋₆ alkynyl). In certain embodiments, R^{D1} is optionally substituted linear alkynyl. In certain embodiments, R^{D1} is

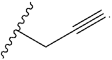 [SMILES: *CC#C]

In certain embodiments, R^{D1} is optionally substituted cyclic alkynyl or optionally substituted cycloalkynyl (e.g., a carbocyclyl group with at least one alkynyl bond). In certain embodiments, R^{D1} is optionally substituted cyclooctyne. In certain embodiments, R^{D1} is optionally substituted 2H-chromen-2-one (coumarin), optionally substituted cyclooctynyl, optionally substituted dibenzocyclooctyne, or optionally substituted aza-dibenzocyclooctyne. In certain embodiments, R^{D1} is

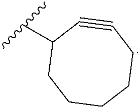 [SMILES: *C1C#CCCCCC1]

In certain embodiments, R^{D1} is optionally substituted carbocyclyl (e.g., substituted or unsubstituted, 3- to 7-membered, monocyclic carbocyclyl comprising zero, one, or two double bonds in the carbocyclic ring system). In certain embodiments, R^{D1} is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, R^{D1} is optionally substituted aryl (e.g., substituted or unsubstituted, 6- to 10-membered aryl). In certain embodiments, R^{D1} is benzyl. In certain embodiments, R^{D1} is substituted or unsubstituted phenyl. In certain embodiments, R^{D1} is phenyl. In certain embodiments, R^{D1} is optionally substituted phenyl, optionally substituted naphthyl, optionally substituted anthryl. In certain embodiments, R^{D1} is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, R^{D1} is an oxygen protecting group when attached to an oxygen atom (e.g., silyl, TBDPS, TBDMS, TIPS, TES, TMS, MOM, THP, t-Bu, Bn, allyl, acetyl, pivaloyl, benzoyl). In certain embodiments, R^{D1} is a sulfur protecting group when attached to a sulfur atom.

In certain embodiments, at least one instance of R^{D1a} is hydrogen. In certain embodiments, at least one instance of R^{D1a} is optionally substituted acyl (e.g., —C(═O)Me). In certain embodiments, at least one R^{D1a} is optionally substituted alkyl (e.g., substituted or unsubstituted C₁₋₆ alkyl). In certain embodiments, at least one instance of R^{D1a} is substituted or unsubstituted methyl. In certain embodiments, at least one instance of R^{D1a} is substituted or unsubstituted ethyl. In certain embodiments, at least one instance of R^{D1a} is substituted or unsubstituted propyl. In certain embodiments, at least one instance of R^{D1a} is optionally substituted alkenyl (e.g., substituted or unsubstituted C₂₋₆ alkenyl). In certain embodiments, at least one instance of R^{D1a} is optionally substituted alkynyl (e.g., substituted or unsubstituted C₂₋₆ alkynyl). In certain embodiments, at least one instance of R^{D1a} is optionally substituted carbocyclyl (e.g., substituted or unsubstituted, 3- to 7-membered, monocyclic carbocyclyl comprising zero, one, or two double bonds in the carbocyclic ring system). In certain embodiments, at least one instance of R^{D1a} is optionally substituted cyclic alkynyl or optionally substituted cycloalkynyl (e.g., a carbocyclyl group with at least one alkynyl bond). In certain embodiments, at least one instance of R^{D1a} is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R^{D1a} is optionally substituted aryl (e.g., substituted or unsubstituted, 6- to 10-membered aryl). In certain embodiments, at least one instance of R^{D1a} is benzyl. In certain embodiments, at least one instance of R^{D1a} is substituted or unsubstituted phenyl. In certain embodiments, at least one instance of R^{D1a} is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, at least one instance of R^{D1a} is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)). In certain embodiments, two instances of R^{D1a} are taken together with their intervening atoms to form a optionally substituted heterocyclic ring (e.g., substituted or unsubstituted, 5- to 10-membered monocyclic or bicyclic heterocyclic ring, wherein one or two atoms in the heterocyclic ring are independently nitrogen, oxygen, or sulfur) or optionally substituted heteroaryl ring (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, one instance of R^{D1a} is

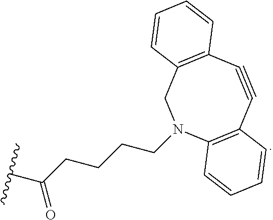 [SMILES: *C(=O)CCCCN1Cc2ccccc2C#Cc2ccccc21]

In certain embodiments, one instance of R^{D1a} is hydrogen and one instance of R^{D1a} is

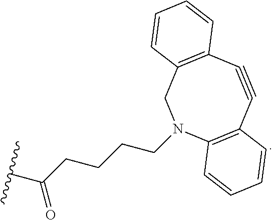 [SMILES: *C(=O)CCCCN1Cc2ccccc2C#Cc2ccccc21]

In certain embodiments, R³ is optionally substituted acyl, halogen, optionally substituted linear alkyl, optionally substituted carbocyclyl, optionally substituted branched alkyl, optionally substituted heteroaryl, optionally substituted aryl, —OR^{D}, —N(R^{D1a})₂, —SR^{D1}, —SO₂, —NO₂, —N₃, or —CN. In certain embodiments, R³ is halogen, optionally substituted acyl, optionally substituted branched alkyl, optionally substituted carbocyclyl, optionally substituted heterocyclyl, optionally substituted aryl, —OR^{D1}, —N(R^{D1a})₂—SR^{D1}, —SO₂, —NO₂, —N₃, or —CN. In certain embodiments, R³ is halogen, optionally substituted acyl, optionally substituted linear alkyl, optionally substituted carbocyclyl, optionally substituted heterocyclyl, optionally substituted aryl, —OR^{D1}, —N(R^{D1a})₂, —SR^{D1}, —SO₂, —NO₂, —N₃, or —CN. In certain embodiments, R³ is optionally substituted linear alkyl. In certain embodiments, R³ is optionally substituted C₁₋₄ linear alkyl. In certain embodiments, R³ is methyl. In certain embodiments, x is 15 and R³ is methyl. In certain embodiments, R³ is halogen (e.g., F, Cl, Br, or I). In certain embodiments, R³ is Br. In certain embodiments, R³ is optionally substituted acyl (e.g., —C(═O)Me). In certain embodiments, R³ is optionally substituted branched alkyl (e.g., substituted or unsubstituted branched C₃₋₆ alkyl, for example, isopropyl, t-butyl, sec-butyl, iso-butyl). In certain embodiments, R³ is optionally substituted carbocyclyl (e.g., substituted or unsubstituted, 3- to 10-membered, monocyclic carbocyclyl comprising zero, one, or two double bonds in the carbocyclic ring system). In certain embodiments, R³ is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, R³ is optionally substituted aryl (e.g., substituted or unsubstituted, 6- to 10-membered aryl). In certain embodiments, R³ is benzyl. In certain embodiments, R³ is substituted or unsubstituted phenyl. In certain embodiments, R³ is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, R³ is —OR^{D1} (e.g., —OH or —OMe). In certain embodiments, R³ is —OH. In certain embodiments, R² is —OR^{D1}, and R^{D1} is optionally substituted acyl, optionally substituted aryl, optionally substituted heteroaryl, optionally substituted cycloalkynyl group, or optionally substituted linear alkynyl. In certain embodiments, R³ is —OR^{D1}, and R^{D1} is

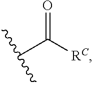

optionally substituted linear alkynyl, optionally substituted phenyl, optionally substituted naphthyl, optionally substituted anthryl, optionally substituted 2H-chromen-2-one (coumarin), optionally substituted cyclooctynyl, optionally substituted dibenzocyclooctyne, or optionally substituted aza-dibenzocyclooctyne; and R^{C} is hydrogen, optionally substituted alkyl, or optionally substituted alkenyl. In certain embodiments, R^{C} is hydrogen. In certain embodiments, R^{C} is optionally substituted alkyl (e.g., substituted or unsubstituted C₁₋₆ alkyl). In certain embodiments, R^{C} is optionally substituted alkenyl (e.g., substituted or unsubstituted C₂₋₆ alkenyl). In certain embodiments, R³ is of the formula:

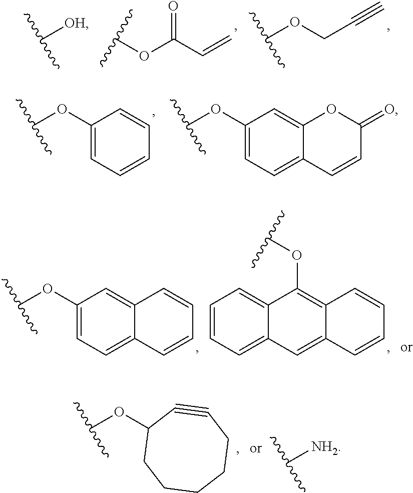

In certain embodiments, R³ is

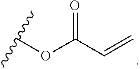 [SMILES: *OC(=O)C=C]

In certain embodiments, R³ is

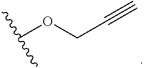 [SMILES: *OCC#C]

In certain embodiments, R is

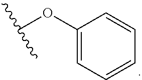 [SMILES: *Oc1ccccc1]

In certain embodiments, R³ is

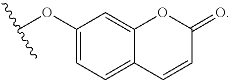 [SMILES: *Oc1ccc2ccc(=O)oc2c1]

In certain embodiments, R³ is

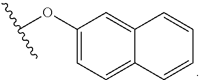 [SMILES: *Oc1ccc2ccccc2c1]

In certain embodiments, R³ is

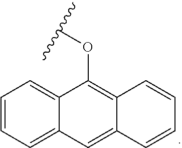 [SMILES: *Oc1c2ccccc2cc2ccccc12]

In certain embodiments, R³ is

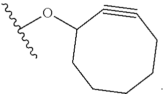 [SMILES: *OC1C#CCCCCC1]

In certain embodiments, R³ is

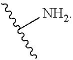 [SMILES: *N]

In certain embodiments, R³ is —N(R^{D1a})₂ (e.g., —NMe₂). In certain embodiments, R³ is of the formula:

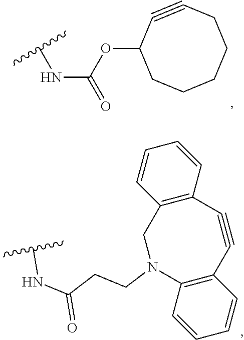 [SMILES: *NC(=O)OC1C#CCCCCC1 ; *NC(=O)CCN1Cc2ccccc2C#Cc2ccccc21]

or —SH. In certain embodiments, R³ is of the formula:

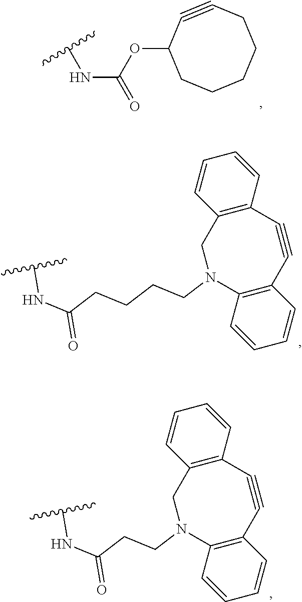 [SMILES: *NC(=O)OC1C#CCCCCC1 ; *NC(=O)CCCCN1Cc2ccccc2C#Cc2ccccc21 ; *NC(=O)CCN1Cc2ccccc2C#Cc2ccccc21]

or —SH. In certain embodiments, R³ is of the formula:

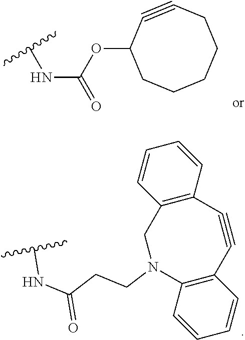 [SMILES: *NC(=O)OC1C#CCCCCC1 ; *NC(=O)CCN1Cc2ccccc2C#Cc2ccccc21]

In certain embodiments, R³ is of the formula:

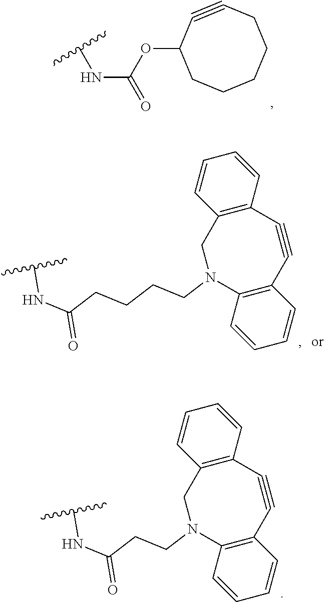 [SMILES: *NC(=O)OC1C#CCCCCC1 ; *NC(=O)CCCCN1Cc2ccccc2C#Cc2ccccc21 ; *NC(=O)CCN1Cc2ccccc2C#Cc2ccccc21]

In certain embodiments, R³ is of the formula:

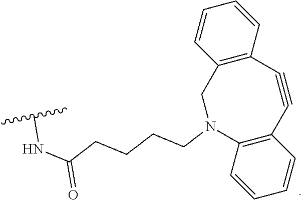 [SMILES: *NC(=O)CCCCN1Cc2ccccc2C#Cc2ccccc21]

In certain embodiments, R³ is —NH₂. In certain embodiments, R³ is —SR^{D1} (e.g., —SMe). In certain embodiments, R³ is —SO₂. In certain embodiments, R³ is —NO₂. In certain embodiments, R³ is —N₃. In certain embodiments, R³ is —CN. In certain embodiments, R³ is —Br or —N₃. In certain embodiments, R³ is methyl or —N₃. In certain embodiments, R³ is —N(R^{D1a})₂, —N(R^{D1a})C(═O)OR^{D1}, or —SR^{D1}; and R^{D1} is hydrogen, optionally substituted alkyl, optionally substituted aryl, optionally substituted heteroaryl, or optionally substituted cycloalkynyl group; and each instance of R^{D1a} is independently hydrogen, optionally substituted acyl, optionally substituted alkyl, or optionally substituted alkenyl.

In certain embodiments, the compound of Formula (I) is of formula:

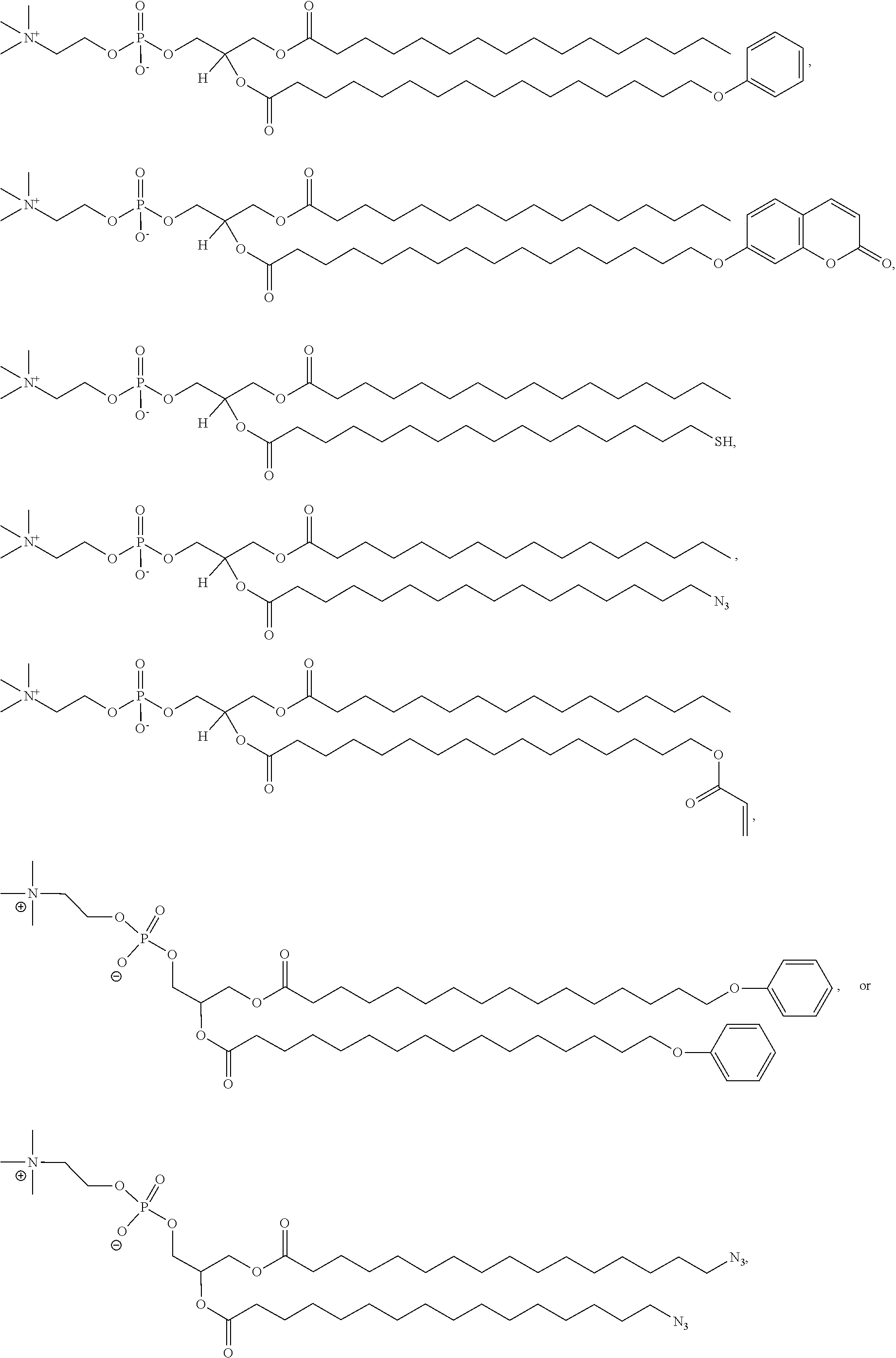

or a salt, co-crystal, tautomer, stereoisomer, solvate, hydrate, polymorph, or an isotopically enriched derivative thereof. In certain embodiments, the compound of Formula (I) is of formula:

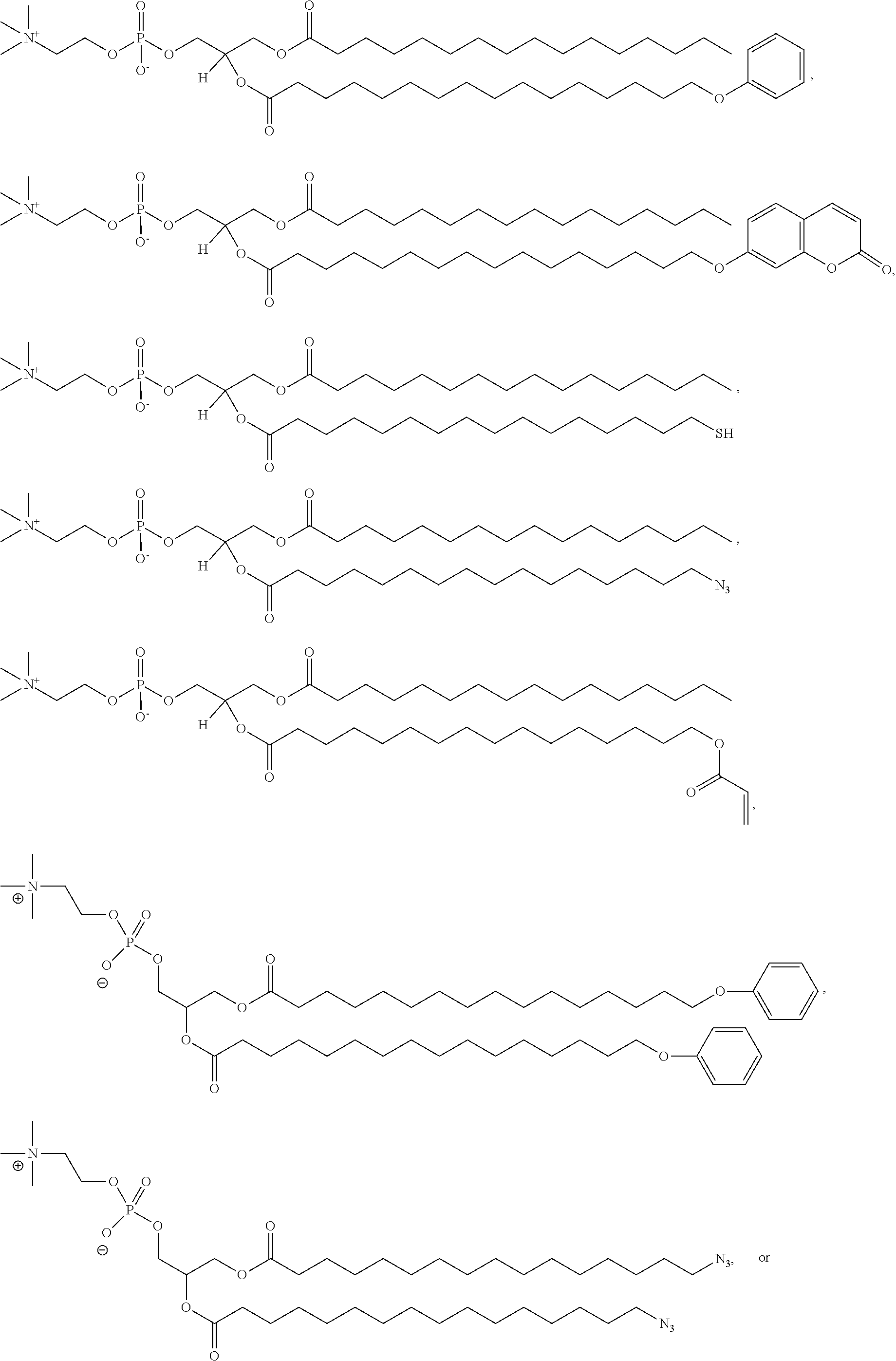

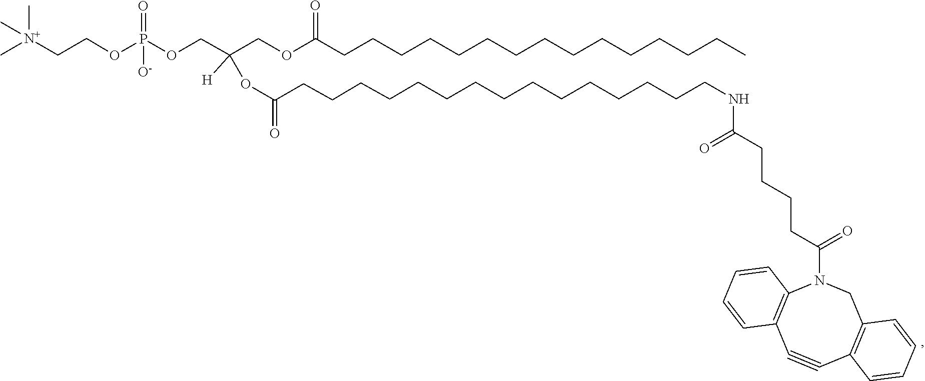 [SMILES: CCCCCCCCCCCCCCCC(=O)OCC(COP(=O)([O-])OCC[N+](C)(C)C)OC(=O)CCCCCCCCCCCCCCCNC(=O)CCCCC(=O)N1Cc2ccccc2C#Cc2ccccc21]

or a salt, co-crystal, tautomer, stereoisomer, solvate, hydrate, polymorph, or an isotopically enriched derivative thereof.

In certain embodiments, the compound of Formula (I) is of formula:

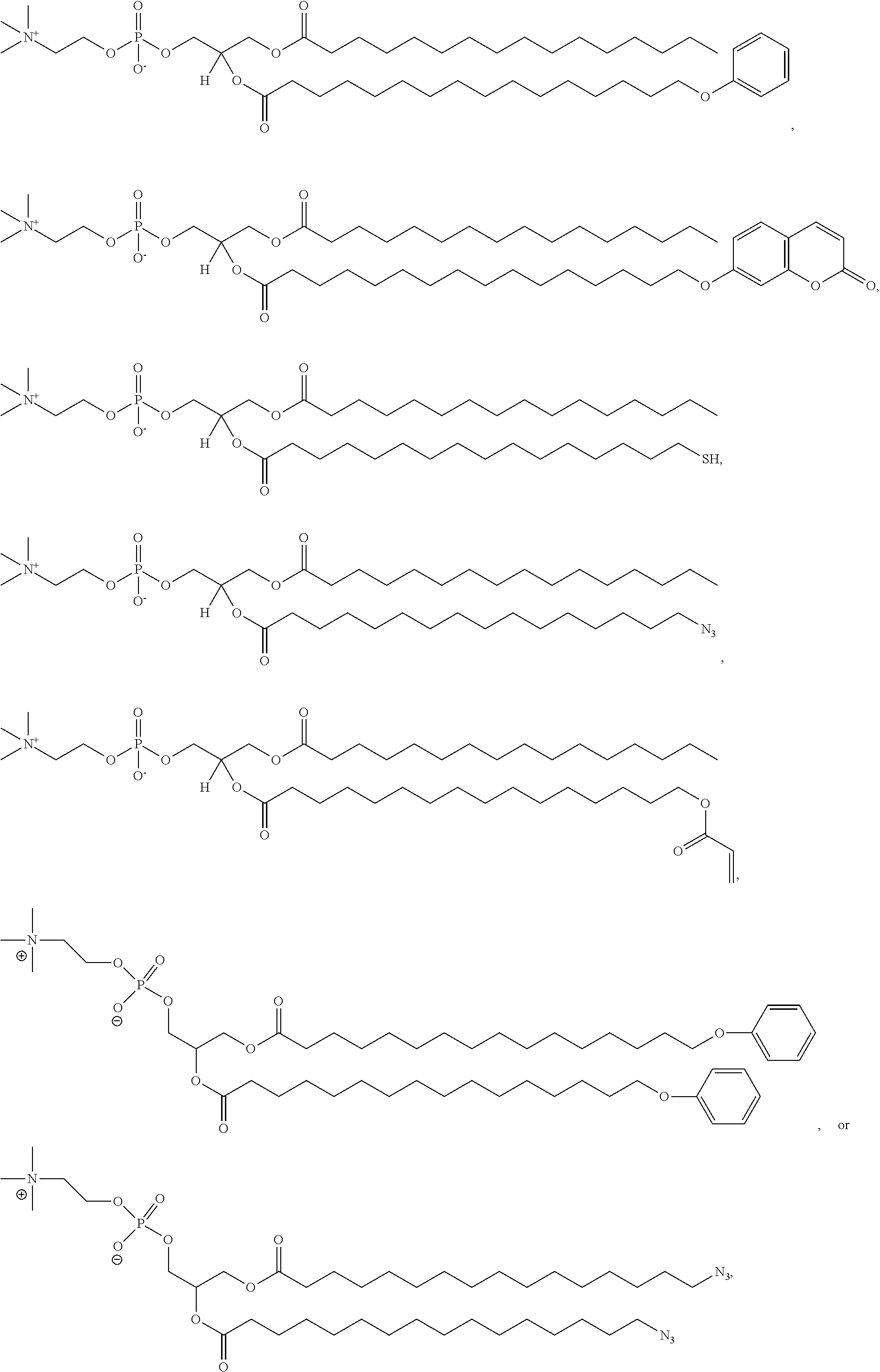

or a salt thereof. In certain embodiments, the compound of Formula (I) is of formula:

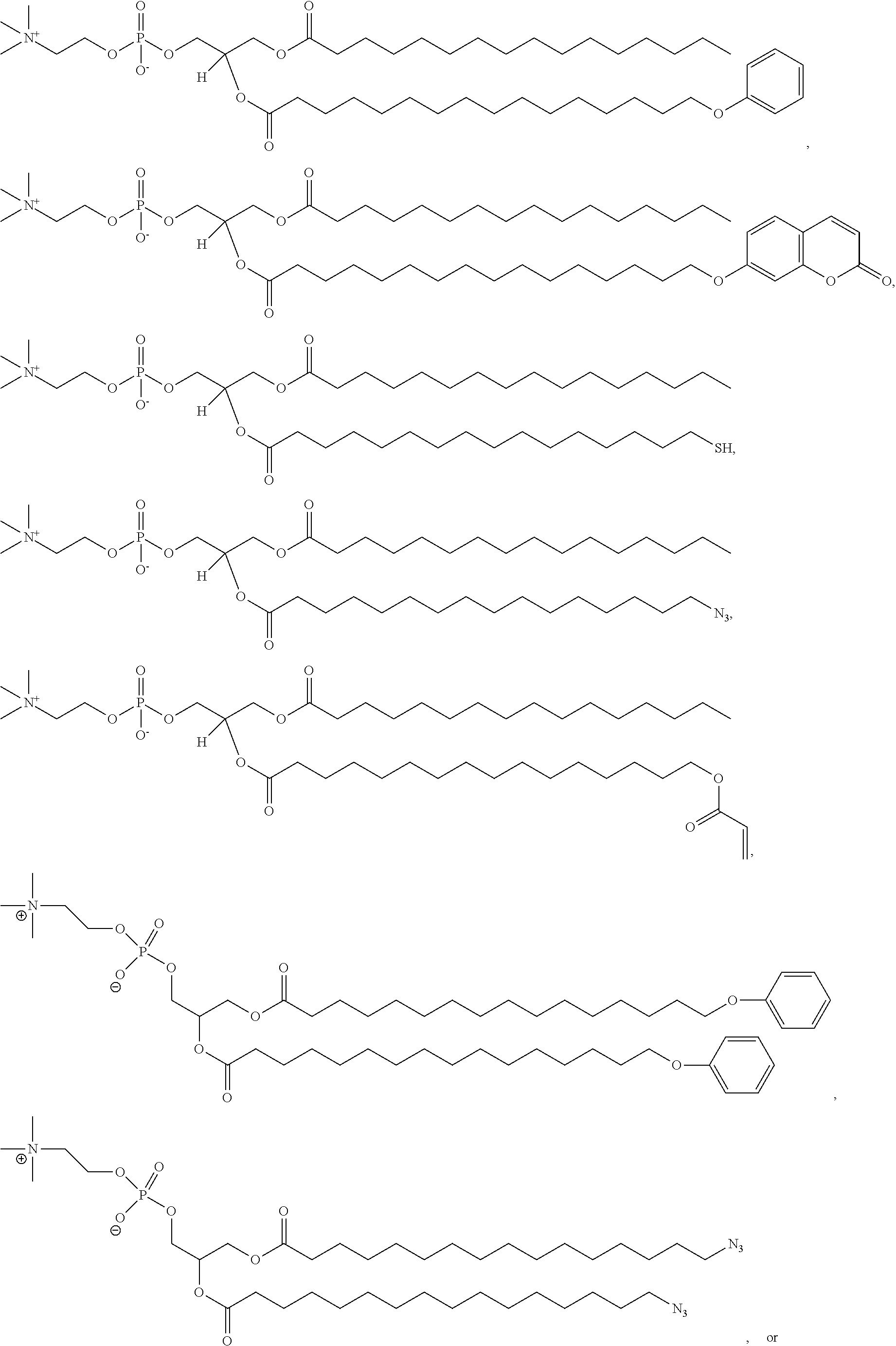

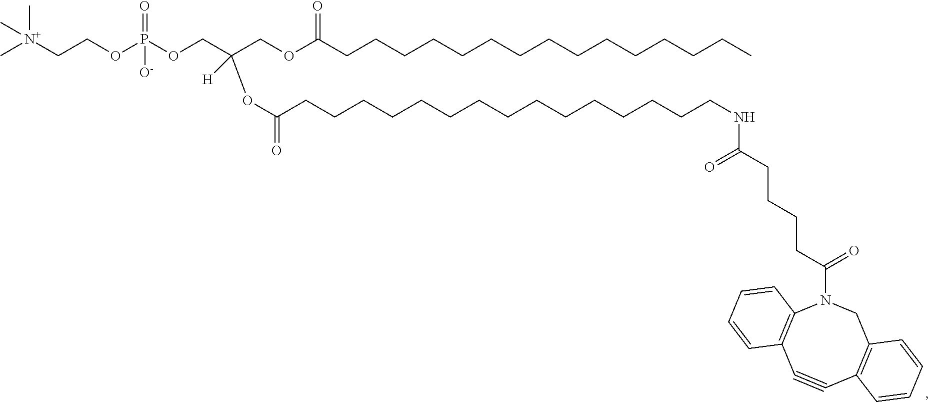 [SMILES: CCCCCCCCCCCCCCCC(=O)OCC(COP(=O)([O-])OCC[N+](C)(C)C)OC(=O)CCCCCCCCCCCCCCCNC(=O)CCCCC(=O)N1Cc2ccccc2C#Cc2ccccc21]

or a salt thereof.

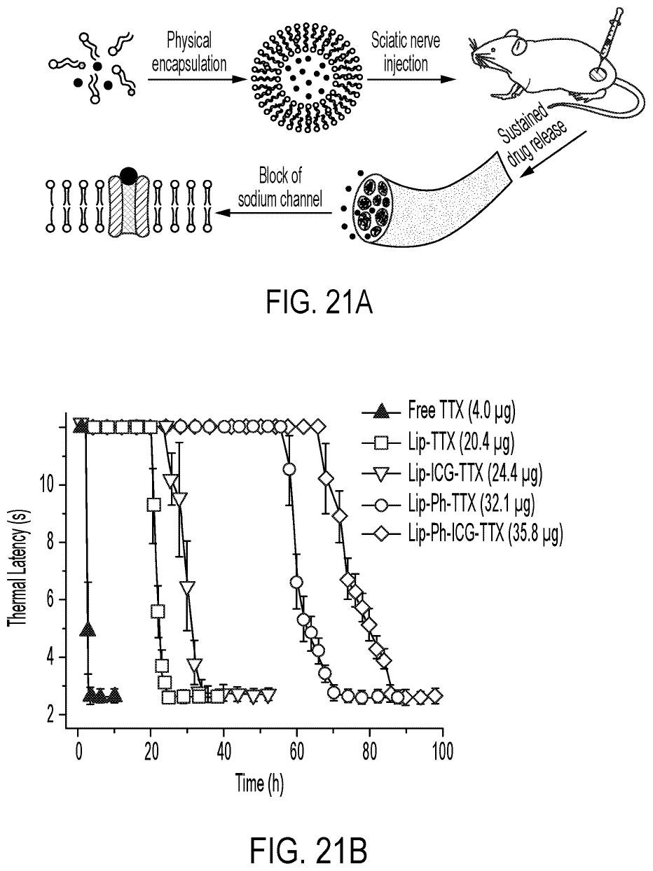

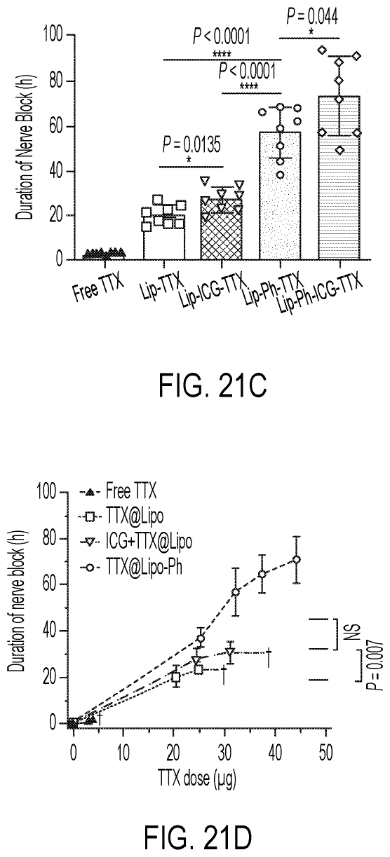

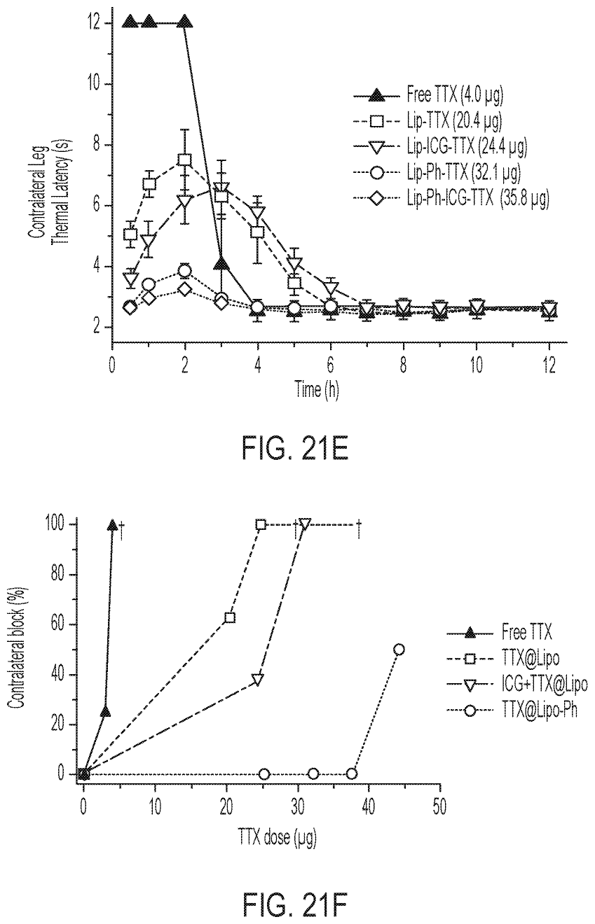

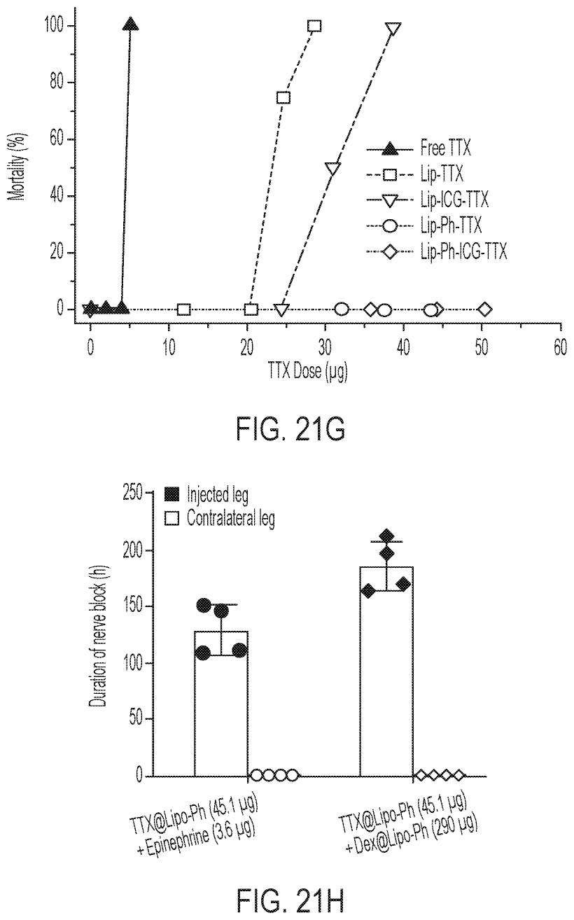

In another aspect, provided is a composition in the form of a particle (e.g., liposome), wherein a compound comprising an aromatic group (e.g., fluorophore, or dye, such as indocyanine green) is physically, non-covalently incorporated by the particle, which also comprises a local anesthetic agent (e.g., TTX). In certain embodiments, provided is a liposome comprising indocyanine green physically, non-covalently incorporated by the liposome, which also comprises TTX. In certain embodiments, provided is the liposome of Lip-ICG-TTX (as shown in FIG. 21 and described in Example 1).

In certain embodiments, the composition described herein further comprises one or more agents, for example, an agent (e.g., therapeutic agent, diagnostic agent). In certain embodiments, at least one of the one or more agents is a therapeutic agent or diagnostic agent. In certain embodiments, the therapeutic agent is an antibiotic agent, chemotherapeutic agent, anesthetic agent, anti-inflammatory agent, analgesic agent, anti-fibrotic agent, anti-sclerotic agent, or anticoagulant agent. Therapeutic agents, in certain embodiments, may include, but are not limited to, antimicrobial agents, antibiotics, anesthetics, anti-inflammatories, chemotherapeutic agents, analgesics, anti-fibrotics, anti-sclerotics, and anticoagulants. Therapeutic agents may include, but are not limited to, antibiotics, anesthetics, anti-inflammatories, analgesics, anti-fibrotics, anti-sclerotics, and anticoagulants. In certain embodiments, the therapeutic agent is an antimicrobial agent. In certain embodiments, the therapeutic agent is an antibiotic agent. In certain embodiments, the therapeutic agent is a chemotherapeutic agent. In certain embodiments, the therapeutic agent is an anesthetic agent. In certain embodiments, the therapeutic agent is an anti-inflammatory agent. In certain embodiments, the therapeutic agent is an analgesic agent. In certain embodiments, the therapeutic agent is an anti-fibrotic agent. In certain embodiments, the therapeutic agent is an anti-sclerotic agent. In certain embodiments, the therapeutic agent is an anticoagulant agent. In certain embodiments, the therapeutic agent is present as a pharmaceutically acceptable salt or a free base of the active agent. In certain embodiments, the therapeutic agent is present as a pharmaceutically acceptable salt of the active agent.

In certain embodiments, the therapeutic agent is an antimicrobial agent. In certain embodiments, the therapeutic agent is an antibiotic. Any antibiotic may be used in the system. In certain embodiments the antibiotic is approved for use in humans or other animals. In certain embodiments the antibiotic is approved for use by the U.S. Food & Drug Administration. In certain embodiments, the antibiotic may be selected from the group consisting of cephalosporins, quinolones, polypeptides, macrolides, penicillins, and sulfonamides. Exemplary antibiotics may include, but are not limited to, ciprofloxacin, cefuroxime, cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftobiprole, enoxacin, gatifloxacin, levofloxacin, lomefloxacin, moxifloxacin, norfloxacin, ofloxacin, trovafloxacin, bacitracin, colistin, polymyxin B, azithromycin, clarithromycin, dirithromycin, erythromycin, roxithromycin, troleandomycin, telithromycin, spectinomycin, amoxicillin, ampicillin, azlocillin, carbenicillin, cloxacillin, dicloxacillin, flucloxacillin, mezlocillin, meticillin, nafcillin, oxacillin, penicillin, piperacillin, ticarcillin, mafenide, sulfacetamide, sulfamethizole, sulfasalazine, sulfisoxazole, trimethoprim, and trimethoprim-sulfamethoxazole. In certain embodiments, the therapeutic agent is doxorubicin.

In certain embodiments, the therapeutic agent is an antibiotic agent, anesthetic agent, anti-inflammatory agent, analgesic agent, anti-fibrotic agent, anti-sclerotic agent, anticoagulant agent, or diagnostic agent. In certain embodiments, the antibiotic is a quinolone, for example, a fluoroquinolone. In certain embodiments, the antibiotic is a carbapenem. In certain embodiments, the antibiotic is a quinolone (e.g., fluoroquinolone) or a beta lactam antibiotic (e.g., penicillin, cephalosporin (e.g., ceftriaxone)). In certain embodiments, the antibiotic is amoxicillin, azithromicicn, cefuroxime, ceftriaxone, trimethoprim, levofloxacin, moxifloxacin, meropenem, or ciprofloxacin. In some embodiments, the antibiotic is ciprofloxacin. In some embodiments, the antibiotic is ciprofloxacin and pharmaceutically acceptable salts thereof. In some embodiments, the antibiotic is ciprofloxacin hydrochloride. In some embodiments, the antibiotic is levofloxacin. In some embodiments, the antibiotic is ceftriaxone. In some embodiments, the antibiotic is ciprofloxacin, gatifloxacin, ceftriaxone, gemifloxacin, moxalactam, levofloxacin, meropenem, or ampicillin; or pharmaceutically acceptable salts thereof. In some embodiments, the antibiotic is ciprofloxacin, gatifloxacin, ceftriaxone, gemifloxacin, moxalactam, levofloxacin, meropenem, or ampicillin. In some embodiments, the antibiotic (e.g., fluoroquinolone or a beta lactam antibiotic, for example, ciprofloxacin, ceftriaxone) is formulated in the composition from a powder form of the antibiotic. In some embodiments, the antibiotic (e.g., fluoroquinolone or a beta lactam antibiotic, for example, ciprofloxacin, ceftriaxone) is formulated in the composition from a liquid form of the antibiotic.

Exemplary antibiotics, include, but are not limited to: Abamectin, Actinomycin (e.g., Actinomycin A, Actinomycin C, Actinomycin D, Aurantin), Alatrofloxacin mesylate, Amikacin sulfate, Aminosalicylic acid, Anthracyclines (e.g., Aclarubicin, Adriamycin, Doxorubicin, Epirubicin, Idarubicin), Antimycin (e.g., Antimycin A), Avermectin, BAL 30072, Bacitracin, Bleomycin, Cephalosporins (e.g., 7-Aminocephalosporanic acid, 7-Aminodeacetoxycephalosporanic acid, Cefaclor, Cefadroxil, Cefamandole, Cefazolin, Cefepime, Cefixime, Cefmenoxime, Cefmetazole, Cefoperazone, Cefotaxime, Cefotetan, Cefotiam, Cefoxitin, Cefpirome, Cefpodoxime proxetil, Cefsulodin, Cefsulodin sodium, Ceftazidime, Ceftizoxime, Ceftriaxone, Cefuroxime, Cephalexin, Cephaloridine, Cephalosporin C, Cephalothin, Cephalothin sodium, Cephapirin, Cephradine), Ciprofloxacin, Enrofloxacin, Clarithromycin, Clavulanic acid, Clindamycin, Colicin, Cyclosporin (e.g. Cyclosporin A), Dalfopristin/quinupristin, Daunorubicin, Doxorubicin, Epirubicin, GSK 1322322, Geneticin, Gentamicin, Gentamicin sulfate, Gramicidin (e.g. Gramicidin A), Grepafloxacin hydrochloride, Ivermectin, Kanamycin (e.g. Kanamycin A), Lasalocid, Leucomycin, Levofloxacin, Linezolid, Lomefloxacin, Lovastatin, MK 7655, Meropenem, Mevastatin, Mithramycin, Mitomycin, Monomycin, Natamycin, Neocarzinostatin, Neomycin (e.g. Neomycin sulfate), Nystatin, Oligomycin, Olivomycin, Pefloxacin, Penicillin (e.g. 6-Aminopenicillanic acid, Amoxicillin, Amoxicillin-clavulanic acid, Ampicillin, Ampicillin sodium, Azlocillin, Carbenicillin, Cefoxitin, Cephaloridine, Cloxacillin, Dicloxacillin, Mecillinam, Methicillin, Mezlocillin, Nafcillin, Oxacillin, Penicillin G, Penicillin G potassium, Penicillin G procaine, Penicillin G sodium, Penicillin V, Piperacillin, Piperacillin-tazobactam, Sulbactam, Tazobactam, Ticarcillin), Phleomycin, Polymyxin (e.g., Colistin, Polymyxin B), Pyocin (e.g. Pyocin R), RPX 7009, Rapamycin, Ristocetin, Salinomycin, Sparfloxacin, Spectinomycin, Spiramycin, Streptogramin, Streptovaricin, Tedizolid phosphate, Teicoplanin, Telithromycin, Tetracyclines (e.g. Achromycin V, Demeclocycline, Doxycycline, Doxycycline monohydrate, Minocycline, Oxytetracycline, Oxytetracycline hydrochloride Tetracycline, Tetracycline hydrochloride), Trichostatin A, Trovafloxacin, Tunicamycin, Tyrocidine, Valinomycin, (−)-Florfenicol, Acetylsulfisoxazole, Actinonin, Amikacin sulfate, Benzethonium chloride, Cetrimide, Chelerythrine, Chlorhexidine (e.g., Chlorhexidine gluconate), Chlorhexidine acetate, Chlorhexidine gluconate, Chlorothalonil, Co-Trimoxazole, Dichlorophene, Didecyldimethylammonium chloride, Dihydrostreptomycin, Enoxacin, Ethambutol, Fleroxacin, Furazolidone, Methylisothiazolinone, Monolaurin, Oxolinic acid, Povidone-iodine, Spirocheticides (e.g., Arsphenamine, Neoarsphenamine), Sulfaquinoxaline, Thiamphenicol, Tinidazole, Triclosan, Trovafloxacin, Tuberculostatics (e.g., 4-Aminosalicylic acid, AZD 5847, Aminosalicylic acid, Ethionamide), Vidarabine, Zinc pyrithione, and Zirconium phosphate.

In certain embodiments, the therapeutic agent is a Food and Drug Administration (FDA) approved drug for treating infections or infectious diseases. Exemplary FDA approved agents include, but are not limited to: Avycaz (ceftazidime-avibactam), Cresemba (isavuconazonium sulfate), Evotaz (atazanavir and cobicistat, Prezcobix (darunavir and cobicistat), Dalvance (dalbavancin), Harvoni (ledipasvir and sofosbuvir), Impavido (miltefosine), Jublia (efinaconazole), Kerydin (tavaborole), Metronidazole, Orbactiv (oritavancin), Rapivab (peramivir injection), Sivextro (tedizolid phosphate), Triumeq (abacavir, dolutegravir, and lamivudine), Viekira Pak (ombitasvir, paritaprevir, ritonavir and dasabuvir), Xtoro (finafloxacin), Zerbaxa (ceftolozane+tazobactam), Luzu (luliconazole), Olysio (simeprevir), Sitavig (acyclovir), Sovaldi (sofosbuvir), Abthrax (raxibacumab), Afinitor (everolimus), Cystaran (cysteamine hydrochloride), Dymista (azelastine hydrochloride and fluticasone propionate), Fulyzaq (crofelemer), Jetrea (ocriplasmin), Linzess (linaclotide), Qnasl (beclomethasone dipropionate) nasal aerosol, Sirturo (bedaquiline), Sklice (ivermectin), Stribild (elvitegravir, cobicistat, emtricitabine, tenofovir disoproxil fumarate), Tudorza Pressair (aclidinium bromide inhalation powder), Complera (emtricitabine/rilpivirine/tenofovir disoproxil fumarate), Dificid (fidaxomicin), Edurant (rilpivirine), Eylea (aflibercept), Firazyr (icatibant), Gralise (gabapentin), Incivek (telaprevir), Victrelis (boceprevir), Egrifta (tesamorelin), Teflaro (ceftaroline fosamil), Zymaxid (gatifloxacin), Bepreve (bepotastine besilate), Vibativ (telavancin), Aptivus (tipranavir), Astepro (azelastine hydrochloride nasal spray), Intelence (etravirine), Patanase (olopatadine hydrochloride), Viread (tenofovir disoproxil fumarate), Isentress (raltegravir), Selzentry (maraviroc), Veramyst (fluticasone furoate), Xyzal (levocetirizine dihydrochloride), Eraxis (anidulafungin), Noxafil (posaconazole), Prezista (darunavir), Tyzeka (telbivudine), Veregen (kunecatechins), Baraclude (entecavir), Fuzeon (enfuvirtide), Lexiva (fosamprenavir calcium), Reyataz (atazanavir sulfate), Clarinex, Hepsera (adefovir dipivoxil), Pegasys (peginterferon alfa-2a), Sustiva, Vfend (voriconazole), Zelnorm (tegaserod maleate), Avelox (moxifloxacin hydrochloride), Cancidas, Invanz, Peg-Intron (peginterferon alfa-2b), Rebetol (ribavirin), Spectracef, Tavist (clemastine fumarate), Twinrix, Valcyte (valganciclovir HCl), Xigris (drotrecogin alfa), ABREVA (docosanol), Cefazolin, Kaletra, Lamisil (terbinafine hydrochloride), Lotrisone (clotrimazole/betamethasone diproprionate), Lotronex (alosetron HCL), Trizivir (abacavir sulfate, lamivudine, zidovudine AZT), Synercid, Synagis, Viroptic, Aldara (imiquimod), Bactroban, Ceftin (cefuroxime axetil), Combivir, Condylox (pokofilox), Famvir (famciclovir), Floxin, Fortovase, INFERGEN (interferon alfacon-1), Intron A (interferon alfa-2b, recombinant), Mentax (butenafine HCl), Norvir (ritonavir), Omnicef, Rescriptor (delavirdine mesylate), Taxol, Timentin, Trovan, VIRACEPT (nelfinavir mesylate), Zerit (stavudine), AK-Con-A (naphazoline ophthalmic), Allegra (fexofenadine hydrochloride), Astelin nasal spray, Atrovent (ipratropium bromide), Augmentin (amoxicillin/clavulanate), Crixivan (Indinavir sulfate), Elmiron (pentosan polysulfate sodium), Havrix, Leukine (sargramostim), Merrem (meropenem), Nasacort AQ (triamcinolone acetonide), Tavist (clemastine fumarate), Vancenase AQ, Videx (didanosine), Viramune (nevirapine), Zithromax (azithromycin), Cedax (ceftibuten), Clarithromycin (Biaxin), Epivir (lamivudine), Invirase (saquinavir), Valtrex (valacyclovir HCl), Zyrtec (cetirizine HCl), Acyclovir, Penicillin (penicillin g potassium), Cubicin (Daptomycin), Factive (Gemifloxacin), Albenza (albendazole), Alinia (nitazoxanide), Altabax (retapamulin), AzaSite (azithromycin), Besivance (besifloxacin ophthalmic suspension), Biaxin XL (clarithromycin extended-release), Cayston (aztreonam), Cleocin (clindamycin phosphate), Doribax (doripenem), Dynabac, Flagyl ER, Ketek (telithromycin), Moxatag (amoxicillin), Rapamune (sirolimus), Restasis (cyclosporine), Tindamax (tinidazole), Tygacil (tigecycline), and Xifaxan (rifaximin). In certain embodiments, the antibiotic agent is selected from the group consisting of ciprofloxacin, cefuroxime, cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftobiprole, enoxacin, gatifloxacin, levofloxacin, lomefloxacin, moxifloxacin, norfloxacin, ofloxacin, trovafloxacin, bacitracin, colistin, polymyxin B, azithromycin, clarithromycin, dirithromycin, erythromycin, roxithromycin, troleandomycin, telithromycin, spectinomycin, amoxicillin, ampicillin, azlocillin, carbenicillin, cloxacillin, dicloxacillin, flucloxacillin, mezlocillin, meticillin, nafcillin, oxacillin, penicillin, piperacillin, ticarcillin, mafenide, sulfacetamide, sulfamethizole, sulfasalazine, sulfisoxazole, trimethoprim, and trimethoprim-sulfamethoxazole. In certain embodiments, the antibiotic agent is ciprofloxacin, gatifloxacin, ceftriaxone, gemifloxacin, moxalactam, levofloxacin, meropenem, or ampicillin. In certain embodiments, the antibiotic agent is ciprofloxacin or ceftriaxone. In certain embodiments, the antibiotic agent is ciprofloxacin. In certain embodiments, the antibiotic agent is ceftriaxone.

In certain embodiments, the therapeutic agent is a local anesthetic (e.g., tetrodotoxin, saxitoxin, neosaxitoxin, bupivacaine, amylocaine, ambucaine, articaine, benzocaine, benzonatate, butacaine, butanilicaine, carbocaine, cepastat, chloraseptic, chloroprocaine, cinchocaine, citanest, cyclomethycaine, dibucaine, diperodon, dimethocaine, eucaine, etidocaine, fomocaine, fotocaine, hydroxyprocaine, isobucaine, levobupivacaine, lidocaine, marcaine, mepivacaine, meprylcaine, metabutoxycaine, nitracaine, orthocaine, orabloc, oxetacaine, oxybuprocaine, paraethoxycaine, phenacaine, piperocaine, piridocaine, polocaine, posimir, pramocaine, prilocaine, primacaine, procaine, procainamide, proparacaine, propoxycaine, pyrrocaine, quinisocaine, ropivacaine, sensorcaine, septocaine, trimecaine, tetracaine, tolycaine, tropacocaine, ulcerease, xylocaine, or zorcaine). In certain embodiments, the therapeutic agent is a local anesthetic compound, for example, but not limited to, an amino ester compound (e.g., procaine, tetracaine, chloroprocaine, benzocaine, butacaine, dimethocaine) or an amino amide compound (e.g., procainamide, lidocaine). In certain embodiments, the local anesthetic is a sodium channel blocker, for example, a site 1 sodium channel blocker (e.g., tetrodotoxin, saxitoxins (saxitoxin, neosaxitoxin), gonyautoxins (gonyautoxin V, gonyautoxin VI), μ-conotoxins) or an amino amide local anesthetic. In certain embodiments, the local anesthetic is tetrodotoxin, saxitoxin, neosaxitoxin, bupivacaine, amylocaine, ambucaine, articaine, benzocaine, benzonatate, butacaine, butanilicaine, carbocaine, cepastat, chloraseptic, chloroprocaine, cinchocaine, citanest, cyclomethycaine, dibucaine, diperodon, dimethocaine, eucaine, etidocaine, fomocaine, fotocaine, hydroxyprocaine, isobucaine, levobupivacaine, lidocaine, marcaine, mepivacaine, meprylcaine, metabutoxycaine, nitracaine, orthocaine, orabloc, oxetacaine, oxybuprocaine, paraethoxycaine, phenacaine, piperocaine, piridocaine, polocaine, posimir, pramocaine, prilocaine, primacaine, procaine, procainamide, proparacaine, propoxycaine, pyrrocaine, quinisocaine, ropivacaine, sensorcaine, septocaine, trimecaine, tetracaine, tolycaine, tropacocaine, ulcerease, xylocaine, or zorcaine. In certain embodiments, the local anesthetic is tetrodotoxin, saxitoxin, neosaxitoxin, lidocaine, procaine, or bupivacaine. In certain embodiments, the local anesthetic is bupivacaine.

In certain embodiments, the agent is an immunostimulatory agent, for example, an immunostimulatory agent is an agent that stimulates an immune response (including enhancing a pre-existing immune response) in a subject to whom it is administered, whether alone or in combination with another agent. Examples include antigens, adjuvants (e.g., TLR ligands such as imiquimod and residuimod, imidazoquino lines, nucleic acids comprising an unmethylated CpG dinucleotide, monophosphoryl lipid A (MPLA) or other lipopolysaccharide derivatives, single-stranded or double Stranded RNA, flagellin, muramyl dipeptide), cytokines including interleukins (e.g., IL-2, IL-7, IL-15 (or superagonist/mutant forms of these cytokines), IL-12, IFN-gamma, IFN-alpha, GM-CSF, FLT3-ligand, etc.), immunostimulatory antibodies (e.g., anti-CTLA-4, anti-CD28, anti-CD3, or single chain/antibody fragments of these molecules), and the like.

In certain embodiments, the agent is an antigen. The antigen may be without limitation a cancer antigen, a self or autoimmune antigen, a microbial antigen, an allergen, or an environmental antigen. The antigen may be peptide, lipid, or carbohydrate in nature, but it is not so limited. In certain embodiments, the antigen agent is a cancer antigen. A cancer antigen is an antigen that is expressed preferentially by cancer cells (i.e., it is expressed at higher levels in cancer cells than on non-cancer cells) and in Some instances it is expressed solely by cancer cells. The cancer antigen may be expressed within a cancer cell or on the surface of the cancer cell. The cancer antigen may be MART-1/Melan-A, gp100, adenosine deaminase-binding protein (ADAbp), FAP, cyclophilin b, colorectal associated antigen (CRC)—C0171A/GAT33, carcinoembryonic antigen (CEA), CAP-1, CAP2, etv6, AMLI, prostate specific antigen (PSA), PSA-1, PSA2, PSA-3, prostate-specific membrane antigen (PSMA), T cell receptor/CD3-Zeta chain, and CD20. The cancer antigen may be selected from the group consisting of MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5). The cancer antigen may be selected from the group consisting of GAGE-1, GAGE-2, GAGE-3, GAGE-4, GAGE-5, GAGE-6, GAGE-7, GAGE-8, GAGE-9. The cancer antigen may be selected from the group consisting of BAGE, RAGE, LAGE-1, NAG, GnTV. MUM-1, CDK4, tyrosinase, p53, MUC family, HER2/neu, p21 ras, RCAS1, C.-fetoprotein, E-cadherin, C-catenin, B-catenin, Y-catenin, p120ctn, gp100′″7, PRAME, NY-ESO-1, cdc27, adenomatous polyposis coli protein (APC), fodrin, Connexin 37. Ig-idiotype, p15, gp75, GM2 ganglioside, GD2 ganglioside, human papilloma virus proteins, Smad family of tumor antigens, Imp-1, PlA, EBV-encoded nuclear antigen (EBNA)-1, brain glycogen phosphorylase, SSX-1, SSX-2(HOM-MEL-40), SSX-1, SSX-4, SSX-5, SCP-1 and CT-7, CD20, and c-erbB-2.

In certain embodiments, the agent is an anti-cancer agent. An anti-cancer agent is an agent that at least partially inhibits the development or progression of a cancer, including inhibiting in whole or in part symptoms associated with the cancer even if only for the short term. Several anti-cancer agents can be categorized as DNA damaging agents and these include topoisomerase inhibitors, DNA alkylating agents, DNA strand break inducing agents, anti-microtubule agents, anti-metabolic agents, anthracyclines, Vinca alkaloids, or epipodophyllotoxins. The anti-cancer agent may be an enzyme inhibitor including without limitation tyrosine kinase inhibitor, a CDK inhibitor, a MAP kinase inhibitor, or an EGFR inhibitor. The anti-cancer agent may be a VEGF inhibitor including without limitation bevacizumab (AVASTIN), ranibizumab (LUCENTIS), pegaptanib (MACUGEN), Sorafenib, sunitinib (SUTENT), vatalanib, ZD-6474 (ZACTIMA), anecortave (RETAANE), squalamine lactate, and semaphorin. The anti-cancer agent may be an antibody or an antibody fragment including without limitation an antibody or an anti body fragment including but not limited to bevacizumab (AVASTIN), trastuzumab (HERCEPTIN), alemtuzumab (CAMPATH, indicated for B cell chronic lymphocytic leukemia.), gemtuzumab (MYLOTARG, hP67.6, anti-CD33, indicated for leukemia Such as acute myeloid leukemia), rituximab (RITUXAN), to situmomab (BEXXAR, anti-CD20, indicated for B cell malignancy), MDX-210 (bispecific antibody that binds simultaneously to HER-2/neu oncogene protein product and type I Fc receptors for immunoglobulin G (IgG) (Fc gamma RI)). oregovomab (OVAREX, indicated for ovarian cancer), edrecolomab (PANOREX), daclizumab (ZENAPAX), palivizumab (SYNAGIS, indicated for respiratory conditions such as RSV infection), ibritumomab tiuxetan (ZEVALIN, indicated for Non-Hodgkin's lymphoma), cetuximab (ERBITUX), MDX-447, MDX-22, MDX-220 (antiTAG-72), IOR—C5, IOR-T6 (anti-CD1), IOR EGF/R3, celogovab (ONCOSCINT OV103), epratuzumab (LYMPHOCIDE), pemtumomab (THERAGYN), and Gliomab-H (indicated for brain cancer, melanoma). In certain embodiments, the agent is doxorubicin.

In certain embodiments, the therapeutic agent is a chemotherapeutic agent or an antibiotic agent. In certain embodiments, the therapeutic agent is an anesthetic agent, chemotherapeutic agent, or an antibiotic agent. In certain embodiments, the therapeutic agent is an anesthetic agent or a chemotherapeutic agent. In certain embodiments, the therapeutic agent is doxorubicin, tetrodotoxin, saxitoxin, neosaxitoxin, bupivacaine, amylocaine, ambucaine, articaine, benzocaine, benzonatate, butacaine, butanilicaine, carbocaine, cepastat, chloraseptic, chloroprocaine, cinchocaine, citanest, cyclomethycaine, dibucaine, diperodon, dimethocaine, eucaine, etidocaine, fomocaine, fotocaine, hydroxyprocaine, isobucaine, levobupivacaine, lidocaine, marcaine, mepivacaine, meprylcaine, metabutoxycaine, nitracaine, orthocaine, orabloc, oxetacaine, oxybuprocaine, paraethoxycaine, phenacaine, piperocaine, piridocaine, polocaine, posimir, pramocaine, prilocaine, primacaine, procaine, procainamide, proparacaine, propoxycaine, pyrrocaine, quinisocaine, ropivacaine, sensorcaine, septocaine, trimecaine, tetracaine, tolycaine, tropacocaine, ulcerease, xylocaine, or zorcaine. In certain embodiments, the agent is doxorubicin, bupivacaine, or tetrodotoxin. In certain embodiments, the agent is bupivacaine or tetrodotoxin. In certain embodiments, the agent is bupivacaine. In certain embodiments, the agent is tetrodotoxin.

In certain embodiments, the diagnostic agent is an imaging agent. An imaging agent is an agent that emits signal directly or indirectly thereby allowing its detection in vivo. Imaging agents such as contrast agents and radioactive agents that can be detected using medical imaging techniques, such as nuclear medicine scans, magnetic resonance imaging (MRI), positron-emission tomography (PET), and in vivo fluorescence imaging. Examples of such imaging agents are fluorophores (e.g., Cy7, fluorescein, rhodamine, AMCA), or biological fluorophores (e.g., green fluorescent protein, phycoerythrin, allophycocyanin). In certain embodiments, the diagnostic agent is Sulforhodamine B, indocyanine green, fluorescein isothiocyanate, methylene blue, or coumarin. In certain embodiments, the diagnostic agent is Sulforhodamine B, indocyanine green, methylene blue, or coumarin. In certain embodiments, the diagnostic agent is conjugated to a protein, a polymer, or a small molecule. In certain embodiments, the diagnostic agent is conjugated to a protein (e.g., albumin). In certain embodiments, the diagnostic agent is conjugated to albumin. In certain embodiments, the diagnostic agent is fluorescein isothiocyanate conjugated to albumin. In certain embodiments, the diagnostic agent is conjugated to a polymer (e.g., PEG). In certain embodiments, the diagnostic agent is conjugated to PEG having a molecular weight of about 1000 g/mol. In certain embodiments, the diagnostic agent is conjugated to PEG having a molecular weight of about 10000 g/mol. In certain embodiments, the diagnostic agent is Sulforhodamine B conjugated to PEG having a molecular weight of about 1000 g/mol. In certain embodiments, the diagnostic agent is Sulforhodamine B conjugated to PEG having a molecular weight of about 10000 g/mol.

In certain embodiments, the composition comprises at least two types of therapeutic agents selected from the group consisting of a local anesthetic (e.g., tetrodotoxin or “TTX”)), an anti-inflammatory agent, and a sympathomimetic or vasoconstrictor agent (such as alpha- and beta-adrenergic agonists (sympathomimetic or vasoconstrictor agent), for example, epinephrine; vasopressin analogs, norepinephrine, phenylephrine, dopamine, dobutamine). In certain embodiments, the composition comprises an anti-inflammatory agent (e.g., glucocorticoid receptor agonist, such as dexamethasone, corticosterone, glucocorticoids, mifepristone, hydrocortisone, corticotropin) and/or and a sympathomimetic or vasoconstrictor agent (e.g., epinephrine). In certain embodiments, the one or more agents (e.g., local anesthetic; anti-inflammatory (e.g., glucocorticoid receptor agonist, such as dexamethasone, corticosterone, glucocorticoids, mifepristone, hydrocortisone, corticotropin)) in the particle (e.g., liposome) is delivered together with one or more compounds or therapeutic agents (e.g., alpha- and beta-adrenergic agonists (sympathomimetic or vasoconstrictor agent), for example, epinephrine; vasopressin analogs, norepinephrine, phenylephrine, dopamine, dobutamine) in an injectate (liquid vehicle). In certain embodiments, in the composition, the injectate comprises an adjuvant agent such as alpha- and beta-adrenergic agonists (sympathomimetic or vasoconstrictor agent), for example, epinephrine; vasopressin analogs, norepinephrine, phenylephrine, dopamine, dobutamine); and the particle (e.g. liposome) comprises the anti-inflammatory (e.g., glucocorticoid receptor agonist, such as dexamethasone, corticosterone, glucocorticoids, mifepristone, hydrocortisone, corticotropin), and/or the local anesthetic (e.g., tetrodotoxin or “TTX”). In certain embodiments, the one or more agents (e.g., anti-inflammatory (e.g., glucocorticoid receptor agonist, such as dexamethasone, corticosterone, glucocorticoids, mifepristone, hydrocortisone, corticotropin)) in the particle is delivered together with one or more compounds or therapeutic agents (e.g., alpha- and beta-adrenergic agonists (sympathomimetic or vasoconstrictor agent), for example, epinephrine; vasopressin analogs, norepinephrine, phenylephrine, dopamine, dobutamine) in an injectate (liquid vehicle). In certain embodiments, the one or more agents (e.g., local anesthetic; anti-inflammatory (e.g., glucocorticoid receptor agonist, such as dexamethasone, corticosterone, glucocorticoids, mifepristone, hydrocortisone, corticotropin)) in the particle is delivered together with one or more compounds (e.g., alpha- and beta-adrenergic agonists (sympathomimetic or vasoconstrictor agent), for example, epinephrine; vasopressin analogs, norepinephrine, phenylephrine, dopamine, dobutamine) in an injectate (liquid vehicle), for prolonging a nerve blockade. In certain embodiments, an anti-inflammatory (e.g., glucocorticoid receptor agonist, such as dexamethasone, corticosterone, glucocorticoids, mifepristone, hydrocortisone, corticotropin) in the particle is delivered together with one or more alpha- and beta-adrenergic agonists (sympathomimetic or vasoconstrictor agent), e.g., epinephrine; vasopressin analogs, norepinephrine, phenylephrine, dopamine, dobutamine) in an injectate (liquid vehicle), for prolonging a nerve blockade. In certain embodiments, the composition comprises dexamethasone and a local anesthetic (e.g., TTX) in the particle; and epinephrine in an injectate. In certain embodiments, the composition comprises dexamethasone in the particle; and epinephrine in an injectate. In certain embodiments, the composition comprises tetrodotoxin and epinephrine in a particle and dexamethasone in another particle. In certain embodiments, the composition comprises dexamethasone in a liposome; and tetrodotoxin and epinephrine in an injectate. In certain embodiments, the composition comprises tetrodotoxin in a liposome; and epinephrine in an injectate. In certain embodiments, the composition comprises tetrodotoxin in a liposome; and epinephrine in a liposome.

In certain embodiments, the agent is a therapeutic agent, which is a small molecule therapeutic agent, a protein therapeutic agent, a nucleic acid therapeutic agent. In certain embodiments, the agent is a small molecule therapeutic agent, a protein therapeutic agent, a nucleic acid therapeutic agent, or small molecule diagnostic agent (e.g., fluorophore). In certain embodiments, the diagnostic agent is a fluorophore, for example, organic dyes (e.g., fluorescein, rhodamine, AMCA), or biological fluorophores (e.g., green fluorescent protein, phycoerythrin, allophycocyanin), including hydrophobic dyes and/or photosensitizers comprising aromatic groups. In certain embodiments, the diagnostic agent is a fluorophore, for example, organic dyes (e.g., fluorescein, rhodamine, AMCA), or biological fluorophores (e.g., green fluorescent protein, phycoerythrin, allophycocyanin). In certain embodiments, the diagnostic agent is Sulforhodamine B, indocyanine green, methylene blue, or coumarin. In certain embodiments, the diagnostic agent is Sulforhodamine B, indocyanine green, fluorescein isothiocyanate, methylene blue, or coumarin. In certain embodiments, the agent is physically incorporated into the particle. In certain embodiments, the agent is not physically incorporated into the particle. In certain embodiments, the agent is conjugated as part of Formula (I), as described herein.

In certain embodiments, the composition described herein further comprises water. In certain embodiments, the composition described herein forms a particle (e.g., liposome, lipid nanoparticle, polymer-lipid hybrid nanoparticle, lipid micelle, micelle-hydrogel hybrid, liposome-entrapped hydrogel, lipid-peptide complex, lipid-nucleic acid complex, lipid-peptide-nucleic acid complex, or lipid coated inorganic nanoparticle) upon addition of water (hydration). In certain embodiments, the composition described herein is in the form of a particle (e.g., liposome, nanoparticle, for example, lipid nanoparticle, polymer-lipid hybrid nanoparticle, lipid coated inorganic nanoparticle). In certain embodiments, the composition described herein is in the form of a liposome or nanoparticle (e.g., lipid nanoparticle, polymer-lipid hybrid nanoparticle, or lipid coated inorganic nanoparticle). In certain embodiments, the particle is a liposome. In certain embodiments, the liposome comprises dipalmitoylphosphatidylcholine. In certain embodiments, the particle is a liposome, lipid nanoparticle, polymer-lipid hybrid nanoparticle, lipid micelle, micelle-hydrogel hybrid, liposome-entrapped hydrogel, lipid-peptide complex, lipid-nucleic acid complex, lipid-peptide-nucleic acid complex, or lipid coated inorganic nanoparticle. In certain embodiments, the particle is a liposome, lipid nanoparticle, polymer-lipid hybrid nanoparticle, or lipid coated inorganic nanoparticle.

### Pharmaceutical Compositions, Kits, and Administration

In certain embodiments, provided herein is a pharmaceutical composition comprising a composition described herein, a therapeutic agent described herein, and optionally a pharmaceutically acceptable excipient. As used herein, the term “excipient” means a non-toxic, inert solid, semi-solid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The compositions of this invention can be administered to subjects (e.g., humans and/or to animals), orally, rectally, parenterally, intracisternally, intravaginally, intranasally, intraperitoneally, topically (as by powders, creams, ointments, or drops), bucally, or as an oral or nasal spray.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the cell being contacted with a compound or composition described herein is in vitro. In certain embodiments, the cell being contacted with a compound or composition described herein is in vivo.

Liquid dosage forms for oral administration include emulsions, microemulsions, solutions, suspensions, syrups, and elixirs. In addition to the active ingredients (i.e., microparticles, nanoparticles, liposomes, micelles, polynucleotide/lipid complexes), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80.

The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration are preferably suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid dosage forms for oral administration include capsules, tablets, pills, powders, and granules. In such solid dosage forms, the particles are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Solid compositions of a similar type may also be employed as fillers in soft and hard-filled gelatin capsules using such excipients as lactose or milk sugar as well as high molecular weight polyethylene glycols and the like.

Dosage forms for topical or transdermal administration of an inventive pharmaceutical composition include ointments, pastes, creams, lotions, gels, powders, solutions, sprays, inhalants, or patches. The particles are admixed under sterile conditions with a pharmaceutically acceptable carrier and any needed preservatives or buffers as may be required. Ophthalmic formulation, ear drops, and eye drops are also contemplated as being within the scope of this invention.

The ointments, pastes, creams, and gels may contain, in addition to the particles of this invention, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the particles of this invention, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the microparticles or nanoparticles in a proper medium. Absorption enhancers can also be used to increase the flux of the compound across the skin. The rate can be controlled by either providing a rate controlling membrane or by dispersing the particles in a polymer matrix or gel.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a biological sample, tissue, or cell, any two doses of the multiple doses include different or substantially the same amounts of a compound described herein. In certain embodiments, when multiple doses are administered to a subject or applied to a biological sample, tissue, or cell, the frequency of administering the multiple doses to the subject or applying the multiple doses to the biological sample, tissue, or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the biological sample, tissue, or cell is one dose per day. In certain embodiments, the frequency of administering the multiple doses to the subject or applying the multiple doses to the biological sample, tissue, or cell is two doses per day. In certain embodiments, the frequency of administering the multiple doses to the subject or applying the multiple doses to the biological sample, tissue, or cell is three doses per day. In certain embodiments, when multiple doses are administered to a subject or applied to a biological sample, tissue, or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, or biological sample (e.g., tissue, or cell). In certain embodiments, the duration between the first dose and last dose of the multiple doses is three months, six months, or one year. In certain embodiments, the duration between the first dose and last dose of the multiple doses is the lifetime of the subject or biological sample (e.g., tissue, or cell). In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein. In certain embodiments, the therapeutic agent, for example, local anesthetic (e.g., TTX) comprises between approximately 0-100 μg therapeutic agent, (0-0.1% in mass percentage of the formulations) per 0.45 kg rat, or an equivalent amount in a human dosage, for example, between approximately 0 to 1.0 mg/kg human (e.g., 0-0.1 mg/kg, 0.1-0.2 mg/kg, 0.2-0.3 mg/kg, 0.2-0.3 mg/kg, 0.3-0.4 mg/kg, 0.4-0.5 mg/kg, 0.5-0.6 mg/kg, 0.6-0.7 mg/kg, 0.8-0.9 mg/kg, 0.9-1.0 mg/kg, 1.0-1.2 mg/kg). Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult. The amount to be administered to, for example, a human, can be calculated from the amount administered to a rat, as determined using standard calculations by a person of ordinary skill in the art.

### Kits

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound, composition, or pharmaceutical composition described herein. In certain embodiments, the kits are useful for delivering an agent to a subject, comprising a composition described herein, the agent, and instructions for delivering the agent to a subject in need thereof. In certain embodiments, the kits comprise a container, a composition of a compound described herein, and instructions for administering the composition or pharmaceutical composition thereof to a subject in need thereof. In certain embodiments, the kits comprise a container, a composition of a compound described herein, and instructions for administering the composition or pharmaceutical composition thereof to a subject in need thereof. In certain embodiments, a kit described herein further includes instructions for using the compound or pharmaceutical composition included in the kit. A kit described herein may also include information as required by a regulatory agency such as the U.S. Food and Drug Administration (FDA). In certain embodiments, the information included in the kits is prescribing information. In certain embodiments, the kits and instructions provide for assembling the composition described herein. A kit described herein may include one or more additional pharmaceutical agents described herein as a separate composition. In certain embodiments, a kit described herein further includes a dropper, syringe, or catheter. In certain embodiments, a kit described herein further includes a syringe.

### Methods of Treatment and Uses

In one aspect, provided are methods for delivering an agent described herein (e.g., therapeutic agent (such as antibiotic agent, chemotherapeutic agent, anesthetic agent, anti-inflammatory agent, analgesic agent, anti-fibrotic agent, anti-sclerotic agent, or anticoagulant agent) or diagnostic agent, such as a fluorophore, e.g., Sulforhodamine B, indocyanine green, methylene blue, coumarin) to a subject or biological sample (e.g., cell, tissue), comprising administering to the subject or contacting the biological sample with a composition described herein (e.g., composition comprising a compound of Formula (I), or administering to the subject or contacting the biological sample with the pharmaceutical composition described herein. In certain embodiments, the composition is in the form of a particle, e.g., a liposome, lipid nanoparticle, polymer-lipid hybrid nanoparticle, lipid micelle, micelle-hydrogel hybrid, liposome-entrapped hydrogel, lipid-peptide complex, lipid-nucleic acid complex, lipid-peptide-nucleic acid complex, or lipid coated inorganic nanoparticle. In certain embodiments, the composition is in the form of a particle, e.g., a liposome, lipid nanoparticle, polymer-lipid hybrid nanoparticle, or lipid coated inorganic nanoparticle. In certain embodiments, the composition is in the form of a liposome. In certain embodiments, the composition is in the form of a nanoparticle. In certain embodiments, the composition is in the form of a nanoparticle, for example, a lipid nanoparticle, polymer-lipid hybrid nanoparticle, or lipid coated inorganic nanoparticle. In certain embodiments, the methods for delivering an agent (e.g., therapeutic agent, diagnostic agent) described herein are used for local anesthesia, photodynamic therapy, inflammation, molecular imaging, photothermal therapy, and/or fluorescence imaging. In certain embodiments, the methods for delivering an anesthetic agent (e.g., local anesthetic) described herein elicit prolonged peripheral nerve blockade for up to three or more days following a single application (e.g., injection) of the composition described herein.

In certain embodiments, in methods described herein, in therapeutic applications, a drug may be incorporated into the aqueous core of the liposomes or interior of the lipid bilayers. In certain embodiments, in methods described herein, in diagnostic applications, a labeling moiety may be conjugated to the phospholipids and/or incorporated into the interior of the lipid bilayers.

For example, in one aspect, provided is a method of treating and/or preventing a disease in a subject in need thereof, the method comprising administering to the subject a composition described herein (e.g., composition comprising a compound of Formula (I) (e.g., in the form of a particle, for example, a liposome, lipid nanoparticle, polymer-lipid hybrid nanoparticle, or lipid coated inorganic nanoparticle) comprising a therapeutically effective amount of a therapeutic agent (e.g., a small molecule therapeutic agent, a protein therapeutic agent, a nucleic acid therapeutic agent; for example, antibiotic agent, chemotherapeutic agent, anesthetic agent, anti-inflammatory agent, analgesic agent, anti-fibrotic agent, anti-sclerotic agent, anticoagulant agent), or a pharmaceutical composition described herein.

The present disclosure also provides uses of a composition described herein (e.g., composition comprising a compound of Formula (I), for delivering an agent described herein (e.g., therapeutic agent (such as antibiotic agent, chemotherapeutic agent, anesthetic agent, anti-inflammatory agent, analgesic agent, anti-fibrotic agent, anti-sclerotic agent, or anticoagulant agent) or diagnostic agent, such as a fluorophore, e.g., Sulforhodamine B, indocyanine green, methylene blue, coumarin) to a subject or biological sample (e.g., cell, tissue), comprising administering to the subject or contacting the biological sample with a composition described herein (e.g., composition comprising a compound of Formula (I), or administering to the subject or contacting the biological sample with the pharmaceutical composition described herein. In certain embodiments, the composition is in the form of a particle, e.g., a liposome, lipid nanoparticle, polymer-lipid hybrid nanoparticle, or lipid coated inorganic nanoparticle.

The present disclosure also provides uses of a composition described herein (e.g., composition comprising a compound of Formula (I), for treating and/or preventing a disease in a subject in need thereof, the method comprising administering to the subject a composition described herein (e.g., composition comprising a compound of Formula (I) (e.g., in the form of a particle, for example, a liposome, lipid nanoparticle, polymer-lipid hybrid nanoparticle, or lipid coated inorganic nanoparticle) comprising a therapeutically effective amount of a therapeutic agent (e.g., a small molecule therapeutic agent, a protein therapeutic agent, a nucleic acid therapeutic agent; for example, antibiotic agent, chemotherapeutic agent, anesthetic agent, anti-inflammatory agent, analgesic agent, anti-fibrotic agent, anti-sclerotic agent, anticoagulant agent), or a pharmaceutical composition described herein.

### Methods of Synthesis

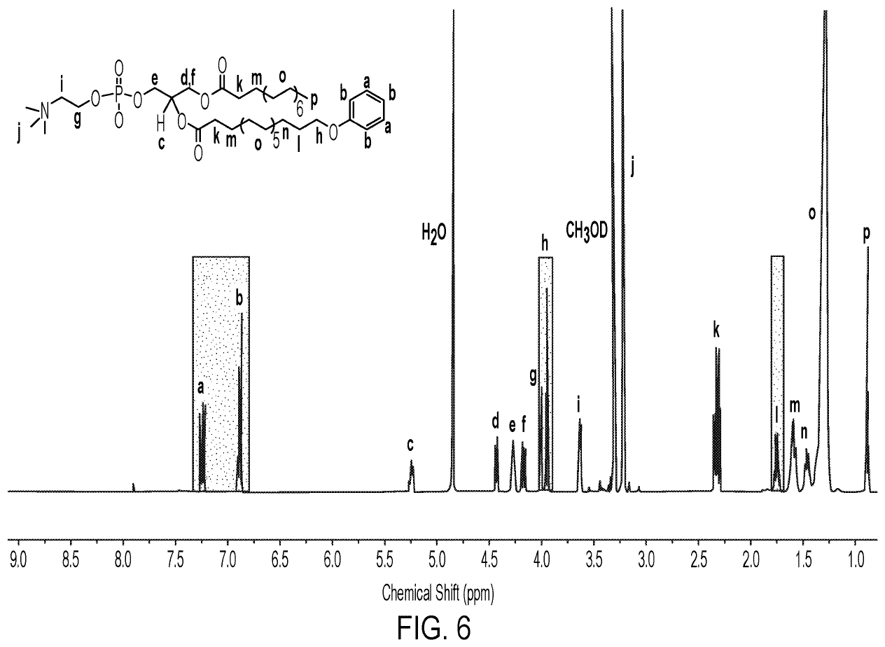

In one aspect, provided are methods of synthesizing the particles (e.g., liposomes) described herein, comprising the compounds of Formula (I) (e.g., modified phospholipids) and/or other unmodified phospholipids (e.g., phosphoglycerol, phosphocholine, phosphoethanolamine, phosphoinositol, phosphoserine; such as, DOPC, DSPC), and lipids (e.g., cholesterol) described herein, wherein the ratio of the lipids to each other is variable (e.g., ratio of 3:3:2:3). In certain embodiments, the particle (e.g., liposome) is synthesized using Ph-DPPC, DOPC, DSPG, and/or cholesterol. In certain embodiments, the particle (e.g., liposome) is synthesized using phenoxy-conjugated DPPC (“Ph-DPPC” depicted in FIG. 6), 1,2-dioleoyl-sn-glycero-3-phosphocholine (DOPC), 1,2-distearoyl-sn-glycero-3-phosphatidylglycerol (DSPG), and cholesterol, wherein the ratio of the lipids to each other is variable (e.g., ratio of 3:3:2:3).

In certain embodiments, the particle (e.g., liposome) is synthesized by combining a compound of Formula (I) (e.g., Ph-DPPC), one or more unmodified phospholipids (e.g., phosphoglycerol, phosphocholine, phosphoethanolamine, phosphoinositol, phosphoserine; such as, DOPC, DSPC), and/or lipids (e.g., cholesterol); hydrating, and mixing, which subsequently forms the particle (e.g., liposome). In certain embodiments, the particle (e.g., liposome) is synthesized by combining Ph-DPPC, DOPC, DSPG, and/or cholesterol, hydrating, and mixing, which subsequently forms the particle (e.g., liposome). In certain embodiments, the particle (e.g., liposome) is synthesized by combining Ph-DPPC, DOPC, DSPG, and cholesterol, and hydrated (e.g., with a buffer, such as phosphate buffered saline (PBS)) while mixing (e.g., agitating), which subsequently forms the particle (e.g., liposome). In certain embodiments, the particle (e.g., liposome) is synthesized by combining Ph-DPPC, DOPC, DSPG, and cholesterol, and hydrated while mixing, which subsequently forms the particle (e.g., liposome), as described in Example 1 below (“Preparation and characterization of aromatized liposomes”). In certain embodiments, the particle (e.g., liposome) is synthesized using 1-100% molar percentage (e.g., at least 10%, for example at least 15%, or at least 25%) of the compounds of Formula (I) (e.g., modified phospholipids, for example, Ph-DPPC, or a modified phospholipid of Formula (I) described herein). In certain embodiments, the particle (e.g., liposome) is synthesized using about 0-99% molar percentage (e.g., about 40-75%) of the unmodified phospholipids (e.g., phosphoglycerol, phosphocholine, phosphoethanolamine, phosphoinositol, phosphoserine; such as, DOPC, DSPC). In certain embodiments, the particle (e.g., liposome) is synthesized using 1-100% molar percentage (e.g., at least 10%, for example at least 15%, or at least 25%) of the compounds of Formula (I) (e.g., Ph-DPPC, or a modified phospholipid of Formula (I) described herein); and about 0-99% molar percentage (e.g., about 40-75%) of the unmodified phospholipids (e.g., phosphoglycerol, phosphocholine, phosphoethanolamine, phosphoinositol, phosphoserine; such as, DOPC, DSPC). In certain embodiments, provided are particles (e.g., liposomes) described herein, comprising the compounds of Formula (I) (e.g., modified phospholipids) and/or other unmodified phospholipids (e.g., phosphoglycerol, phosphocholine, phosphoethanolamine, phosphoinositol, phosphoserine; such as, DOPC, DSPC), and lipids (e.g., cholesterol) described herein, wherein the ratio of the lipids to each other is variable (e.g., ratio of 3:3:2:3), synthesized by the methods described herein. In certain embodiments, the particle (e.g., liposome) comprises an agent (e.g., therapeutic agent, diagnostic agent; e.g., TTX, Cy7) as described herein. In certain embodiments, the particle comprising unmodified phospholipids (e.g., phosphoglycerol, phosphocholine, phosphoethanolamine, phosphoinositol, phosphoserine; such as, DOPC, DSPC), and lipids (e.g., cholesterol) described herein, wherein the ratio of the lipids to each other is variable (e.g., ratio of 3:3:2:3), is as described in Example 1.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting their scope. The compounds provided herein can be prepared from readily available starting materials using the following general methods and procedures or methods known in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

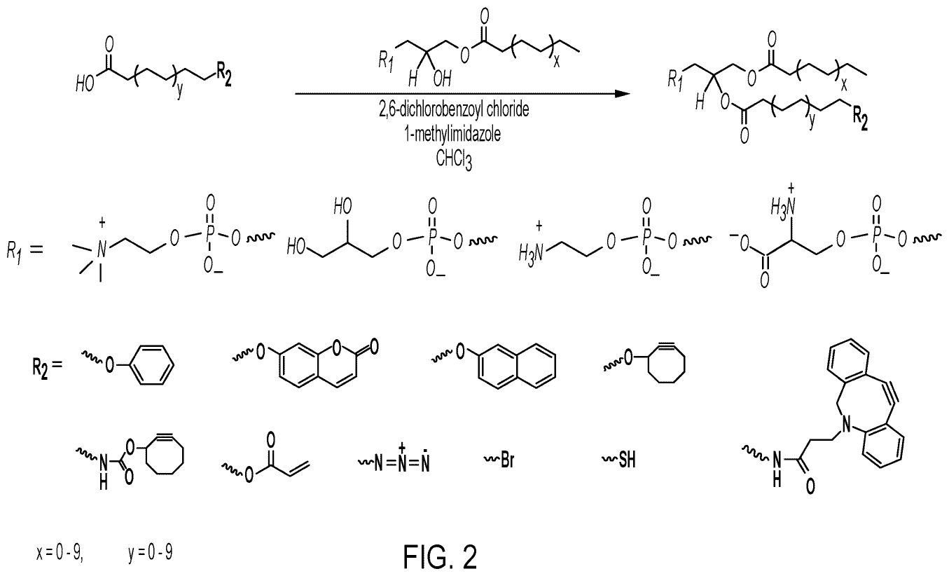

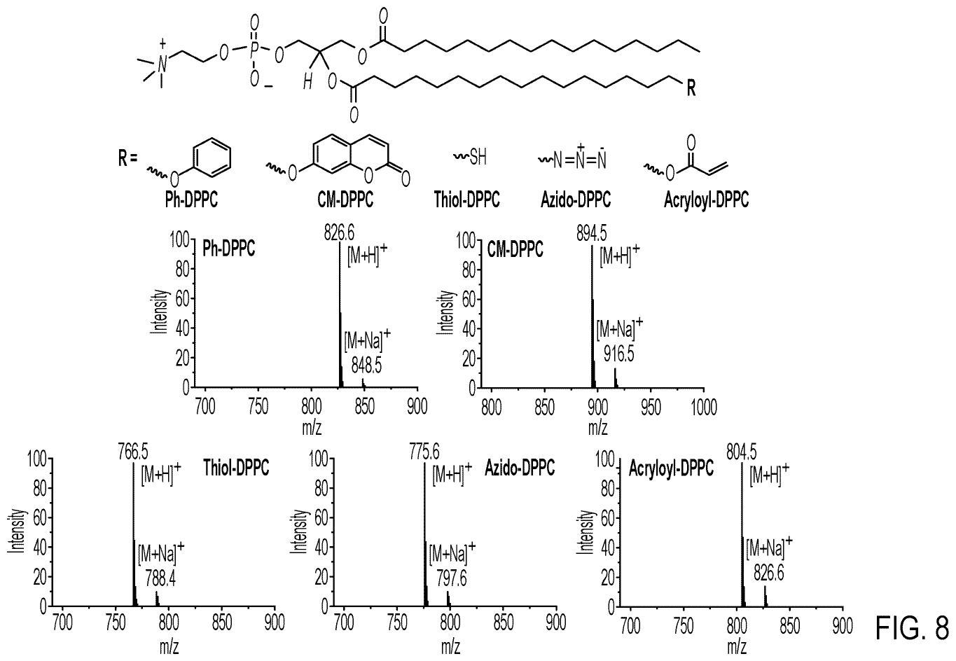

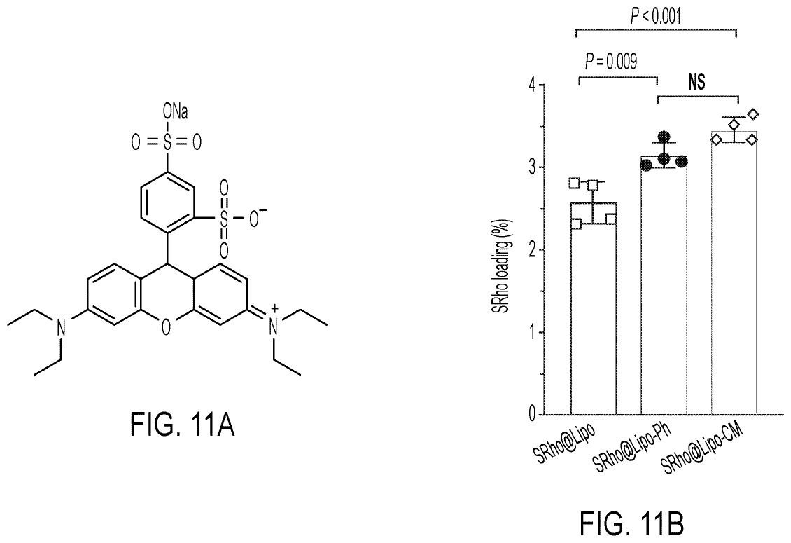

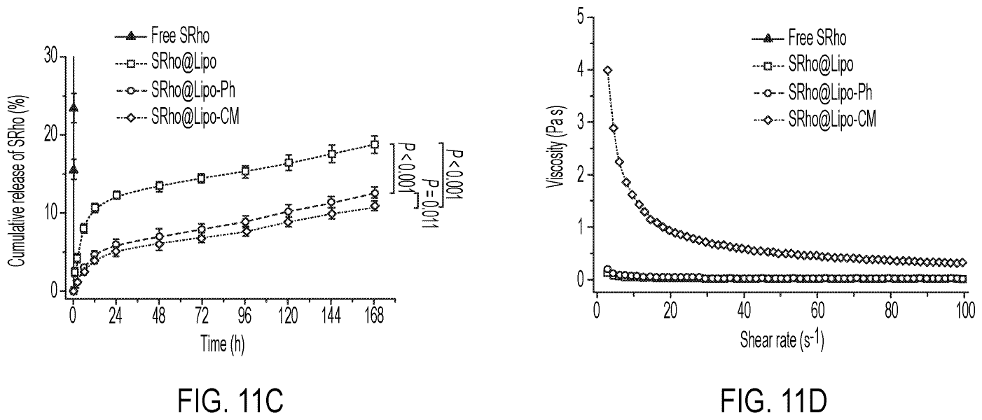

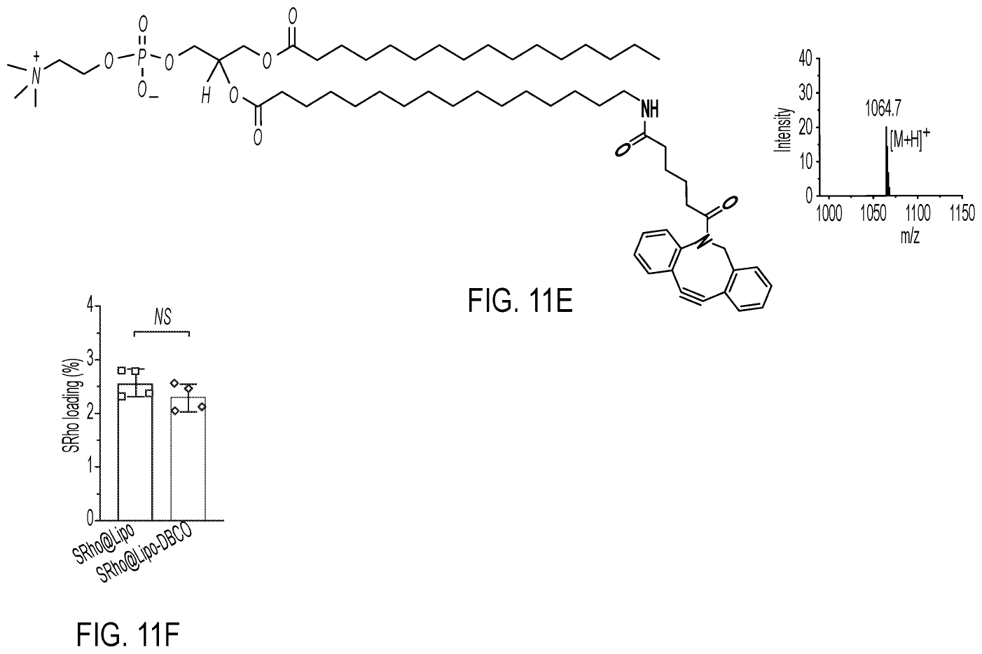

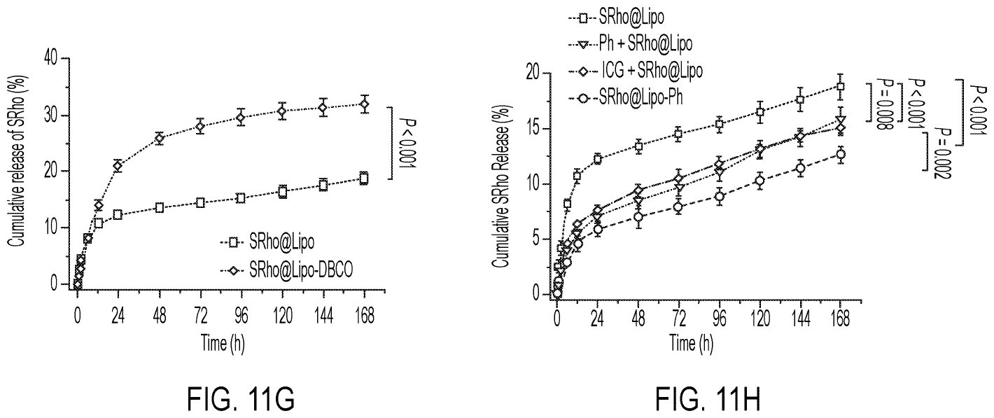

In certain embodiments, the phospholipid compounds disclosed herein are synthesized as outlined in Examples 1 and 2, and in the scheme of FIG. 2. DPPC is dipalmitoylphosphatidylcholine. In certain examples, DPPC is modified with various functional groups, e.g., Ph-DPPC is DPPC modified with a phenoxy group and CM-DPPC is DPPD modified with a coumarin group as shown in FIG. 8. “Lip-Ph” or “Lipo-Ph” are liposomes containing Ph-DPPC. “Lip-Ph” or “Lipo-Ph” are liposomes containing Ph-DPPC. “Lipo-CM” are liposomes containing CM-DPPC. “Lipo-DBCO” are liposomes containing DPPC modified with a DBCO group (e.g., FIG. 11E). Unmodified liposome is referred to as “Lip” or “Lipo”. Unmodified liposome is DPPC.

Nomenclature in the application includes naming the agent encapsulated in a liposome by use of “agent name” @liposome (e.g., TTX@Lipo-Ph is tetrodotoxin encapsulated in liposomes containing Ph-DPPC) or the agent name after the liposome (e.g., Lip-Ph-Bup is bupivacaine encapsulated in liposomes containing Ph-DPPC).

### Example 1: Acyl Chain-Modified Phospholipids and Lipids Incorporating TTX and Dyes

The engineering of synthetic lipid vesicles that incorporate aromatic groups within lipid bilayers for improved drug loading and sustained drug release, using a strategy for the synthesis of acyl chain-modified phospholipids with terminal aromatic groups are described. Formulation of phospholipids conjugated to aromatic groups and of natural phospholipids allowed the formation of aromatized liposomes which may have similar physiochemical properties to conventional liposomes but have significantly decreased membrane permeability and leakage of encapsulated cargos.

This data shows an example in an area where liposome products are used clinically: local anesthesia.² The anesthetic tetrodotoxin (TTX) was selected as the principal model compound as it is it has many of the properties that are most problematic in encapsulation in liposomes: low molecular weight, extreme hydrophilicity, high potency, and narrow therapeutic window. Aromatized liposomes enabled increased TTX loading, prolonged therapeutic duration 20-30-fold compared to free TTX and by more than 3-fold compared to unmodified liposomes, expanded the therapeutic window, and mitigated systemic toxicity. These rationally designed phospholipids could create a new approach to the delivery of a variety of therapeutics.

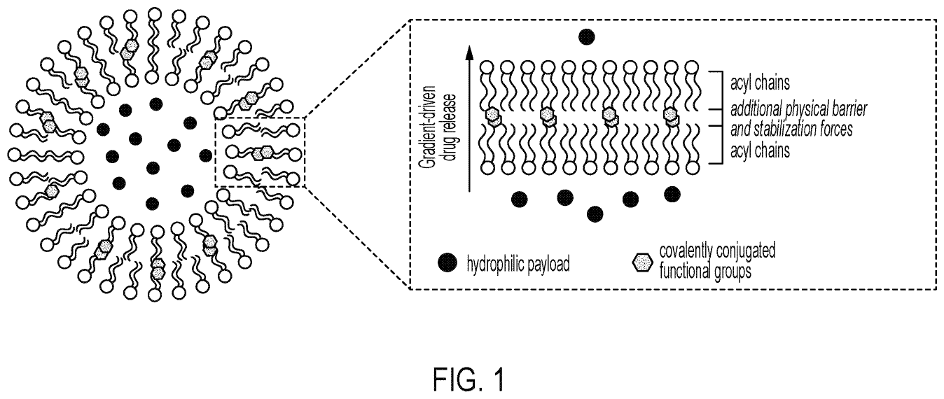

Taking inspiration from the physiochemistry of naturally-occurring lipid bilayers and the passive diffusion of molecules across cell membranes, it is believed that additional stabilization forces that can tighten the packing of lipid bilayers may help retain entrapped molecules and improve drug loading. The incorporation of aromatic groups into lipid bilayers may stabilize the liposomes via p-p stacking interactions. (FIG. 1) Aromatic rings are attracted to one another by a combination of dispersion forces and dipole-induced interactions, known as π-π stacking interactions¹⁷. π-π stacking interactions are considered a special type of van der Waals forces¹⁸. As with hydrophobic interactions, the role of π-π stacking interactions has been explored in the fabrication and stabilization of supramolecular structures¹⁹, including drug delivery systems²⁰.

### Synthesis of Acyl Chain-Modified Phospholipids.

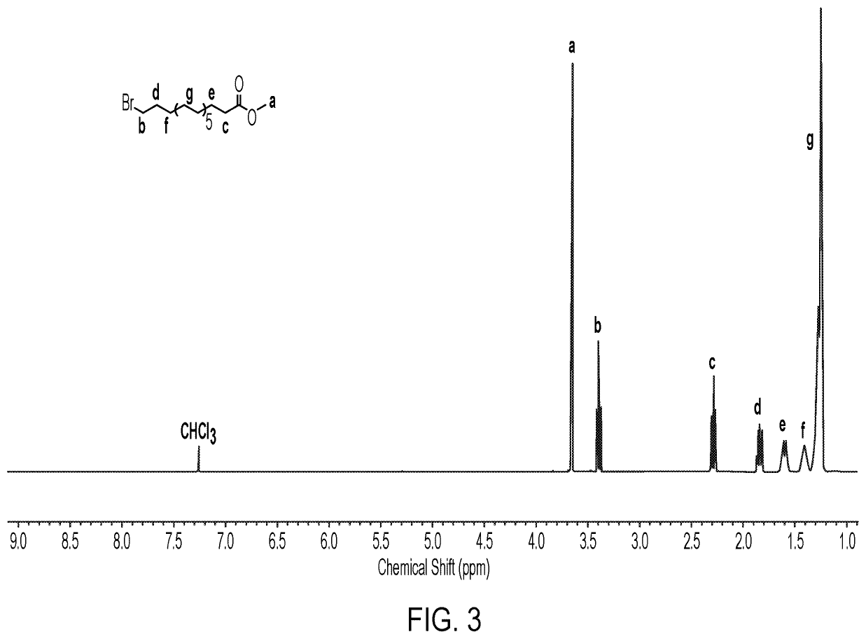

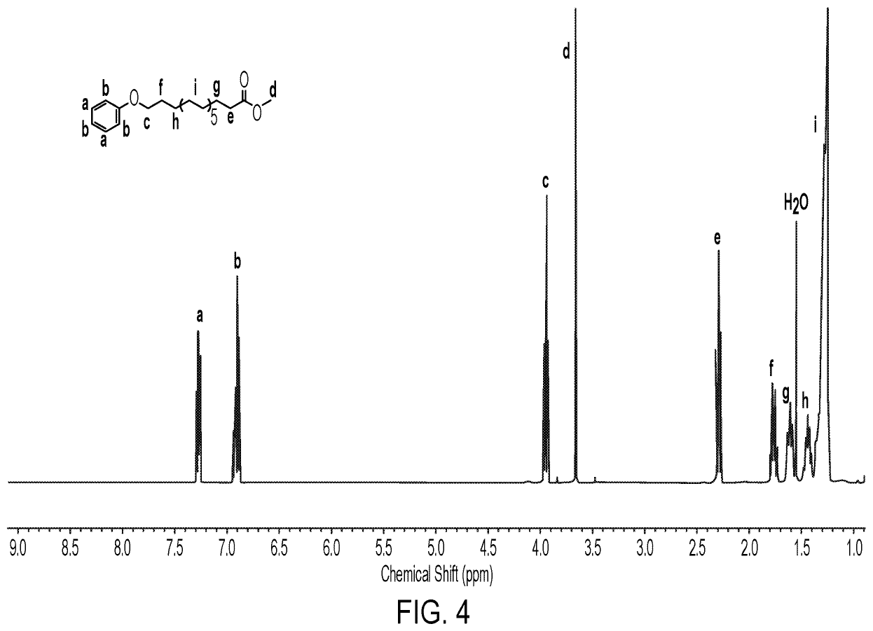

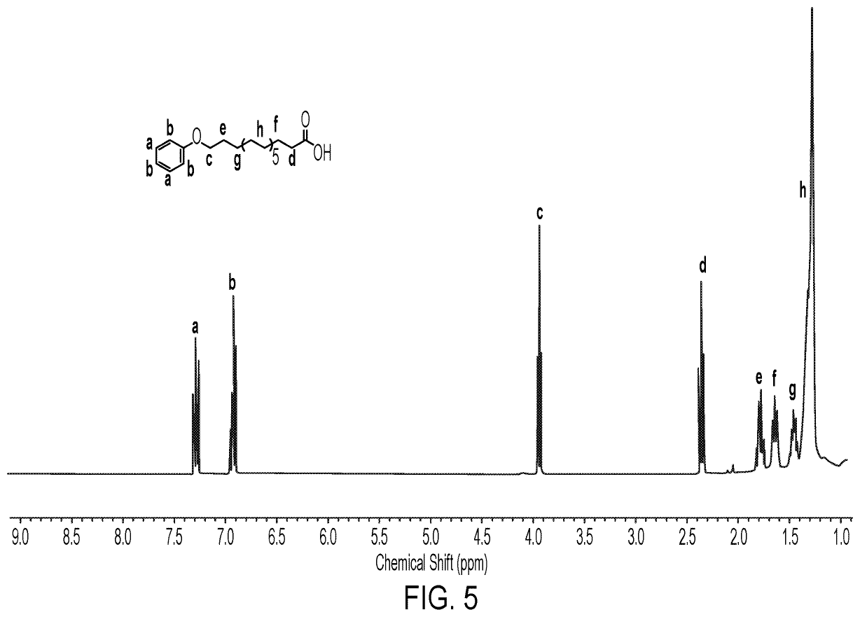

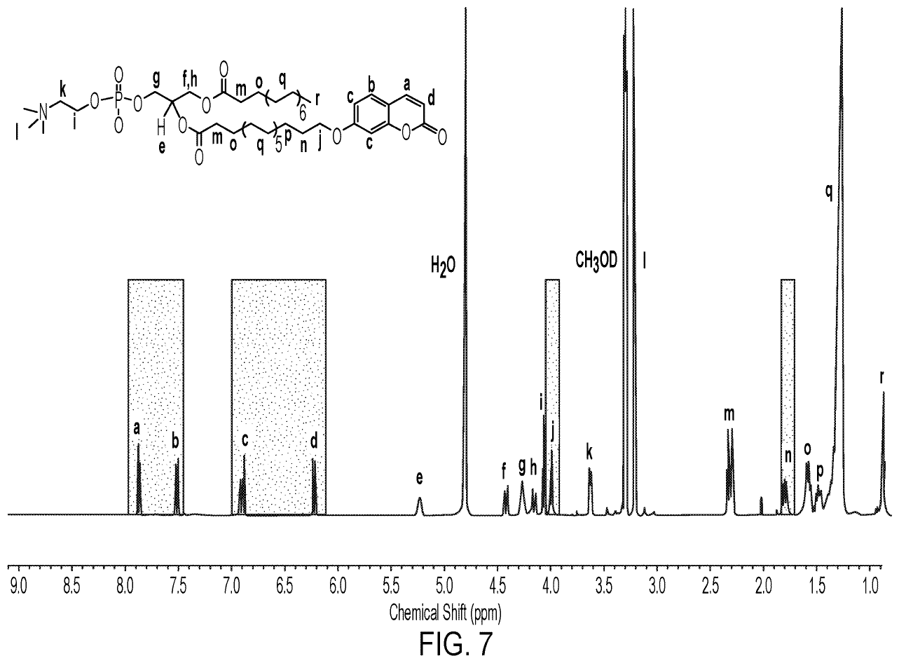

A strategy to conjugate aromatic groups onto the acyl chains of phospholipids was developed (FIG. 2). The process began with the synthesis of phenoxy-conjugated phospholipids because the benzene ring was the smallest aromatic group that could enable n-n stacking interactions²¹. The phenoxy groups were selected because their ether bond was much less polar than amide and ester bonds, minimizing the disruption of the hydrophobic network within lipid bilayers²². Methyl 16-bromohexadecanoate, phenol, and potassium carbonate were mixed in anhydrous acetonitrile at 60° C. overnight to create methyl 16-phenoxyhexadecanoate. Deprotection of the carboxylic acid yielded 16-phenoxy-palmitic acid, which then reacted with 1-palmitoyl-2-hydroxy-sn-glycero-3-phosphocholine (lysoPC) via a coupling reaction to create phenoxy-conjugated DPPC (Ph-DPPC). In the ¹H-NMR spectrum of Ph-DPPC, the representative signals of aromatic rings at 7.2 and 6.9 ppm clearly demonstrated the successful conjugation of a phenoxy group to the acyl chains of the phospholipid (Ph-DPPC) (FIGS. 3-7), which was also confirmed by LC-MS (FIG. 8). Coumarin conjugated DPPC (CM-DPPC) was synthesized following similar procedures. The coupling of lysoPC and functionalized fatty acids represents a general strategy for the synthesis of acyl chain-modified phospholipids.

The yield of the above coupling reaction was extremely low with commonly used coupling reagents such as N,N′-dicyclohexylcarbodiimide (DCC), N,N′-diisopropylcarbodiimide (DIC), and 1-ethyl-3-(3-dimethylaminopropyl) carbodiimide (EDC), probably due to the poor solubility of lysoPC in organic solvents like chloroform. After screening multiple coupling reagents, 2,6-dichlorobezoyl chloride and 1-methylimidazole were used in the synthesis due to their high potency in activating fatty acid, which lead to rapid reaction rates and over 75% yield²³.

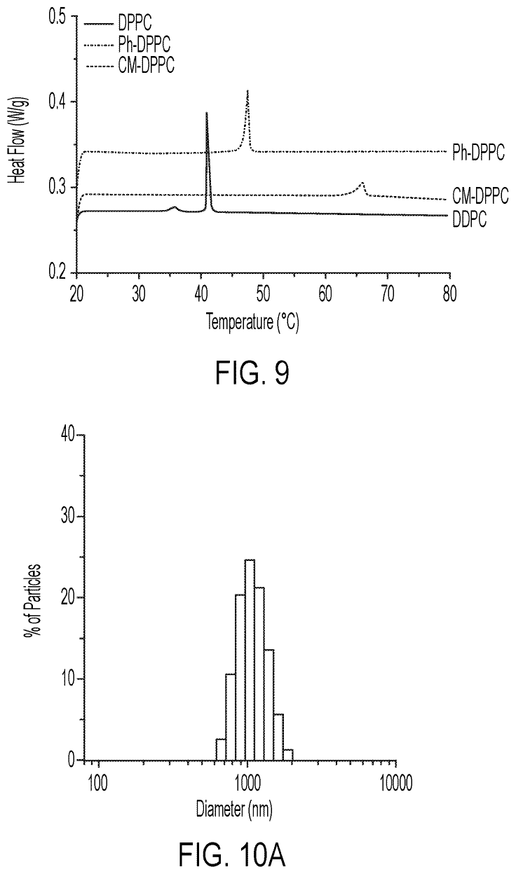

The phase transition temperatures (Tm) of Ph-DPPC and CM-DPPC were 47.4 and 66.1° C., respectively (FIG. 9). Preparation of liposomes containing CM-DPPC required high temperatures due to the high Tm, which may limit their use as carriers for temperature-sensitive cargos like proteins and nucleic acids. Therefore, the focus was on liposomes incorporating Ph-DPPC for drug encapsulation and on release investigations.

### Preparation and Characterization of Aromatized Liposomes.

Aromatized liposomes were prepared following a thin-film hydration method²⁴. In a typical procedure, a dried lipid thin film containing Ph-DPPC, 1,2-dioleoyl-sn-glycero-3-phosphocholine (DOPC), 1,2-distearoyl-sn-glycero-3-phosphatidylglycerol (DSPG), and cholesterol in a 3:3:2:3 molar ratio was hydrated with phosphate buffered saline (PBS). Both phosphocholine (PC) and phosphoglycerol (PG) are needed to maintain the physical stability of liposomes. DOPC is used in these formulations to facilitate membrane fusion and fine tune membrane permeability. DOPC was used for Exparel and Depodur, two clinically approved liposome products for pain relief. DSPG was used because it was found that negatively charged DSPG can enhance the encapsulation of positively charged TTX. DPPC, DSPG and cholesterol (Chol) were used for sciatic nerve injection, showing no nerve damage.

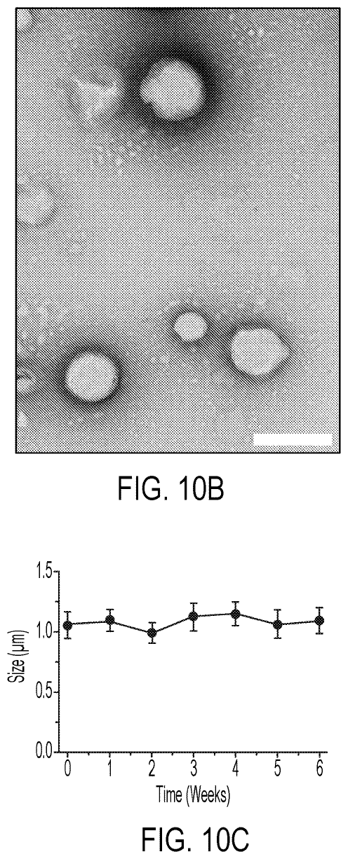

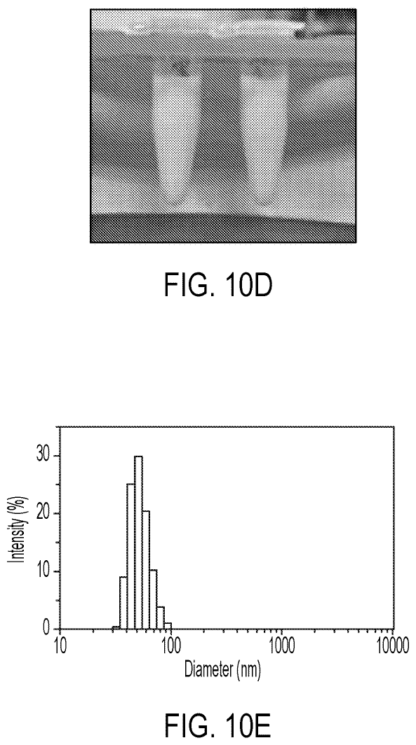

Mechanical agitation was applied during the hydration to facilitate membrane fusion and formation of multilamellar vesicles¹⁰. Combination of Ph-DPPC and natural phospholipids afford aromatized liposomes similar to conventional liposomes as confirmed by dynamic light scattering (DLS) and transmission electron microscopy (TEM) (FIGS. 10A-10B). The liposomes displayed similar size and did not aggregate after storage at 4° C. for several weeks, indicating good stability (FIGS. 10C, 10D). Nanoscale aromatized liposomes can be produced after extrusion through polycarbonate filters (FIG. 10E), which is favorable for systemic administration.

The hydrophilic small molecule fluorophore sulforhodamine B (SRho, log P=−0.53, FIG. 11A) was loaded into liposomes by hydrating the lipid cake with a 10 mg/mL SRho solution in PBS. The loading of SRho in aromatized liposomes (SRho@Lipo-Ph) was 19% greater than in unmodified liposomes (FIG. 11B), showing slightly increased drug loading efficiency as a result of aromatization. The release kinetics of SRho was evaluated in vitro. Free (unencapsulated) dye was released within 6 hours. In SRho@Lipo, more than 12% SRho was released in 24 h. In comparison, less than 6.0% and 5.5% of SRho was released in 24 h from SRho@Lipo-Ph and SRho@Lipo-CM (FIG. 11C). Compared to Lipo-Ph, Lipo-CM did not considerably increase the drug loading or reduce the cumulative drug release in first 24 h. Viscosity is an important factor for injectability. The viscosity of different formulations was characterized at a range of angular frequencies. The aqueous solution of Lipo-CM was notably more viscous than Lipo-Ph (FIG. 11D). Lipo-Ph was chosen for subsequent studies.

The acyl chain of phospholipids was modified with dibenzocyclooctyne (DBCO) (DBCO-DPPC, FIG. 11E), a group with three rings including two aromatic rings. The loading of SRho in DBCO-modified liposomes was 2.3±0.3%, comparable to that in unmodified liposomes (FIG. 11F). 21% of SRho was released from SRho@Lipo-DBCO in 24 h, which was faster than from SRho@Lipo (FIG. 11G). The bulky DBCO group may interfere with the packing of hydrocarbon chains, making the liposome leakier.

Since the covalently conjugated phenyl group slowed the release of payload, the extent to which physical (non-covalent) encapsulation of phenol into lipid bilayers would stabilize liposomes was studied. To compare the effects of covalent conjugation and physical encapsulation, SRho was co-encapsulated with 1 mM phenol into liposomes (Ph+SRho@Lipo) (FIG. 11H). Ph+SRho@Lipo released 7.1% of SRho in 24 h, 42% less than from unmodified liposomes, but 20% more than from covalently aromatized liposomes.

Phenol is intrinsically toxic, as confirmed by cytotoxicity data (FIG. 11I-J). Therefore, in order to be able to assess the effect of non-covalently incorporated aromatic groups in vivo, SRho was co-encapsulated with 1 mM indocyanine green (ICG), an FDA approved fluorophore containing four aromatic rings. SRho release from ICG+SRho@Lipo was less rapid than from unmodified liposomes, but more than from covalently aromatized liposomes (FIG. 11H). Since there was no statistically significant difference between the effect of Ph and ICG on drug release, the latter was used in downstream experiments.

### Effect of Liposome Aromatization on Different Payloads

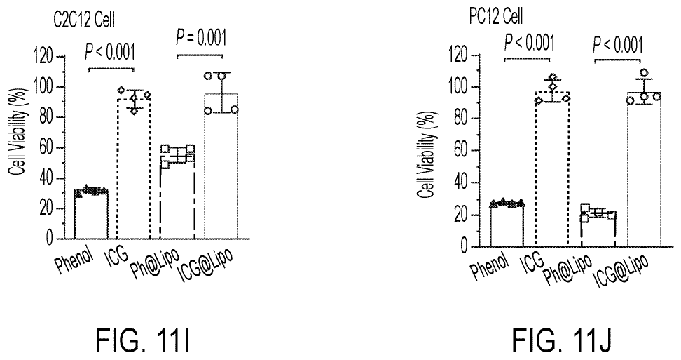

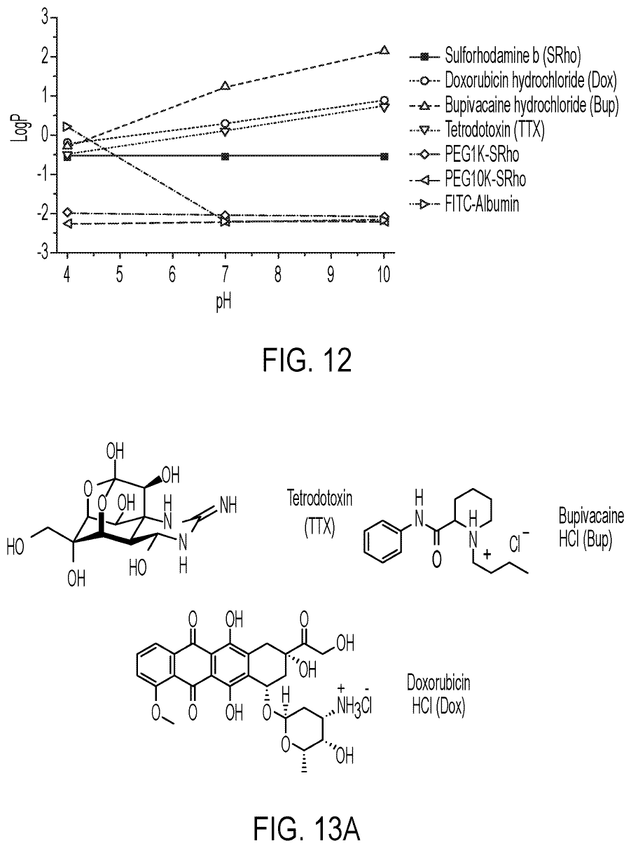

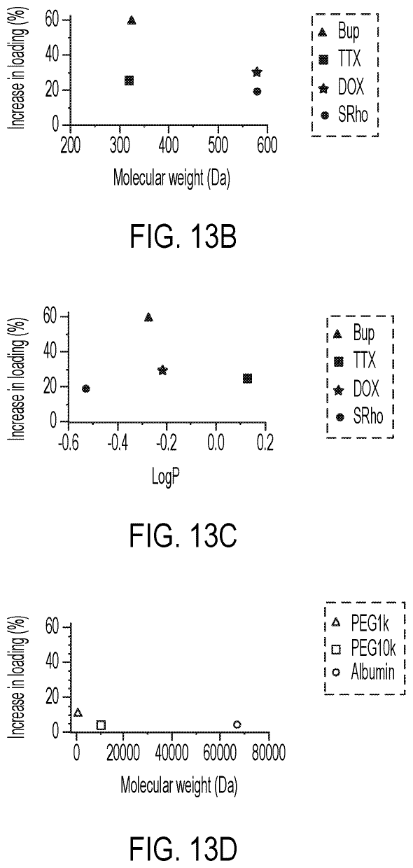

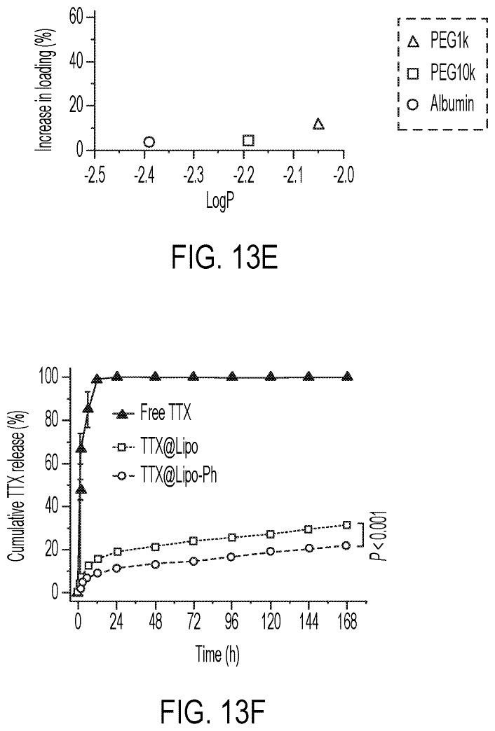

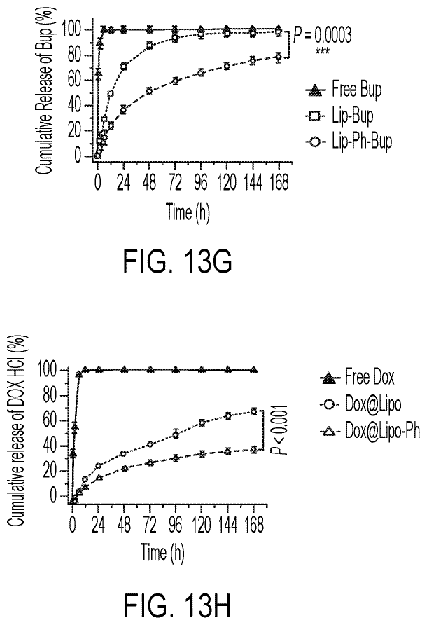

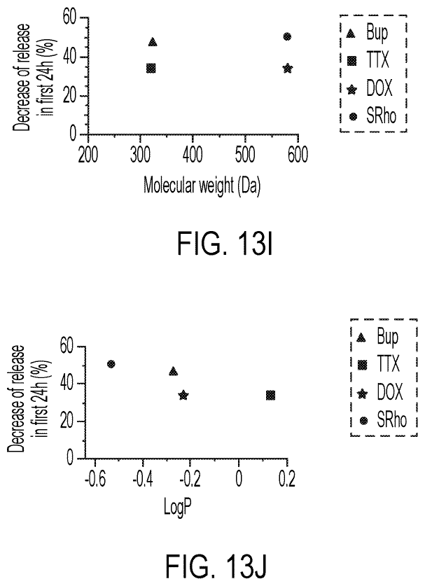

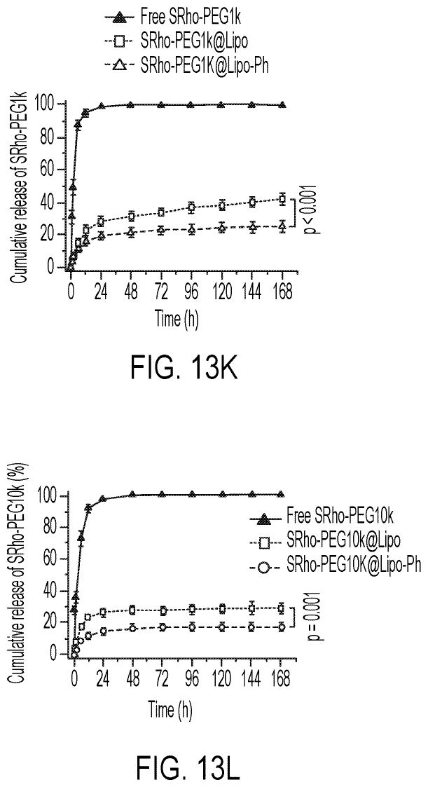

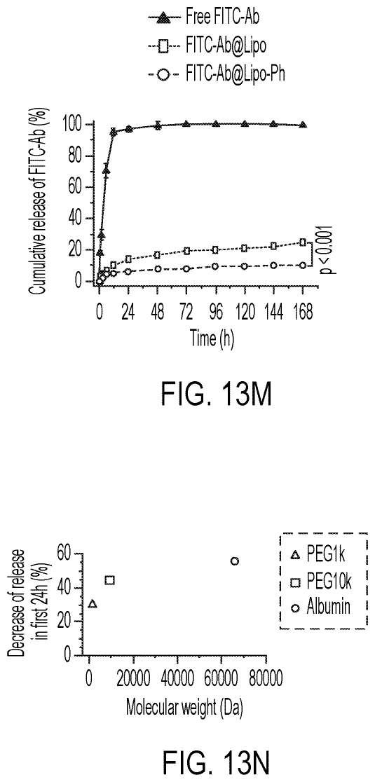

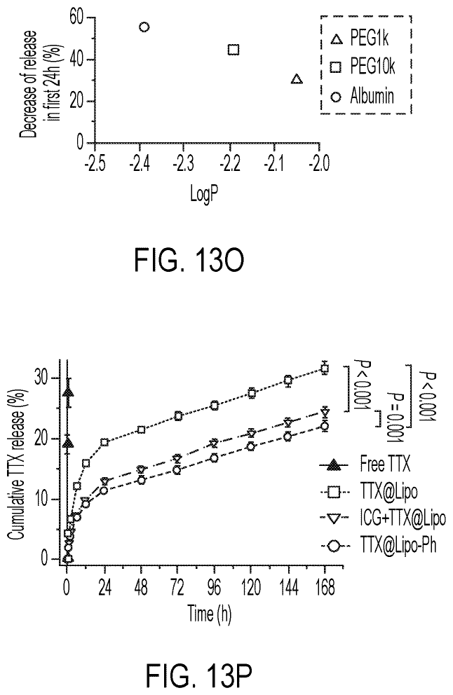

The effect of liposome aromatization to include payload molecules with different characteristics such as hydrophilicity and molecular weight was studied. The hydrophilicity of different payloads was measured by their octanol-water partition coefficients (FIG. 12). In addition to SRho described above, the following small molecule compounds were encapsulated (FIG. 13A), using the same encapsulation and analytical techniques: tetrodotoxin (TTX, log P=0.13), a hydrophilic ultrapotent local anesthetic; bupivacaine hydrochloride (Bup, log P=−0.27), an amphiphilic amino-amide local anesthetic in current clinical use; doxorubicin hydrochloride (Dox, log P=−0.22), a chemotherapeutic drug that has been used clinically in liposomes to treat cancer.

Loading of small molecule compounds was increased by 19-60% by aromatization of liposomes (Table 1). Neither molecular weight (R²=0.32) nor hydrophilicity (R²=0.00) correlated with the degree of increase in drug loading (FIG. 13B-E). Release in the first 24 h was reduced by 30-60% by aromatization of liposomes (FIGS. 13F-H, Table 2). Although molecular weight (FIG. 13I; R²=0.01) did not correlate strongly with the degree of reduction, hydrophilicity showed some correlation with the degree of reduction (FIG. 13J; R²=0.64).

TABLE 1
Effect of aromatization on the loading of different payloads.

| | Drug loading | | | |
|---|---|---|---|---|
| | | | Increase | |
| Payload | Lipo | Lipo-Ph | in loading | P values |
| SRho | 2.6 ± 0.3% | 3.1 ± 0.2% | 19.2% | P = 0.009 |
| TTX | 0.11 ± 0.01% | 0.13 ± 0.01% | 25.5% | P = 0.004 |
| Bup | 3.5 ± 0.7% | 5.6 ± 0.8% | 60.0% | P = 0.006 |
| Dox | 0.8 ± 0.1 | 1.1 ± 0.1% | 30.4% | P = 0.004 |
| SRho-PEG1k | 0.78 ± 0.04 | 0.87 ± 0.02 | 11.5% | P = 0.011 |
| SRho-PEG10k | 1.64 ± 0.13 | 1.71 ± 0.02 | 4.3% | P > 0.05 |
| FITC-Ab | 3.63 ± 0.20 | 3.78 ± 0.16 | 4.0% | P > 0.05 |

TABLE 2
Effect of aromatization on the release of different payloads.

| | Cumulative release in first 24 h | | | |
|---|---|---|---|---|
| | | | Decrease | |
| Payload | Lipo | Lipo-Ph | in release | P values |
| SRho | 12.2 ± 0.5% | 5.9 ± 0.7% | 51.6% | P < 0.001 |
| TTX | 17.1 ± 0.3% | 11.3 ± 0.4% | 34.0% | P < 0.001 |
| Bup | 70.7 ± 1.9% | 37.0 ± 2.7% | 47.7% | P < 0.001 |
| Dox | 26.8 ± 1.1% | 17.6 ± 1.0% | 34.3% | P < 0.001 |
| SRho-PEG1k | 28.2 ± 2.5% | 19.6 ± 2.0% | 30.5% | P = 0.002 |
| SRho-PEG10k | 26.2 ± 2.3% | 14.5 ± 2.1% | 44.6% | P < 0.001 |
| FITC-Ab | 14.2 ± 1.2% | 6.3 ± 0.8% | 55.6% | P < 0.001 |

Similar loading and release experiments were done with liposomes loaded with the macromolecules rhodamine-conjugated polyethylene glycol (1 kDa and 10 kDa, Log P=−2.05 and −2.19, respectively) and fluorescein isothiocyanate-conjugated albumin (Log P=−2.39, FIG. 12). Although aromatization had a modest (or no) effect on drug loading of macromolecules (Table 1), aromatization decreased the release of macromolecules in the first 24 h from liposomes (FIG. 13K-M). The effect of liposomal aromatization on drug release increased with increasing molecular weight (FIG. 13N; R²=0.81) and increasing hydrophilicity (FIG. 13O; R²=0.97).

These data showed that aromatized liposomes had benefits over conventional liposomes for delivery of a wide range of small and large molecules.

In anticipation of in vivo experimentation, TTX was also co-encapsulated in unmodified liposomes with ICG (ICG+TTX@Lipo). 13% of TTX was released from ICG+TTX@Lipo within 24 h, which was less rapid than from unmodified liposomes, but more than from aromatized liposomes in the same period (FIG. 13P).

Tetrodotoxin (TTX) was used to assess the effect of aromatized liposomes on sustained release of lower molecular weight drugs. The high potency and minimal cardiac and tissue toxicity of TTX made it an appealing local anesthetic²⁵. However, systemic toxicity can be dose-limiting²⁶, even when it is encapsulated²⁷. The extremely high hydrophilicity (Log P=−6.2), low molecular weight (319), and narrow therapeutic window make the encapsulation and controlled delivery of TTX challenging²⁸.

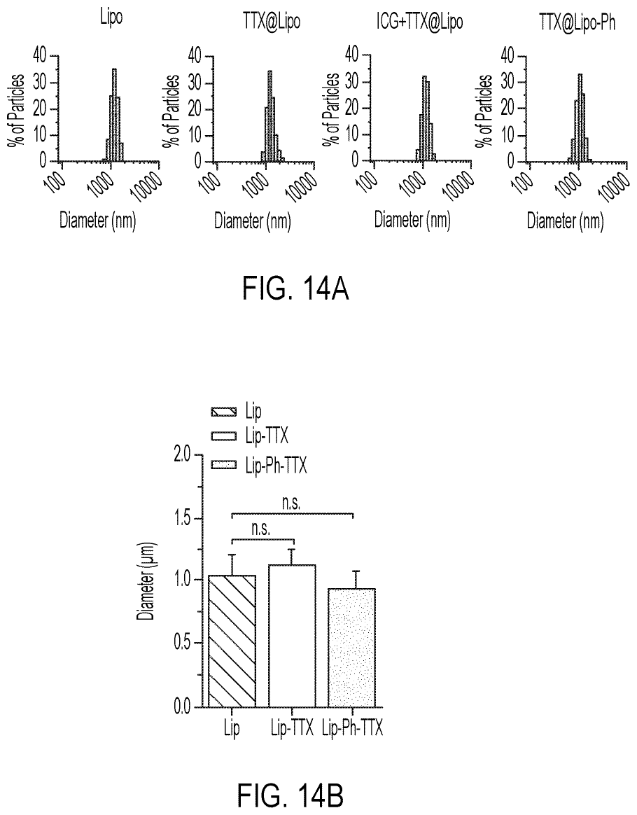

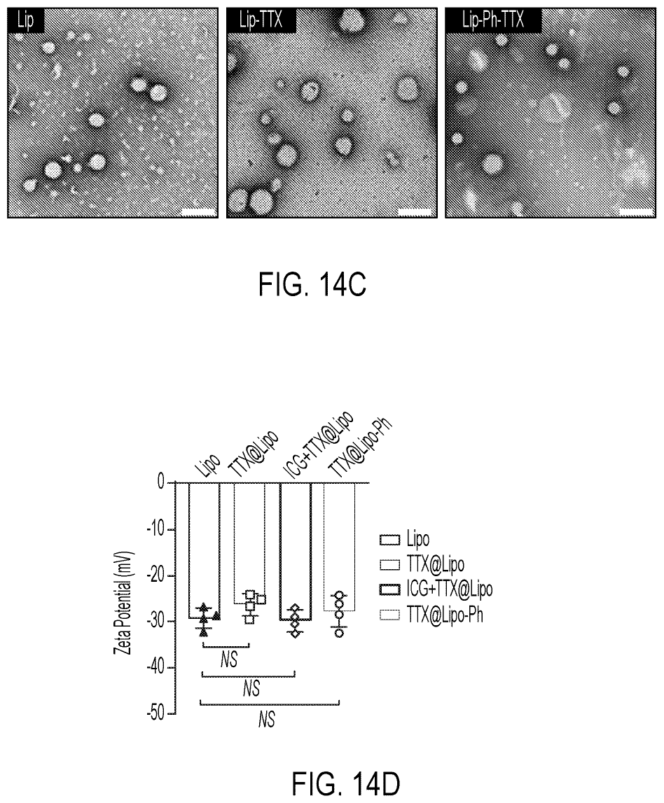

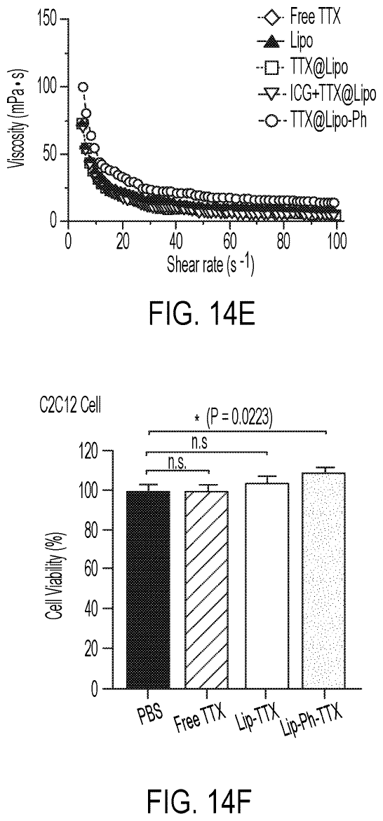

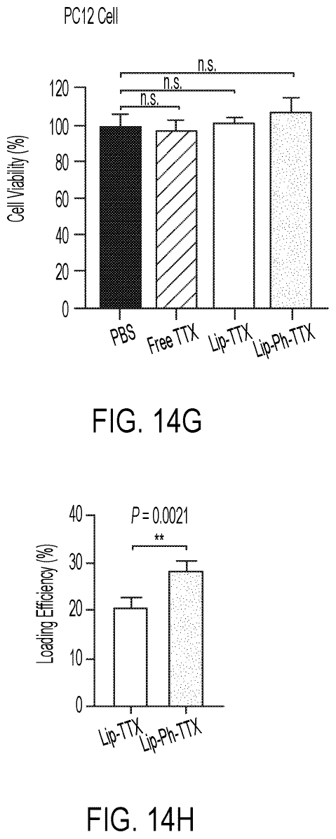

Liposomes encapsulating TTX were prepared by the same hydration method. Liposomes fabricated with or without Ph-DPPC showed similar median number-weighted diameters around 1 m (FIGS. 14A-14B; Table 3). TEM confirmed the formation of spherical vesicles (FIG. 14C). All liposomes showed similar negative zeta potentials around −30 mV, indicating that the incorporation of aromatized phospholipids or encapsulation of TTX did not change their surface charge (FIG. 14D). Incorporation of aromatic groups within lipid bilayers did not significantly alter the size, zeta potential, or polydispersity of liposomes. All liposomal solutions showed very low viscosity of less than 10 mPa·s (FIG. 14E), which was not significantly higher than that of PBS (1.9 mPa·s), suggesting easy injectability.

TABLE 3
Characterization of liposomal formulations.

| | Diameter | Zeta Potential | Polydispersity |
|---|---|---|---|
| Formulations | (mm) | (mV) | Index |
| Lipo | 1.21 ± 0.19 | −29.2 ± 2.3 | 0.192 ± 0.061 |
| TTX@Lipo | 1.26 ± 0.14 | −26.5 ± 1.2 | 0.122 ± 0.037 |
| ICG + TTX@Lipo | 1.38 ± 0.31 | −29.9 ± 2.5 | 0.125 ± 0.070 |
| TTX@Lipo-Ph | 1.14 ± 0.17 | −27.7 ± 3.5 | 0.159 ± 0.046 |

Data are means ± s.d., n = 4.

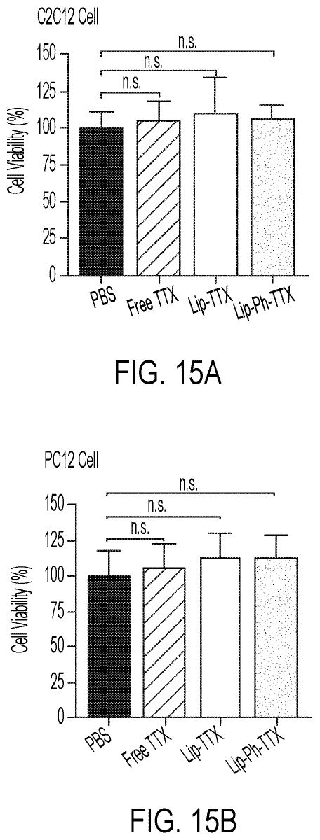

The cytotoxicity of aromatized liposomes was evaluated in myoblast C2C12²⁹ and pheochromocytoma PC12³⁰ cell lines to assess potential cytotoxicity to muscle and nerve cells, respectively. Cell viability, assessed by MTS assays, was very high in all tested groups (FIGS. 14F-14G and FIGS. 15A-15B), indicating that incorporation of synthetic Ph-DPPC did not compromise the biocompatibility of the liposomes.

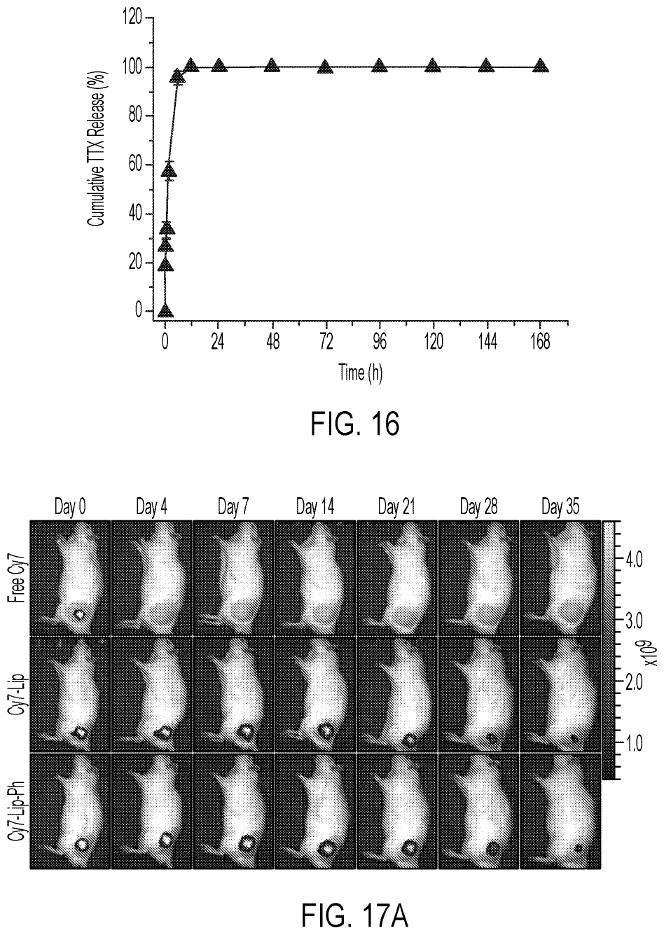

Aromatized liposomes encapsulating TTX (Lip-Ph-TTX) showed increased TTX loading compared to unmodified ones (Lip-TTX) (FIG. 14H). Release of TTX from liposomes was quantified by enzyme-linked immunosorbent assay (ELISA). More than 95% TTX was released after 8 hours in the experimental set-up (FIG. 16). Physical-Encapsulation into Lip-TTX enabled sustained release of TTX over 168 hours. However, rapid burst release was still observed, where nearly 20% of encapsulated TTX was released in 24 hours (FIG. 13F). Lip-Ph-TTX significantly reduced the burst release of TTX compared to Lip-TTX.

### Localization and Retention of Liposomal Formulations.

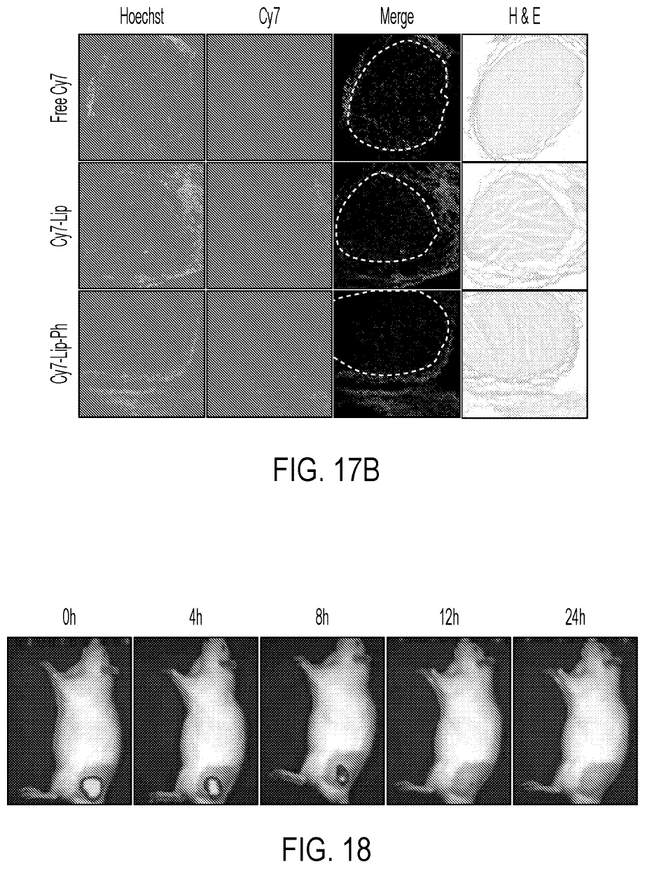

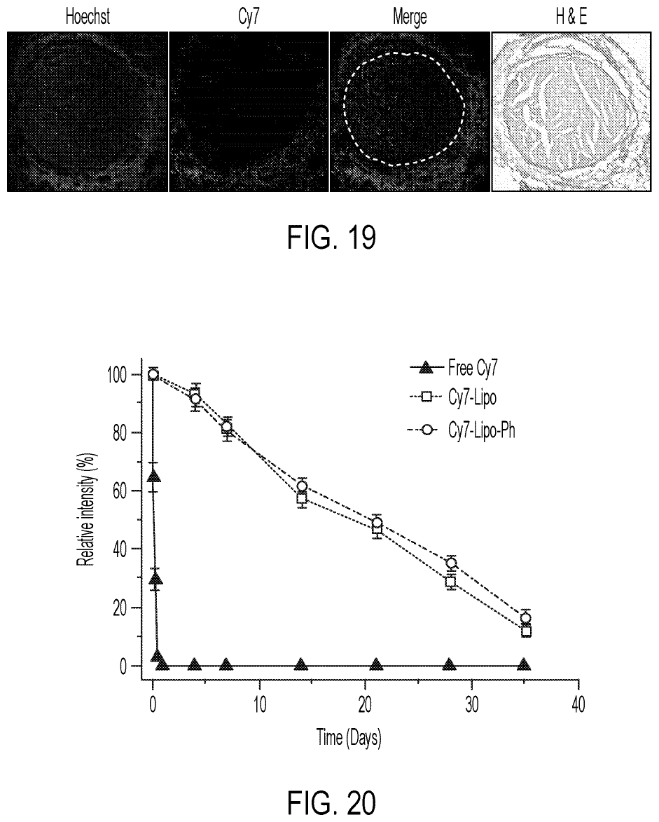

To assess whether lipid bilayer engineering affected the local retention of liposomes, liposomes were covalently conjugated with Cy7 and injected at the sciatic nerve site. Fluorescence images of rats were then taken at different time points using an in vivo imaging system (IVIS) (FIG. 17A). While free Cy7 (fluorophore is not covalently conjugated to any compound or phospholipid) rapidly diffused away from the injection site within hours (FIG. 17A and FIG. 18), both unmodified liposomes (Cy7-Lip) and aromatized liposomes (Cy7-Lip-Ph) remained there for several weeks, consistent with previous studies³². Fluorescent confocal microscopy confirmed the localization of Cy7-conjugated liposomes as well as free dyes in the connective tissue between muscles and nerves (FIGS. 17B, 19, and 20). These findings suggested that differences in anesthetic effect between formulations would be due to differences in drug release, not tissue retention. “Cy7-Lip-Ph” refers to Cy7

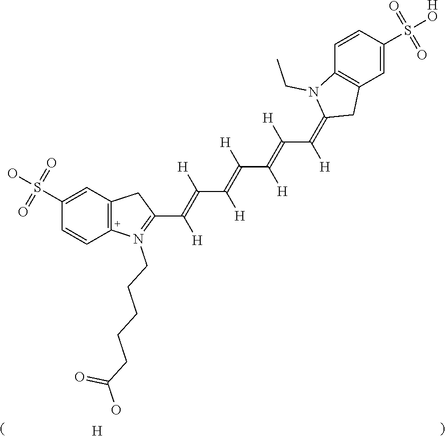

that is covalently conjugated to the phospholipid.

### In Vivo Therapeutic Efficacy and Toxicity.

To assess whether the sustained release of drug with aromatized liposomes could translate into improved duration of effect and reduced toxicity in vivo, the liposomes were used in a rat model of peripheral nerve block. Rats were injected at the left sciatic nerve with 300 μL of different TTX formulations (FIG. 21A). Neurobehavioral testing based on a modified hot-plate test was applied in both hind paws to assess the duration of functional deficits²⁴. Local anesthesia was assessed by the length of time a rat would leave its hind paw on a hotplate (thermal latency). The duration of deficits on the injected (left) side reflected the duration of nerve block, whereas deficits on the contralateral un-injected (right) side indicated numbness from systemic distribution of TTX.

Rats receiving sciatic nerve injection of free TTX showed dose-dependent nerve blockade, where 2 μg of TTX in PBS buffer showed no detectable block while 4 μg of free TTX in PBS buffer enabled a median duration of sensory block of 2.5±0.6 hours (FIGS. 21B-21D). However, 4 μg of free TTX induced systemic toxicity, as reflected by a sensory deficit lasting 2.1±0.5 hours in the contralateral leg (FIG. 21E-21F). Injection of 5 μg of free TTX was uniformly fatal (FIG. 21G).

Nerve block duration and safety were improved by encapsulation of TTX in liposomes (Lip-TTX) (FIG. 21B). The actual dose in each formulation varied slightly due to differences in TTX loading efficiency (Table 4). The onset of nerve blockade occurred 10-15 minutes after injection of Lip-TTX, and resulted in a duration of 19.9±4.4 hours (FIG. 21C). In addition, Lip-TTX improved the safety of TTX as evidenced by the decrease in contralateral deficits (FIG. 21E). The contralateral block occurred in 62.5% of animals (FIG. 21F). The peak latency in the contralateral leg was 7.5±1.0 seconds, shorter than 12 seconds (maximum thermal latency, rats were removed manually after 12 seconds to prevent potential thermal injury) with free TTX. 3 out of 4 rats died when TTX@Lipo containing 24.8 μg TTX was injected. Higher loadings of TTX (29.6 μg) were fatal. Contralateral deficits were not detectable 6 hours after injection, indicating that sustained release of TTX from liposomes significantly decreased systemic toxicity.

TABLE 4
Comparison of loading efficiency and loading
in different TTX formulations.

| | | Loading | Drug |
|---|---|---|---|
| Formulations | Payload | Efficiency (%) | Loading (%) |
| Lipo | — | — | — |
| TTX@Lipo | TTX | 22.6 ± 1.9 | 0.106 ± 0.009 |
| ICG + TTX@Lipo | ICG | 84.3 ± 3.8 | 0.524 ± 0.024 |
| | TTX | 27.1 ± 1.7 | 0.127 ± 0.008 |
| TTX@Lipo-Ph | TTX | 28.5 ± 2.0 | 0.133 ± 0.009 |

Data are means ± s.d., n = 4.

Aromatized liposomes (Lip-Ph-TTX) further improved the therapeutic duration and safety of TTX (FIG. 21B). The onset of nerve blockade occurred 20-30 minutes after the injection of Lip-Ph-TTX, and produced 100% successful block in all animals. Nerve block from TTX@Lipo-Ph containing 25.3 μg TTX (FIG. 21D) lasted 36.9±4.6 h (˜2-fold the duration obtained with 20.4 μg TTX in TTX@Lipo), there was no contralateral block (FIG. 21F), and there were no animal deaths (FIG. 21G). Lip-Ph-TTX containing 32.1 μg of TTX achieved a median duration of sensory block to 57.1±11.6 h, 23-fold greater that from 4 g free TTX and 2.9 times longer than that from Lip-TTX (FIG. 21C). This dose was higher than a dose that was uniformly fatal in animals receiving TTX@Lipo (29.6 μg TTX). TTX@Lipo-Ph containing 37.5 μg TTX enabled nerve block lasting 64.5±8.1 h. Increasing the TTX loading to 44.2 μg further increased the duration of block to 70.7±9.8 h (Table 5). Even at that loading, there were no animal deaths, and contralateral block only occurred in 50% of animals (lower than the percentage with 20.4 μg TTX in TTX@Lipo). The peak latency in the contralateral leg was further decreased to 4.1±0.4 seconds and the contralateral deficits could not be detected after 4 hours (FIG. 21E). These data were consistent with the slower release kinetics of TTX@Lipo-Ph and demonstrated that liposome aromatization resulted in prolonged block and reduced toxicity.

TABLE 5
In vivo assessment of TTX formulations.

| | | Duration of | Contralateral |
|---|---|---|---|
| | TTX dose | nerve block | peak latency |
| Formulation | (μg) | (h) | (s) |
| Free TTX | 4.0 | 2.5 ± 0.6 | 12 ± 0.0 |
| TTX@Lipo | 20.4 | 19.9 ± 4.4 | 7.5 ± 1.0 |
| ICG + TTX@Lipo | 24.4 | 26.9 ± 5.5 | 6.7 ± 0.9 |
| TTX@Lipo-Ph | 25.3 | 36.9 ± 4.6 | 3.3 ± 0.4 |
| | 32.1 | 57.1 ± 11.6 | 4.2 ± 0.5 |
| | 37.5 | 64.5 ± 8.1 | 5.0 ± 0.4 |
| | 44.2 | 70.7 ± 9.8 | 7.5 ± 1.2 |

Data are means ± s.d., n = 4.

Stabilization of liposomes by n-n stacking could also be achieved by physical encapsulation of hydrophobic aromatic molecules into lipid bilayers. ICG, which contains four aromatic rings, could stabilize lipid bilayers via additional hydrophobic interactions with fatty acids and π-π stacking interactions with each other³³. To compare the effect of covalent conjugation and physical encapsulation, hydrophobic indocyanine green (ICG, log P=6.05)³, an FDA approved fluorophore, was encapsulated into liposomes. Lip-ICG-TTX, in which ICG and TTX were co-encapsulated into unmodified liposomes containing 24.4 μg TTX (structure of tetrodotoxin or TTX which is shown below), achieved sensory nerve block lasting 26.9±5.5 hours (FIGS. 21C-D), 10.8-fold that with 4 μg free TTX, but 2.1 times less than that achieved by Lip-Ph-TTX. Encapsulation of ICG (structure of indocyanine green or ICG which is shown below) also decreased the peak latency for contralateral leg to 6.6±0.9 seconds (FIG. 21E). Contralateral block occurred in 37.5% of animals (FIG. 21F; similar to that from TTX@Lipo) and there were no animal deaths (FIG. 21G). 2 of 4 animals administered 31 μg TTX in ICG+TTX@Lipo died. All animals receiving 38 μg TTX in ICG+TTX@Lipo died.

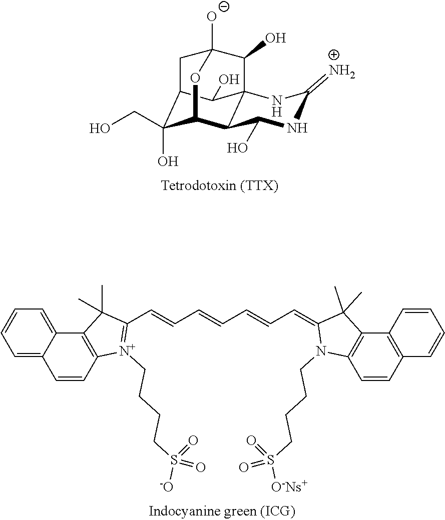

Since the performance of liposomal TTX could be improved by both encapsulation and covalent modification with aromatic compounds, it was assessed whether combining the two approaches within the same liposome formulation (Lip-Ph-ICG-TTX) would further improve the therapeutic duration and mitigate the toxicity of TTX. Lip-Ph-ICG-TTX containing 35.8 μg TTX achieved the most prolonged nerve blockade, 73.5±17.4 hours (FIG. 21C). Moreover, Lip-Ph-ICG-TTX also displayed the least systemic TTX distribution as evidenced by the peak contralateral latency of 3.9±0.4 seconds (FIG. 21E).

Whether the codelivery of encapsulated adjuvant compounds with aromatized liposomes can further enhance the nerve block duration was investigated. Previous studies have combined S1SCBs like TTX with adjuvants with α-adrenergic activity like epinephrine and glucocorticoid receptor agonists like dexamethasone for prolonged nerve blockade. TTX@Lipo-Ph containing 45.1 μg TTX combined with epinephrine (Epi, 3.6 μg) in the injectate (Epi+TTX@Lipo-Ph) and dexamethasone (Dex, 290 μg) in the liposome (Dex@Lipo-Ph+TTX@Lipo-Ph) markedly increased the nerve block duration to 128.8±22.3 h and 186.5±23.8 h respectively (FIG. 21H). No contralateral block was observed in these groups. Prolonged duration local anesthesia (PDLA) lasting weeks is desirable for treating prolonged pain like cancer pain.

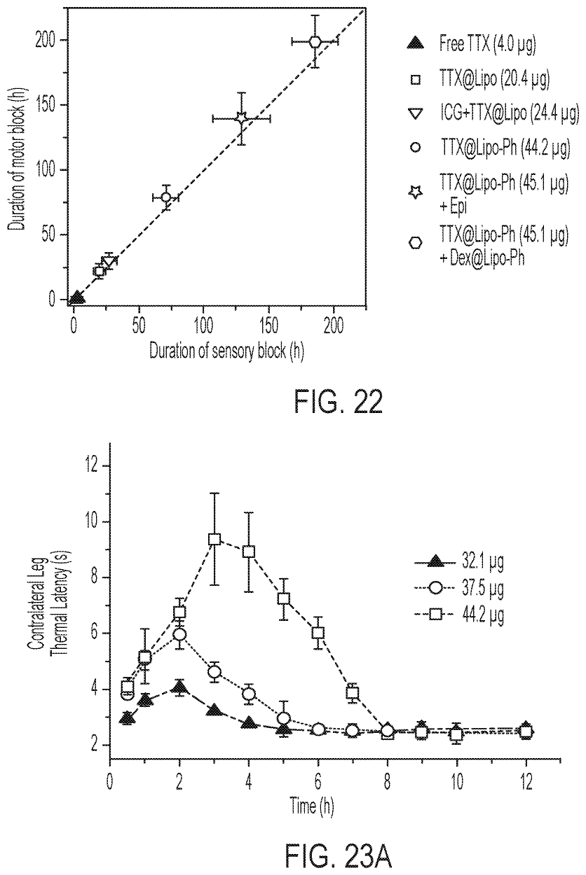

Motor nerve blockade was assessed by a weight-bearing test to determine the motor strength of the rat's hind paw. Aromatized liposomes also significantly prolonged the duration of the motor block (FIG. 22). For free TTX, there was no statistically significant difference between the duration of sensory and motor nerve block. For the liposome formulations, the durations of motor block were 8-13% longer than those of sensory block.

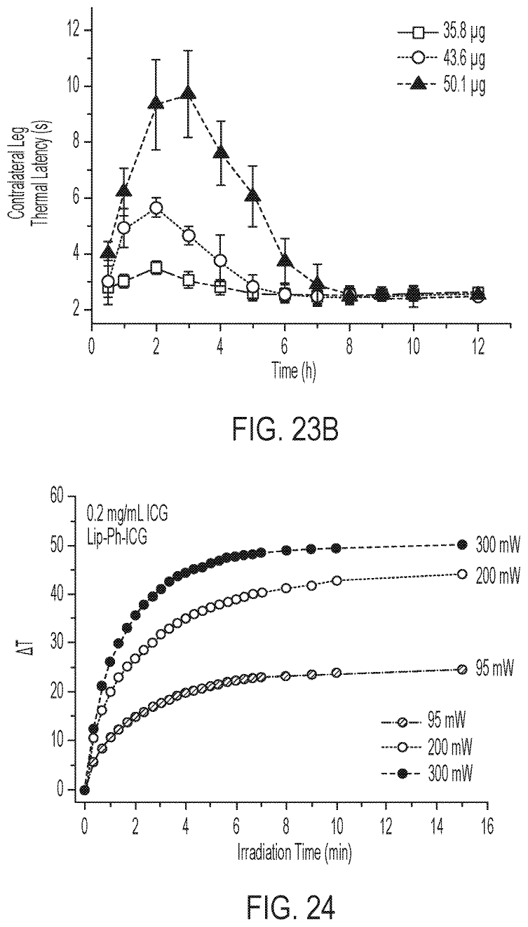

Systemic toxicity limits the dosing of TTX. A low dose (<2 μg) of TTX did not induce detectable nerve block in rats while a high dose (>5 μg) was uniformly lethal (FIG. 21G). With Lip-TTX, death did not occur when the TTX dose was 20 μg (FIG. 21G). Lip-ICG-TTX increased the maximum dose of TTX to 24.4 g. For Lip-Ph-TTX, no animal death was observed even when 45 μg of TTX were administered, and none with 54 μg TTX in Lip-Ph-ICG-TTX. However, increasing the dose of TTX in Lip-Ph-TTX and Lip-Ph-ICG-TTX led to significantly increased contralateral deficits in uninjected legs (FIG. 23), suggesting dose-dependent systemic toxicity.

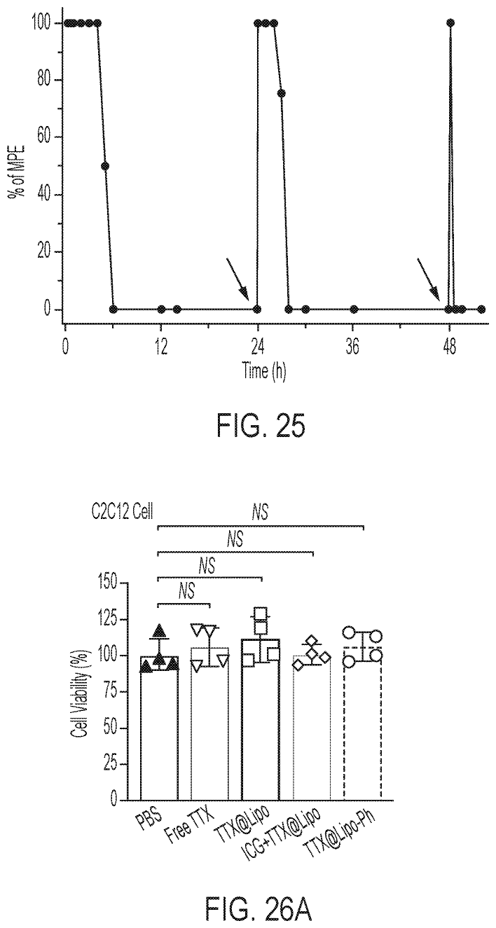

Encapsulation of ICG also enabled triggerable drug release as results of photothermal effect of ICG (FIG. 24) Neurobehavior test showed that TTX-loaded ICG indeed significantly prolonged anesthesia duration and enabled repeatable light-triggered nerve blockade (FIG. 25), indicating incorporation of additional stabilization forces within lipid bilayers have great potential for on-demand pain relief.

### Cytotoxicity and Tissue Reaction

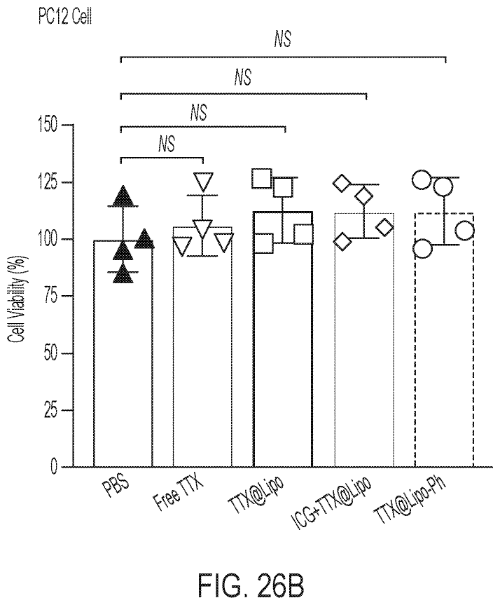

The in vitro cytotoxicity to muscle and nerve of TTX formulations was evaluated in myoblast C2C1229 and pheochromocytoma PC1230 (FIGS. 26A-B) cell lines, that are used to assess myo- and neuro-toxicity respectively. After 48 h of incubation with different TTX formulations (TTX, TTX@Lipo, ICG+TTX@Lipo and TTX@Lipo-Ph), cell viabilities as assessed by MTS assays were similar to that of PBS, indicating that liposomes containing Ph-DPPC did not cause cytotoxicity.

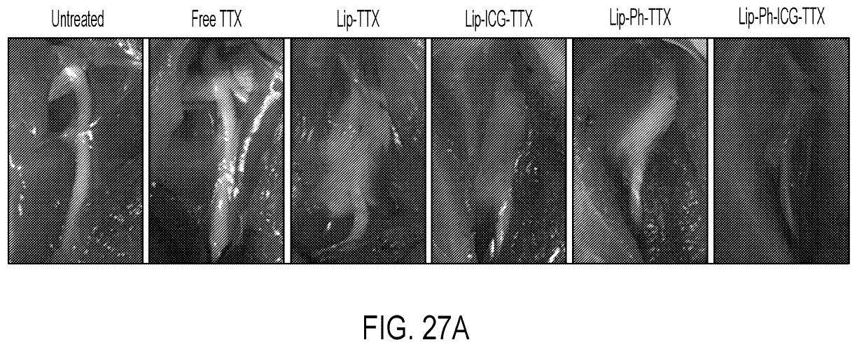

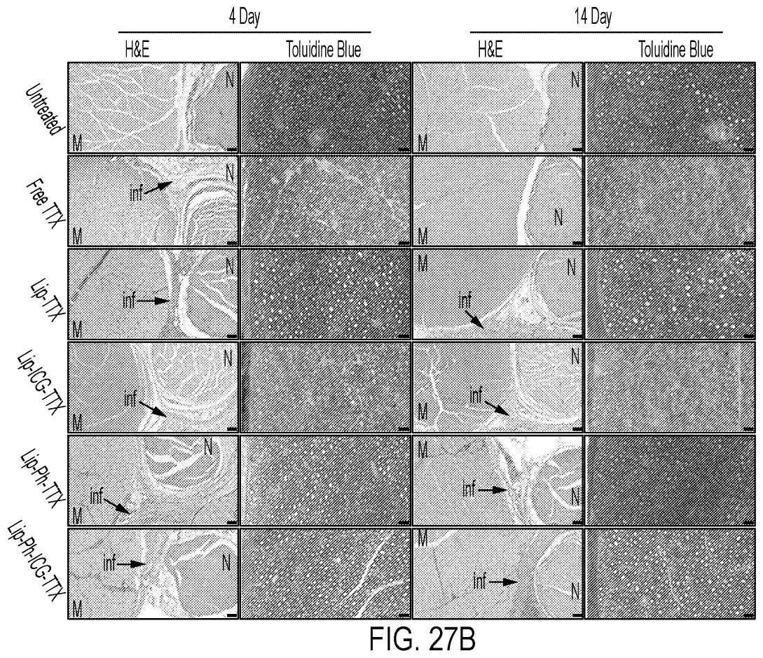

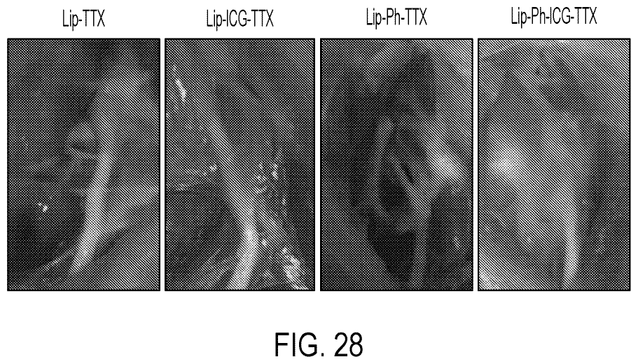

Rats injected with different TTX formulations were euthanized 4 days and 14 days after sciatic nerve injections. At dissection, liposomes could be seen at the sciatic nerve, indicating the accurate injection of TTX-encapsulated liposomes (FIG. 27A). Small amounts of residual liposomes could still be identified at the injection site after 14 days (FIG. 28), consistent with the IVIS results.

Myotoxicity, neurotoxicity, and inflammation are well established side effects of local anesthetics³⁵, although with site 1 sodium channel blockers tissue reaction is due to the vehicle, not the anesthetic. Drug delivery systems themselves are known to cause inflammation that can outlast the duration of nerve block and may enhance local anesthetic myotoxicity.

Animals injected with liposome-TTX formulations were euthanized 4 days or 14 days after injection. The sciatic nerves and surrounding tissues were harvested, sectioned, and stained. Tissue reaction was assessed by hematoxylin and eosin (H&E) staining (FIG. 27B). There was no statistically significant difference between the scores in Lip-TTX, Lip-ICG-TTX, Lip-Ph-TTX, and Lip-Ph-ICG-TTX (Table 6). No myotoxicity in animals in any group 4 days or 14 days after injection (Table 7). Mild to moderate inflammation was seen around the sciatic nerves, and there was no statistically significant difference in myotoxicity scores between these groups and the untreated groups. Inflammation is also commonly found in a broad range of delivery systems, including those for local anesthetics³⁶, and is generally considered to be safe³⁷. The inflammation and myotoxicity scores of the formulations are comparable or better than those of Exparel in the same animal model, suggesting the observed tissue reactions are acceptable for potential clinical use.

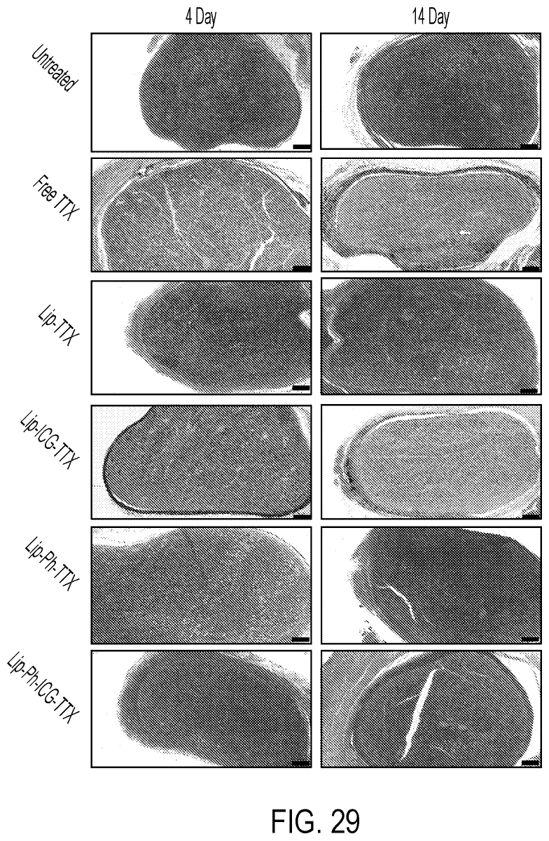

Sciatic nerves were stained with toluidine blue due to the low sensitivity of H&E staining for nerve damage (FIG. 27B and FIG. 29). No neurotoxicity or nerve injury was observed in any group, regardless of the time point. The perineural tissues were normal. Compared to untreated sciatic nerves, no changes were observed in axonal density or myelin structure in any TTX formulations.

TABLE 6
Inflammation 4 and 14 days after injection of formulations.

| Liposomal | Inflammation Scores | | | | |
|---|---|---|---|---|---|
| formulation | Day 4 | | P value | Day 14 | P value |
| Untreated | 0 | (0-0) | — | 0 (0-0) | — |
| Free TTX | 0 | (0-1) | >0.05 | 0 (0-0) | >0.05 |
| TTX@Lipo | 3 | (3-3) | <0.001 | 2 (2-2) | <0.001 |
| ICG + TTX@Lipo | 2 | (2-2) | <0.001 | 1 (0-2) | 0.040 |
| TTX@Lipo-Ph | 2.5 | (2-3) | <0.001 | 2 (1-3) | 0.003 |

Inflammation scores: 0-4. P values result from the comparison of liposomal formulations to the untreated group (Mann Whitney U test). There were no statistically significant differences between liposomal formulations. Data are medians with 25th and 75th percentiles in parentheses; n = 4.

TABLE 7
Myotoxicity 4 and 14 days after injection of formulations.

| Liposomal | Myotoxicity Scores | | | | | |
|---|---|---|---|---|---|---|
| formulation | Day 4 | | P value | Day 14 | | P Value |
| Untreated | 0 | (0-0) | — | 0 | (0-0) | — |
| Free TTX | 0 | (0-0) | >0.05 | 0 | (0-0) | >0.05 |
| TTX@Lipo | 0.5 | (0-4) | >0.05 | 0.5 | (0-1) | >0.05 |
| ICG + TTX@Lipo | 0 | (0-0) | >0.05 | 0 | (0-0) | >0.05 |
| TTX@Lipo-Ph | 0 | (0-0) | >0.05 | 0.5 | (0-4) | >0.05 |

Myotoxicity scores: 0-6. P values result from the comparison of liposomal formulations to the untreated group (Mann Whitney U test). There was no statistically significant difference in myotoxicity scores between different liposome groups and the untreated groups. Data are medians with 25th and 75th percentiles in parentheses from four independent rats.

### Analysis

By targeting the rate-limiting step in the release of encapsulated molecules from liposomes, it was shown here that the aromatized liposomes which incorporating aromatic groups within lipid bilayers for drug loading can sustain drug delivery. A chemical strategy was conceived to conjugate aromatic groups to the acyl chains of phospholipids. This synthetic route can be used for the preparation of a variety of acyl chain-modified phospholipids. Combination of aromatic groups-conjugated phospholipids and natural phospholipids formulates aromatized liposomes with similar size, zeta potential, and polydispersity to conventional liposomes. Release kinetics revealed that aromatized liposomes enabled increased drug loading and decreased release rate of hydrophilic payloads with different water solubility, molecular weight, and charge state. In vivo assessment revealed that aromatized liposomes significantly prolonged the therapeutic duration of encapsulated TTX, a local anesthetic with low molecular weight, high potency and narrow therapeutic window. Lip-Ph-TTX achieved more than 57 hours of continuous nerve block, compared to that of free TTX (2.5 hours) and unmodified liposomes (19.9 hours). Combination of aromatized liposomes and physical encapsulation of hydrophobic ICG into lipid bilayers further extended the duration of action to 73.5 h, a marked improvement over previous TTX formulations³⁹. Aromatized liposomes also drastically mitigated the toxicity and expanded the therapeutic window of TTX, addressing three major challenges in applying neurotoxins like TTX as local anesthetics for clinical use. In current animal models, it is difficult to achieve such long nerve block without sustained drug delivery systems as evidenced by the fact that 5 μg was fatal for rats.

The decreased permeability of aromatized liposomes was attributed to the incorporation of aromatic groups within lipid bilayers. First, n-n stacking interactions between aromatic groups noncovalently crosslinked lipids from opposite layers and adjacent lipids from the same layer, providing additional stabilization forces to rigidify lipid bilayers⁴⁰. Incorporation of phenoxy and coumarin groups increased the Tm of phospholipids from 41.1° C. to 47.4 and 66.1° C., respectively. The coumarin group was slightly less hydrophobic than phenoxy group due to the existence of lactone structure, indicating the additional stabilization of lipid bilayers resulted from n-n stacking interactions instead of hydrophobic interactions. These observations indeed suggested that incorporation of aromatic groups within lipid bilayers was a practical and powerful approach to decrease the permeability of liposomes. The effect of n-n stacking interactions on stabilizing lipid bilayers was also validated by the fact that simply encapsulating aromatic ICG into lipid bilayers also enabled prolonged nerve block in vivo. The effect of aromatic groups modification in stabilizing drug delivery systems and preventing premature drug release was also found in polymeric micelles⁴¹ and hydrogels⁴², indicating broad applicability in applying n-n stacking interactions for sustained drug delivery. Second, terminal aromatic groups on acyl chains likely fill the free space within lipid bilayers and serve as extra physical barriers to slow down the diffusion of hydrophilic molecules⁴³. Thirdly, the presence of aromatic groups is unfavorable for the lateral motion of phospholipids and transient formation of free spaces within lipid bilayers⁴⁴ due to the intrinsic rigidity of ring structures⁴³, which probably decreases the fluidity and permeability of lipid bilayers.

Acute perioperative and chronic pain are among the most prevalent medical conditions⁴⁵. Limited therapeutic alternatives to pain management have produced an overreliance on opioid anesthetics worldwide⁴⁶. Conventional anesthetics are short in duration and the associated neurotoxicity and myotoxicity are severe³⁹. A slight overdose of drugs like fentanyl may lead to severe side effects or even death⁴⁷. TTX and other neurotoxins have emerged as appealing non-opioid alternatives for prolonged pain relief due to their high sodium channel sensitivity, extremely high potency, minimal cardiovascular toxicity, and long-lasting effect²⁶. However, the dosing of these anesthetics is limited because their severe systemic toxicity and narrow therapeutic window¹⁷. This work is of direct relevance to the extensive efforts underway to exploit alternative approaches in addressing the growing epidemic of opioid abuse and overdose. Aromatized liposomes may provide new opportunities for prolonged nonopioid local pain relief.

Aromatized liposomes, as previously described above, may also be attractive for the encapsulation and sustained release of therapeutics with aromatic rings, which account for more than 80% of globally approved drugs⁴⁸. Beyond drug delivery, membrane-modified liposomes could serve as good model systems for the investigation of biological membranes such as structural organizations and the interrelation between different components at the molecular level, which remain largely elusive⁴⁹. The aromatized liposomes can be prepared following existing FDA guidelines with aromatic groups-conjugated phospholipids, which favors their potential clinical translations⁹.

As discussed above, herein is described the improvement of sustained drug release by the aromatization of the inner aspect of liposomal lipid bilayers. Aromatized liposomes increased the drug loading and considerably decreased the release of payloads with different water solubilities and molecular weights. These changes had an impact in vivo: aromatization prolonged the duration of local anesthesia from TTX liposomes to more than 3 days, and curtailed systemic toxicity.

Aromatic groups were incorporated within the lipid bilayers of liposomes to target the rate-limiting step in the passive diffusion of molecules from liposomes. There are a number of mechanisms by which this might occur. n-n stacking interactions, non-covalent crosslinked adjacent phospholipids, decreased lateral motion of lipids within the liposomal membranes, stabilized lipid bilayers, decreased their fluidity and permeability. The rigid ring structures of aromatic groups may reduce the fluidity and permeability of lipid bilayers as observed in some bacteria. The aromatized liposomes may find broad application in the encapsulation and release of a broad range of drugs, including chemotherapeutics, macromolecular drugs, and proteins drugs. The release kinetics suggested the reduced release of small molecules due to aromatization likely correlates to multiple factors. Neither molecular weight nor hydrophilicity correlated strongly with the degree of reduction. The reduced release of macromolecules as result of aromatization correlated with their molecular weight and hydrophilicity.

There were constraints on the effect of aromatization on the loading of liposomes and the resulting release kinetics. While the addition of one ring enhanced performance, a second ring (coumarin) had minimal effect on liposome performance, and greatly increased viscosity—which would adversely affect removal of free drug from the formulation, and injectability in the clinical. Addition of a bulky three-member ring (DBCO) actually impaired performance. That impairment may be due to the bulky group interfering with the packing of hydrocarbon chains and creating free space within lipid bilayers, which could increase the permeability of liposomes. The DBCO group was conjugated to the acyl chain via two amide bonds, which may also make the liposome leakier. It remains to be seen whether further hydrophobic non-bulky modifications of aromatic groups would further enhance liposome performance.

Conventional anesthetics are short in duration and can have associated neurotoxicity and myotoxicity. Limited therapeutic alternatives in pain management have produced an overreliance on opioid anesthetics worldwide. A long-acting formulation of bupivacaine liposome (Exparel) has been approved for clinical use in local anesthesia. However, a recent study revealed that Exparel did not demonstrate significant pain relief compared to standard bupivacaine in 74.58% of randomized clinical trials. Developing new formulations for prolonged non-addictive pain relief has long been of research and clinical interest. Herein is described a formulation that provides three days of nerve block from a single injection. Addition of dexamethasone further extended the duration of analgesia to over a week. One important consideration in that context is that use in larger animals (such as humans) will allow for the use of larger doses, enabling longer blocks. There would also be less systemic toxicity because it tracks relatively linearly with the compound's volume of distribution (i.e., the animal's size) while the duration of nerve block does not. Nerve block lasting 2-3 days would cover the duration of most postoperative pain. Longer durations would be useful for severe localized chronic pain like cancer pain.

In summary, lipid bilayer-engineered vesicles were developed for sustained drug delivery. Incorporation of additional physical barrier and stabilization forces within lipids bilayers increased the drug loading, reduced the burst release of payloads, prolonged the action time, expanded the therapeutic window, and mitigated the systemic toxicity of encapsulated drugs.

### Example 2. Methods for Example 1

### Materials

16-bromohexadecanoic acid, acetyl chloride, anhydrous methanol, phenol, potassium carbonate, anhydrous acetonitrile, anhydrous tetrahydrofuran, hydrochloric acid, 2,6-dichlorobenzoyl chloride, 1-methylimidazole and cholesterol were purchased from Sigma-Aldrich (St. Louis, MO, USA). 16:0 DPPC, 18:1 DOPC, 18:0 DSPG, 16:0 lyso PC, Cy7-DOPC were purchased from Avanti Polar Lipids. Cyanine 7 carboxylic acid was purchased from Lumiprobe Corporation. Tetrodotoxin (TTX) was obtained from Abcam. TTX ELISA kits were purchased from Reagen LLC. Dulbecco's phosphate buffered saline (PBS) were purchased from Thermo Fisher Scientific.

### Synthesis of Aromatic Group-Modified Phospholipids

To a 100 mL round-bottom flask was added 16-bromohexadanoic acid (4 mmol), acetyl chloride (10 mmol) and anhydrous methanol. The solution was stirred overnight at room temperature. After removing of solvent, the resulting solids were resuspended in dichloromethane, then washed with NaHCO₃ (2×200 mL) and brine (2×200 mL). The organic phase was separated, dried over Na₂SO₄ and concentrated to afford the methyl 16-bromohexadecanoate in excellent yield (90-95%). ¹H NMR (CDCl₃, 400 MHz, ppm, δ): 3.66 (s, 3H), 3.44-3.36 (t, 2H), 2.33-2.25 (t, 2H), 1.89-1.79 (m, 2H), 1.66-1.55 (m, 2H), 1.47-1.36 (m, 2H), 1.34-1.21 (m, 20H). ¹³C NMR (CDCl₃, 400 MHz, ppm, δ): 174.09, 51.33, 34.06, 33.78, 32.86, 29.63, 29.61, 29.58, 29.54, 29.45, 29.26, 29.15, 28.78, 28.19, 24.95. ESI-MS: m/z calculated for C₁₇H₃₃BrO₂ [M+H]⁺: 349.2; observed: 349.2.

The methyl 16-bromohexadecanoate (4 mmol) and Na₂CO₃ (10 mmol) was added in 30 mL MeCN in 100 mL round-bottom bottle flask and the solution was heated to 60° C. Phenol (5 mmol) was dissolved in 1 mL MeCN and added to the reaction mixture. The reaction mixture was stirred for 16 hours at 60° C. The reaction mixture was cooled to room temperature, filtrated to remove the salt and concentrated. The crude material was resuspended with in dichloromethane, then washed with NaHCO₃ (2×200 mL) and brine (2×200 mL). The organic phase was separated, dried over Na₂SO₄ and concentrated. The obtained crude materials was purified by silica gel flash chromatography eluting with 10:1 hexanes:ethyl acetate (EtOAc) to afford methyl 16-phenoxyhexadecanoate in good yield (69-75%). ¹H NMR (CDCl₃, 400 MHz, ppm, δ): 7.34-7.22 (m, 2H), 6.96-6.85 (m, 3H), 3.99-3.91 (t, 2H), 3.66 (s, 3H), 2.35-2.25 (t, 2H), 1.83-1.71 (m, 2H), 1.68-1.56 (m, 2H), 1.50-1.39 (m, 2H), 1.39-1.20 (m, 20H). ¹³C NMR (CDCl₃, 400 MHz, ppm, δ): 174.11, 159.28, 129.52, 120.58, 114.66, 68.05, 51.34, 34.21, 29.78, 29.77, 29.72, 29.57, 29.55, 29.45, 29.38, 29.20, 26.21, 24.82. ESI-MS: m/z calculated for C₂₃H₃₈O₃[M+H]⁺: 363.3; observed: 363.3.

To a 100 mL round-bottom flask were added the methyl 16-phenoxyhexadecanoate (4 mmol), THF (20 mL) and deionized water (20 mL). 1 M NaOH (20 mmol) was then added dropwise and reaction mixture was stirred for 16 hours at room temperature. The reaction mixture was diluted with EtOAc (100 mL), washed with 10% HCl (50 mL) washed with deionized water (2×100 mL) and brine (100 mL). The organic phase was separated, dried over Na₂SO₄ and concentrated to afford the 16-phenoxyhexadecanoic acid in good yield (70-80%). ¹H NMR (CDCl₃, 400 MHz, ppm, δ): 7.34-7.22 (m, 2H), 6.96-6.85 (m, 3H), 3.99-3.91 (t, 2H), 2.39-2.30 (t, 2H), 1.84-1.72 (m, 2H), 1.70-1.58 (m, 2H), 1.52-1.41 (m, 2H), 1.39-1.20 (m, 20H). ¹³C NMR (CDCl₃, 400 MHz, ppm, δ): 180.31, 159.28, 129.52, 120.58, 114.66, 68.05, 34.21, 29.78, 29.77, 29.72, 29.57, 29.55, 29.45, 29.38, 29.20, 26.21, 24.82. ESI-MS: m/z calculated for C₁₇H₃₃BrO₂ [M+H]⁺: 349.3; observed: 349.3.

To a solution of 16-phenoxyhexadecanoic acid (1.0 mmol), 16:0 lyso-PC (0.9 mmol) and 1-methylimidazole (3.0 mmol) in CHCl₃ (6 mL) was added 2,6-dichlorobenzoyl chloride (2.0 mmol) and the resulting mixture was stirred for 16 hours at room temperature. The reaction mixture was then concentrated under reduced pressure. The crude was purified by silica gel chromatography (1:1 MeOH/CH₂Cl₂) affording 1-palmitoyl-2-(16-phenoxy) palmitoyl-sn-glycero-3-phosphocholine (Ph-DPPC) as a white wax in moderate yield (50-60%). ¹H NMR (CD₃OD, 400 MHz, ppm, δ): 7.28-7.20 (m, 2H), 6.94-6.85 (m, 3H), 5.29-5.20 (m, 1H), 4.48-4.39 (m, 1H), 4.32-4.22 (m, 2H), 4.21-4.13 (m, 1H), 4.04-3.98 (t, 2H), 3.97-3.92 (t, 2H), 3.66-3.61 (t, 2H), 3.22 (s, 9H), 2.38-2.28 (m, 4H), 1.80-1.72 (m, 2H), 1.66-1.55 (m, 4H), 1.52-1.42 (m, 4H), 1.41-1.22 (m, 44H) 0.93-0.86 (t, 3H). ¹³C NMR (CDCl₃, 400 MHz, ppm, δ): 174.93, 173.62, 160.61, 130.39, 121.49, 115.53, 71.79, 68.88, 67.50, 64.92, 63.69, 60.49, 54.75, 35.12, 34.94, 33.08, 30.81, 30.78, 30.71, 30.66, 30.52, 30.49, 30.46, 30.45, 30.23, 30.20, 27.19, 26.05, 26.03, 23.74. ESI-MS: m/z calculated for C₄₆H₈₄NO₉P [M+H]⁺: 826.6; observed: 826.6.

### Liposome Preparation and Characterization

The liposomes were prepared using the thin-film hydration method. The lipid formulation (Ph-DPPC, DOPC, DSPG and cholesterol at a molar ratio of 3:3:2:3) was dissolved in a solution of chloroform and methanol (ratio: 9:1). The solvent was evaporated under reduced pressure, and the lipid was redissolved in tert-butanol, followed by freeze-drying. The lipid cake was hydrated with SRho, Bup, TTX, ICG, polyethylene glycol, FITC-Albumin in PBS buffer (pH=7.4) or hydrated with bupivacaine hydrochloride, or doxorubicin hydrochloride in saline. After ten freeze-thaw cycles, the solution was dialyzed against PBS for 48 hours in a dialysis tube with a molecular mass cut-off of 1,000 kDa. The dialysis media were changed with fresh PBS at least twice a day. Drugs and dye in all formulations were quantitated after disruption of the liposome with octyl-β-D-glucopyranoside (100 mM, volume ratio of 2:1 to formulations).

### Instruments and Characterization of Materials

¹H and ¹³C NMR experiments were measured on a Varian 400 M NMR spectrometer. Dynamic light scattering (DLS) (Zetasizer Pro; Malvern Panalytical, USA) was used to determine the hydrodynamic diameter and zeta potential of liposomes. The liposome solutions were also characterized by transmission electron microscopy (TEM, Tecnai G2 T20; FEI company, OR, USA) using a negative staining method with uranyl acetate (1.0% w/w). An Agilent 1260 series high-performance liquid chromatography (HPLC) with a UV-vis detector was used for analyzing the Bupivacaine hydrochloride in the releasing experiments. An Agilent Infinity lab LC/MSD XT single quadrupole mass analyzer was used for molecular weight measurement. Viscosity was tested on a TA DHR-2 rheometer with a frequency of 1 Hz, shear strain of 1%, and a temperature of 25° C.

### Partition Coefficient Quantification

Octanol-water partition coefficients (Log P) were quantified using a miniaturized shake-flask approach. To investigate the analytes in either fully ionized or neutral state as well as their pH-dependent partitioning behavior, measurements were performed using sodium-based buffer solutions at a constant ionic strength of 0.1 M covering three different pH values: pH 4.0 (citrate buffer), pH 7.4 (phosphate buffer), and pH 10.2 (carbonate buffer). All buffer solutions were saturated with octanol prior to analysis and vice versa. All octanol-water mixtures were vortexed for 5 min and then stirred for 24 h at room temperature to reach equilibrium and phase distribution. Kinetic measurements were performed to confirm the equilibrium conditions in the setup. After equilibration and phase separation, all samples were analyzed on an Agilent HPLC 1260 or a plate reader (BioTek, Winooski, VT).

As described in the liposome preparation section, the lipid cake was hydrated with SRho, TTX, polyethylene glycol (PEG), FITC-albumin in PBS buffer (pH=7.4) or hydrated with bupivacaine hydrochloride, doxorubicin hydrochloride in saline. The pH of bupivacaine hydrochloride and doxorubicin hydrochloride in physiological saline at the concentration tested is close to 4 as measured by a Mettler Toledo Seven Easy pH meter. Thus, Log P values of bupivacaine hydrochloride and doxorubicin hydrocholoride at pH 4.0 were used for the comparison of loading and release. Log P values at pH 7.4 of other payloads were used for the comparison of loading and release.

### Cell Culture

Cell culture of C2C12 mouse myoblasts (American Type Culture Collection (ATCC), Manassas, VA, USA) and PC12 rat adrenal gland pheochromocytoma cells (ATCC, Manassas, VA, USA) were performed. C2C12 cells were cultured in DMEM with 20% FBS and 1% Penicillin Streptomycin. Cells were seeded onto a 24-well plate at 50,000 cells mL-1 and incubated for 10-14 days in DMEM with 2% horse serum and 1% Penicillin Streptomycin to differentiate into myotubules. PC12 cells were grown in DMEM with 12.5% horse serum, 2.5% FBS, and 1% Penicillin Streptomycin. Cells were seeded onto a 24-well-plate, and 50 ng mL-1 nerve growth factor was added 24 hours after seeding.

### Cell Viability

To determine the cytotoxicity, cells were exposed to different TTX formulations using a 24-well Transwell® membrane system (Costar 3495, pore size 0.4 m) (Corning Incorporated, ME, USA). Cells were incubated in 0.9 mL of media in the cell culture wells, and 100 μL of test samples were added above the Transwell® membranes, which were immersed in the media in the wells. Cell viability was evaluated by the MTS assay (Promega, WI, USA) 96 hours after incubation.

Cytotoxicity of the materials to muscle and nerve cells was also assessed by a direct contact setup. Different formulations were directly added into the cell culture media and incubated in the media bathing the cells (i.e., in direct contact with them) in conventional cell culture wells (lipid concentration: 20 mg/mL). After 24 hours, cell viabilities were evaluated with the MTS assay, and their survival expressed as percentages of results in untreated cells.

### In Vitro Drug Release

Cumulative release of small molecules (e.g., Sulforhodamine B, Tetrodotoxin, Bupivacaine hydrochloride, and doxorubicin hydrochloride) were performed by placing 200 L of samples into a Slide-A-Lyzer MINI dialysis device (Thermo Fisher Scientific, Waltham, MA) with a 10,000 MW cut-off, further dialyzed with 14 mL release media and incubated at 37° C. on a platform shaker (New Brunswick Innova 40, 60 rpm). At predetermined intervals, the dialysis solution was exchanged with fresh, pre-warmed release media. The release media of Sulforhodamine B, Tetrodotoxin was PBS (pH=7.4). The release media of Bupivacaine hydrochloride, and doxorubicin hydrochloride was physiological saline. The concentration of TTX in release media was quantified by an enzyme-linked immunosorbent assay (ELISA, Reagen LLC). The concentration of Sulforhodamine B in release media was determined by a plate reader (BioTek, Winooski, VT) with excitation and emission wavelengths of 560 nm and 580 nm. The concentration of bupivacaine hydrochloride (Bup) in release media was determined by high-performance liquid chromatography. The concentrations of SRho, TTX and Bup in release studies were 4 mM, 0.3 mM and 20 mM as reported previously.

The maximum cut-off of commercially available Slide-A-Lyzer MINI dialysis device was 20,000 MW. Thus, the cumulative release of macromolecules was performed by placing 500 μL of samples into a Float-A-Lyzer G2 dialysis devices (Spectrum Laboratories Inc, Piscataway, NJ) with a 1000,000 MW cut-off, further dialyzed with 14 mL PBS and incubated at 37° C. on a platform shaker (New Brunswick Innova 40, 60 rpm). At predetermined intervals, the dialysis solution was exchanged with fresh, pre-warmed release media. The concentration of dye-conjugated macromolecules was determined by a plate reader (BioTek, Winooski, VT). The concentrations of SRho-PEG1k, SRho-PEG10k and FITC-Ab in release studies were 1 mM, 0.5 mM and 0.5 mM, respectively.

### Animal Studies

Animal studies were conducted following protocols approved by the Boston Children's Hospital Animal Care and Use Committee in accordance with the guidelines of the International Association for the Study of Pain. Adult male Sprague-Dawley rats (Charles River Laboratories, Wilmington, MA, USA) weighing 350-400 g were housed in groups under a 12-hours/12-hours light/dark cycle with lights on at 6:00 AM.

Sciatic nerve injections were performed with a 23 G needle at the left sciatic nerve under brief isoflurane-oxygen anesthesia. The needle was introduced posteromedial to the greater trochanter, pointing in the anteromedial direction, and upon contact with bone, the formulations were injected onto the sciatic nerve.

Neurobehavioural testing was conducted on both hindquarters. Deficits in the right (uninjected) extremity served as a metric of systemic drug distribution. Sensory nerve blockade was assessed by modified hotplate testing. The hind paws were exposed in sequence (left then right) to a 56° C. hot plate (Stoelting, Wood Dale, IL, USA), and the time the animal allowed its paw to remain on the hotplate (thermal latency) was measured. A thermal latency of 2 seconds indicated no nerve blockade (baseline), and a thermal latency of 12 seconds was maximal latency. Successful nerve blockade was defined as achieving a thermal latency above 7 seconds. Hind paws were removed from the hotplate after 12 seconds to prevent thermal injury. Measurements were repeated three times in each animal at each time point and the median was used for further data analysis.

Motor nerve block was assessed by a weight-bearing test to determine the motor strength of the rat's hind paw. The rat was positioned with one hind paw on a digital balance and was allowed to bear its own weight. The maximum weight that the rat could bear without the ankle touching the balance was recorded, and motor block was considered achieved when the motor strength was less than half maximal. Measurements were repeated three times at each time point and the median was used for further data analysis.

Duration of sensory block were calculated as the time required for thermal latency to return to 7 seconds (halfway between the baseline and maximal latencies). The duration of motor block was defined as the time it took for the weight-bearing to return to halfway between normal and maximal block.

### Laser Scanning Confocal Microscopy (LSCM) Imaging

Cy7-conjugated DOPC was used in the preparation of Cy7-labeled liposomes following the same procedures as described previously with Cy7 molar concentration at 0.2 mg/mL. Under brief isoflurane-oxygen anesthesia, rats were injected with 0.3 mL of different Cy7 labeled liposomal formulations. Sciatic nerves together with surrounding tissues were harvested and embedded into OCT compound (VWR, PA, USA), then frozen and stored at −20° C. Sections (10 m) were prepared using a cryostat microtome (Leica CM3050 S, Wetzlar, Germany) and mounted onto glass slides. Afterward, slides were fixed with 4% paraformaldehyde for 20 minutes at room temperature, washed in PBS (pH 7.4) 3 times. Nuclei were stained with Hoechst 33342. The slices were imaged using a Zeiss LSM 710 multi-photon confocal microscopy (Carl Zeiss AG, Oberkochen, Germany).

### In Vivo Imaging System (IVIS) Imaging

Cy7-conjugated DOPC was used in the preparation of Cy7-labeled liposomes following the same procedures as described previously with Cy7 molar concentration at 0.2 mg/mL. Under isoflurane-oxygen anesthesia, rats were shaved and injected with 0.3 mL of different Cy7-labeled liposomal formulation. The in vivo fluorescence images were captured, and the fluorescence intensity was evaluated at predetermined time points post-injection (under brief isoflurane-oxygen anesthesia) using a Spectrum IVIS (PerkinElmer, MA, USA). Whole-body animal images were recorded non-invasively. The 745 nm excitation filter and the 800 nm emission filter were used for the imaging. Quantitative analysis was carried out using the Live Imaging® software of the IVIS.

### Tissue Harvesting and Histology

Rats were sacrificed at 4 days and 14 days after the injection (it was determined that these time points were useful in evaluating both acute and chronic inflammation and myotoxicity), and the sciatic nerve was harvested together with surrounding tissues. The samples were scored for inflammation (0-4) and myotoxicity (0-6). All scoring and other histological assessments were performed by an observer blinded as to the nature of the individual samples.

The inflammation score was a subjective quantification of severity in which 0 was normal and 4 was severe inflammation. The myotoxicity score was determined by the nuclear internalization and regeneration of myocytes, two representative characteristics of local anesthetics' myotoxicity. Nuclear internalization was characterized by myocytes having nuclei located away from their usual location at the periphery of the cell. Regeneration was characterized by the presence of shrunken myocytes with basophilic cytoplasm. The scoring scale was as follows: 0=normal; 1=perifascicular internalization; 2=deep internalization (more than five cell layers); 3=perifascicular regeneration; 4=deep tissue regeneration (more than five cell layers); 5=hemifascicular regeneration; 6=holofascicular regeneration.

To evaluate the neurotoxicity of formulations, the sciatic nerves were fixed in Karnovsky's KII solution, processed and Epon-embedded for toluidine blue staining. They were assessed by optical microscopy in a masked fashion.

### Statistical Analysis.

Statistical comparisons were performed using the Student t-test (one-sided) unless stated otherwise. Thermal latency, inflammation and myotoxicity scores were reported as medians and quartiles due to their ordinal or non-Gaussian character. Data are presented as means±SD (n=4) in release kinetics, cell work, neurobehavioral, and histology studies. Data were considered statistically significant if P<0.05 (****P<0.0001, ***P<0.001, **P<0.01, *P<0.05).

### REFERENCES

1. Torchilin, V. P. Recent advances with liposomes as pharmaceutical carriers. Nature reviews Drug discovery 4, 145 (2005).
2. Bulbake, U., Doppalapudi, S., Kommineni, N. & Khan, W. Liposomal formulations in clinical use: an updated review. Pharmaceutics 9, 12 (2017).
3. Xing, H., Hwang, K. & Lu, Y. Recent developments of liposomes as nanocarriers for theranostic applications. Theranostics 6, 1336 (2016).
4. Allen, T. M. & Cullis, P. R. Liposomal drug delivery systems: from concept to clinical applications. Advanced drug delivery reviews 65, 36-48 (2013).
5. Bozzuto, G. & Molinari, A. Liposomes as nanomedical devices. International journal of nanomedicine 10, 975 (2015).
6. Barenholz, Y. Liposome application: problems and prospects. Current opinion in colloid & interface science 6, 66-77 (2001).
7. Kakkar, A., Traverso, G., Farokhzad, O. C., Weissleder, R. & Langer, R. Evolution of macromolecular complexity in drug delivery systems. Nature Reviews Chemistry 1, 1-17 (2017).
8. Papahadjopoulos, D., Nir, S. & Ohki, S. Permeability properties of phospholipid membranes: effect of cholesterol and temperature. Biochimica et Biophysica Acta (BBA)-Biomembranes 266, 561-583 (1972).
9. Pattni, B. S., Chupin, V. V. & Torchilin, V. P. New developments in liposomal drug delivery. Chemical reviews 115, 10938-10966 (2015).
10. Moon, J. J., et al. Interbilayer-crosslinked multilamellar vesicles as synthetic vaccines for potent humoral and cellular immune responses. Nature materials 10, 243 (2011).
11. Storck, E. M., Ozbalci, C. & Eggert, U. S. Lipid cell biology: a focus on lipids in cell division. Annual review of biochemistry 87, 839-869 (2018).
12. Harayama, T. & Riezman, H. Understanding the diversity of membrane lipid composition. Nature reviews Molecular cell biology 19, 281 (2018).
13. Yang, N. J. & Hinner, M. J. Getting across the cell membrane: an overview for small molecules, peptides, and proteins. in Site-specific protein labeling 29-53 (Springer, 2015).
14. Lodish, H., et al. Diffusion of small molecules across phospholipid bilayers. Molecular Cell Biology (2000).
15. Barba-Bon, A., Nilam, M. & Hennig, A. Supramolecular chemistry in the biomembrane. ChemBioChem (2020).
16. Sezgin, E., Levental, I., Mayor, S. & Eggeling, C. The mystery of membrane organization: composition, regulation and roles of lipid rafts. Nature reviews Molecular cell biology 18, 361 (2017).
17. Martinez, C. R. & Iverson, B. L. Rethinking the term “pi-stacking”. Chemical Science 3, 2191-2201 (2012).
18. Krieg, E., Bastings, M. M., Besenius, P. & Rybtchinski, B. Supramolecular polymers in aqueous media. Chemical reviews 116, 2414-2477 (2016).
19. Deng, J.-H., et al. π-π stacking interactions: Non-negligible forces for stabilizing porous supramolecular frameworks. Science advances 6, eaax9976 (2020).
20. Zhuang, W.-R., et al. Applications of π-π stacking interactions in the design of drug-delivery systems. Journal of Controlled Release 294, 311-326 (2019).
21. Morin, C., Simon, D. & Sautet, P. Chemisorption of benzene on Pt (111), Pd (111), and Rh (111) metal surfaces: a structural and vibrational comparison from first principles. The Journal of Physical Chemistry B 108, 5653-5665 (2004).
22. Li, L., Vorobyov, I. & Allen, T. W. The different interactions of lysine and arginine side chains with lipid membranes. The Journal of Physical Chemistry B 117, 11906-11920 (2013).
23. Acharya, H. P. & Kobayashi, Y. A rapid condensation between lysophosphorylcholine and fatty acids with an easily separable amine base. Synlett 2005, 2015-2018 (2005).
24. Rwei, A. Y., et al. Ultrasound-triggered local anaesthesia. Nature biomedical engineering 1, 644-653 (2017).
25. Lahaye, L. A. & Butterworth, J. F. Site-1 Sodium Channel Blockers as Local AnestheticsWill Neosaxitoxin Supplant the Need for Continuous Nerve Blocks?Anesthesiology: The Journal of the American Society of Anesthesiologists 123, 741-742 (2015).
26. Kohane, D. S., et al. A re-examination of tetrodotoxin for prolonged duration local anesthesia. Anesthesiology: The Journal of the American Society of Anesthesiologists 89, 119-131 (1998).
27. Kohane, D. S., et al. Prolonged duration local anesthesia from tetrodotoxin-enhanced local anesthetic microspheres. Pain 104, 415-421 (2003).
28. Bane, V., Lehane, M., Dikshit, M., O'Riordan, A. & Furey, A. Tetrodotoxin: Chemistry, toxicity, source, distribution and detection. Toxins 6, 693-755 (2014).
29. Lomonte, B., et al. Comparative study of the cytolytic activity of myotoxic phospholipases A2 on mouse endothelial (tEnd) and skeletal muscle (C2C12) cells in vitro. Toxicon 37, 145-158 (1999).
30. Slotkin, T. A., MacKillop, E. A., Ryde, I. T., Tate, C. A. & Seidler, F. J. Screening for developmental neurotoxicity using PC12 cells: comparisons of organophosphates with a carbamate, an organochlorine, and divalent nickel. Environmental health perspectives 115, 93-101 (2007).
31. Kohane, D. S. Microparticles and nanoparticles for drug delivery. Biotechnology and bioengineering 96, 203-209 (2007).
32. McAlvin, J. B., et al. Multivesicular liposomal bupivacaine at the sciatic nerve. Biomaterials 35, 4557-4564 (2014).
33. Wang, Y.-W., et al. Dye-enhanced graphene oxide for photothermal therapy and photoacoustic imaging. Journal of Materials Chemistry B 1, 5762-5767 (2013).
34. Reinhart, M. B., Huntington, C. R., Blair, L. J., Heniford, B. T. & Augenstein, V. A. Indocyanine green: historical context, current applications, and future considerations. Surgical innovation 23, 166-175 (2016).
35. Neal, J. M., Salinas, F. V. & Choi, D. S. Local anesthetic-induced myotoxicity after continuous adductor canal block. Regional Anesthesia & Pain Medicine 41, 723-727 (2016).
36. Jia, X., Colombo, G., Padera, R., Langer, R. & Kohane, D. S. Prolongation of sciatic nerve blockade by in situ cross-linked hyaluronic acid. Biomaterials 25, 4797-4804 (2004).
37. Padera, R. F., Tse, J. Y., Bellas, E. & Kohane, D. S. Tetrodotoxin for prolonged local anesthesia with minimal myotoxicity. Muscle & Nerve: Official Journal of the American Association of Electrodiagnostic Medicine 34, 747-753 (2006).
38. Tibbitt, M. W., Dahlman, J. E. & Langer, R. Emerging frontiers in drug delivery. Journal of the American Chemical Society 138, 704-717 (2016).
39. Santamaria, C. M., Woodruff, A., Yang, R. & Kohane, D. S. Drug delivery systems for prolonged duration local anesthesia. Materials Today 20, 22-31 (2017).
40. Fleming, S. & Ulijn, R. V. Design of nanostructures based on aromatic peptide amphiphiles. Chemical Society Reviews 43, 8150-8177 (2014).
41. Shi, Y., et al. Complete regression of xenograft tumors upon targeted delivery of paclitaxel via H—H stacking stabilized polymeric micelles. ACS nano 9, 3740-3752 (2015).
42. Li, F., et al. Smart Hydrogels Co-switched by Hydrogen Bonds and n-R Stacking for Continuously Regulated Controlled-Release System. Advanced Functional Materials 20, 669-676 (2010).
43. Monteiro, N., Martins, A., Reis, R. L. & Neves, N. M. Liposomes in tissue engineering and regenerative medicine. Journal of the Royal Society Interface 11, 20140459 (2014).
44. Vaz, W. L., Goodsaid-Zalduondo, F. & Jacobson, K. Lateral diffusion of lipids and proteins in bilayer membranes. FEBS letters 174, 199-207 (1984).
45. Wu, C. L. & Raja, S. N. Treatment of acute postoperative pain. The Lancet 377, 2215-2225 (2011).
46. Califf, R. M., Woodcock, J. & Ostroff, S. A proactive response to prescription opioid abuse. New England Journal of Medicine 374, 1480-1485 (2016).
47. Schmid, C. L., et al. Bias factor and therapeutic window correlate to predict safer opioid analgesics. Cell 171, 1165-1175. e1113 (2017).
48. Mao, F., et al. Chemical structure-related drug-like criteria of global approved drugs. Molecules 21, 75 (2016).
49. Siontorou, C. G., Nikoleli, G.-P., Nikolelis, D. P. & Karapetis, S. K. Artificial lipid membranes: past, present, and future. Membranes 7, 38 (2017).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects described herein, is/are referred to as comprising particular elements and/or features, certain embodiments described herein or aspects described herein consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments described herein, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment described herein can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A composition comprising:
a compound of Formula (I): 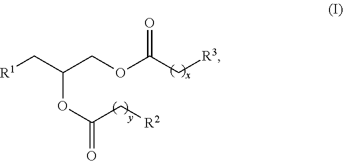
or a salt, co-crystal, tautomer, stereoisomer, solvate, hydrate, polymorph, or an isotopically enriched derivative thereof, wherein:
x is 1, 2, 3, 4, 5, 6, 7, 8, 9, 10, 11, 12, 13, 14, 15, 16, 17, 18, 19, 20, 21, 22, 23, or 24;
y is 1,2,3,4,5,6,7,8,9,10,11,12,13,14,15,16,17,18, 19,20,21,22,23, or 24;
R¹ is a phosphoglycerol, phosphocholine, phosphoethanolamine, phosphoinositol, or phosphoserine moiety;
R² is halogen, optionally substituted acyl, optionally substituted branched or unbranched alkyl, optionally substituted carbocyclyl, optionally substituted heterocyclyl, optionally substituted aryl, —OR^{D1}, —N(R^{D1a})₂, —SR^{D1}, SO₂, —NO₂, —N₃, or —CN;
R³ is halogen, optionally substituted acyl, optionally substituted branched or unbranched alkyl, optionally substituted carbocyclyl, optionally substituted heterocyclyl, optionally substituted aryl, —OR^{D1}, —N(R^{D1a})₂, —SR^{D1}, SO₂, —NO₂, —N₃, or —CN;
wherein R^{D1} is independently hydrogen, optionally substituted acyl, optionally substituted alkyl, optionally substituted alkenyl, optionally substituted cycloalkynyl group, optionally substituted carbocyclyl, optionally substituted heterocyclyl, optionally substituted aryl, optionally substituted heteroaryl, or an oxygen protecting group when attached to an oxygen atom, or a sulfur protecting group when attached to a sulfur atom; and
wherein each occurrence of R^{D1a} is independently hydrogen, optionally substituted acyl, optionally substituted alkyl, optionally substituted alkenyl, optionally substituted linear alkynyl, optionally substituted cycloalkynyl group, optionally substituted carbocyclyl, optionally substituted heterocyclyl, optionally substituted aryl, optionally substituted heteroaryl, or a nitrogen protecting group; or optionally two instances of R^{D1a} are taken together with their intervening atoms to form a substituted or unsubstituted heterocyclic or substituted or unsubstituted heteroaryl ring.

2. The composition of claim 1, wherein the compound is of Formula (I-A): 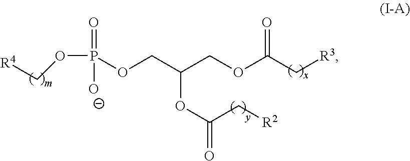
or a salt, solvate, hydrate, stereoisomer, polymorph, tautomer, isotopically enriched form, or prodrug thereof, wherein:
R⁴ is 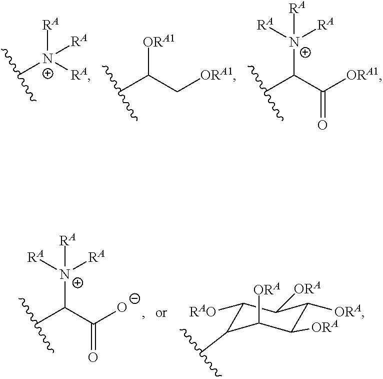
as valency permits;
each occurrence of R^{A} is independently hydrogen or unsubstituted alkyl;
each occurrence of R^{A1} is independently hydrogen, unsubstituted alkyl, or an oxygen protecting group; and
m is 1, 2, 3, 4, 5, or 6.

3. The composition of claim 1 or 2, wherein the compound is of Formula (I-A-1): 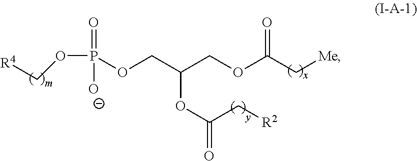
or a salt, solvate, hydrate, stereoisomer, polymorph, tautomer, isotopically enriched form, or prodrug thereof, wherein:
R⁴ is 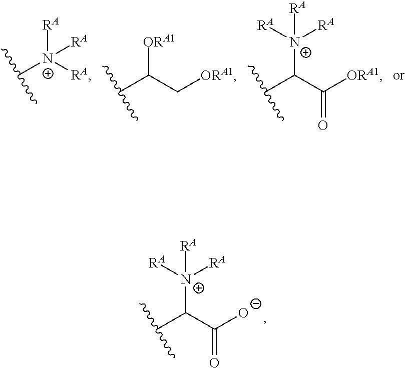
as valency permits;
each occurrence of R^{A} is independently hydrogen or unsubstituted alkyl;
each occurrence of R^{A1} is independently hydrogen, unsubstituted alkyl, or an oxygen protecting group; and
m is 1, 2, 3, 4, 5, or 6.

4. The composition of claim 1, wherein R¹ is an unsubstituted phosphoglycerol, unsubstituted phosphocholine, unsubstituted phosphoethanolamine, unsubstituted phosphoinositol, or unsubstituted phosphoserine moiety.

5. The composition of claim any one of claims 2-4, wherein R⁴ is

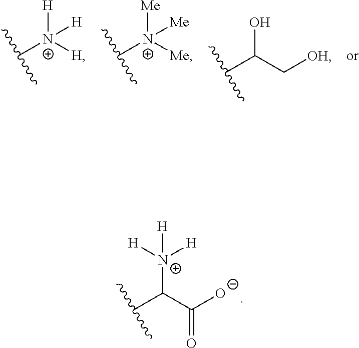

6. The composition of claim any one of claims 2-5, wherein R⁴ is

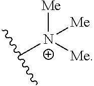 [SMILES: *[N+](C)(C)C]

7. The composition of any one of claims 2-6, wherein m is 2.

8. The composition of any one of claims 1-7, wherein x is 12, 13, 14, 15, or 16.

9. The composition of any one of claims 1-8, wherein x is 14.

10. The composition of any one of claims 1-9, wherein y is 15.

11. The composition of any one of claims 1-10, wherein R² is —OR^{D1}, and R^{D1} is optionally substituted acyl, optionally substituted aryl, optionally substituted heteroaryl, optionally substituted cycloalkynyl group, or optionally substituted linear alkynyl.

12. The composition of any one of claims 1-11, wherein R³ is —OR^{D1}, and R^{D1} is optionally substituted acyl, optionally substituted aryl, optionally substituted heteroaryl, optionally substituted cycloalkynyl group, or optionally substituted linear alkynyl.

13. The composition of any one of claims 1-11, wherein R³ is methyl and R² is —OR^{D1} and R^{D1} is optionally substituted acyl, optionally substituted aryl, optionally substituted heteroaryl, optionally substituted cycloalkynyl group, or optionally substituted linear alkynyl.

14. The composition of any one of claims 1-13, wherein R^{D1} is 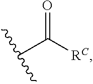
optionally substituted linear alkynyl, optionally substituted phenyl, optionally substituted naphthyl, optionally substituted anthryl, optionally substituted 2H-chromen-2-one (coumarin), optionally substituted cyclooctynyl, optionally substituted dibenzocyclooctyne, or optionally substituted aza-dibenzocyclooctyne; and
R^{C} is hydrogen, optionally substituted alkyl, or optionally substituted alkenyl.

15. The composition of any one of claims 1-14, wherein R² is of the formula:

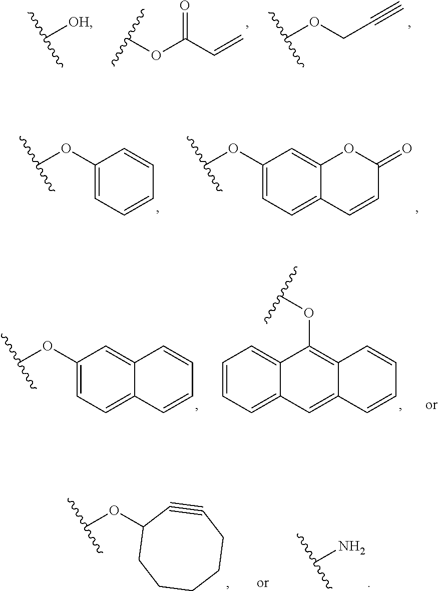

16. The composition of any one of claims 1-10, wherein:
R² is —N(R^{D1a})₂, —N(R^{D1a})C(═O)OR^{D1}, or —SR^{D1};
R^{D1} is hydrogen, optionally substituted alkyl, optionally substituted aryl, optionally substituted heteroaryl, or optionally substituted cycloalkynyl group; and
each instance of R^{D1a} is independently hydrogen, optionally substituted acyl, optionally substituted alkyl, or optionally substituted alkenyl.

17. The composition of claim 16, wherein R² is of the formula: 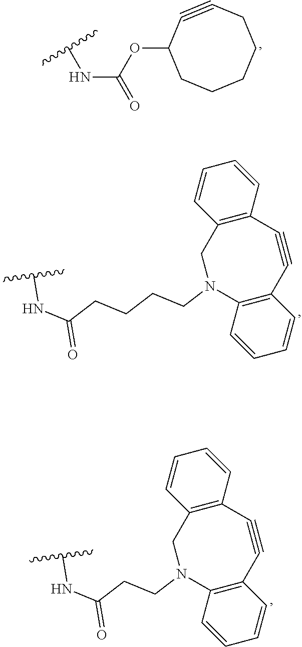 [SMILES: *NC(=O)OC1C#CCCCCC1 ; *NC(=O)CCCCN1Cc2ccccc2C#Cc2ccccc21 ; *NC(=O)CCN1Cc2ccccc2C#Cc2ccccc21]
or —SH.

18. The composition of any one of claims 1-10, wherein R² is —Br or —N₃.

19. The composition of any one of claims 1-14, wherein R² is of the formula: 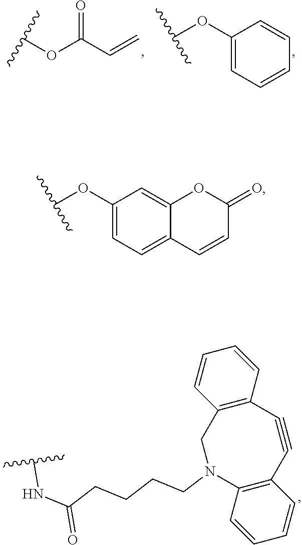
—SH, or —N₃.

20. The composition of any one of claims 1-14, wherein R² is

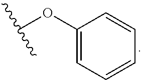 [SMILES: *Oc1ccccc1]

21. The composition of any one of claims 1-20, wherein R³ is optionally substituted linear alkyl.

22. The composition of any one of claims 1-21, wherein R³ is optionally substituted linear C₁₋₄ alkyl.

23. The composition of any one of claims 1-22, wherein R³ is methyl.

24. The composition of any one of claims 1-20, wherein R³ is methyl or —N₃.

25. The composition of any one of claims 1-20, wherein R³ is of the formula:

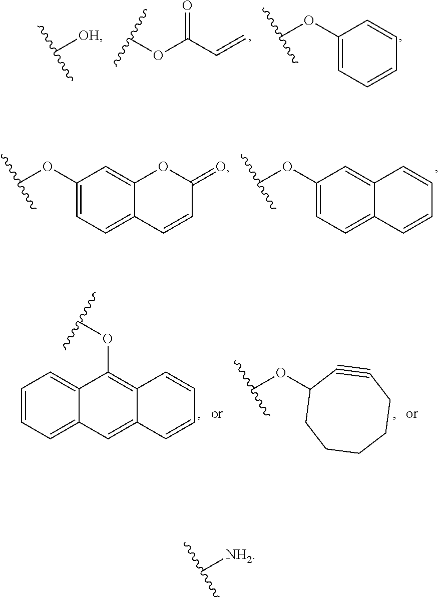

26. The composition of any one of claims 1-20, wherein R³ is —Br or —N₃.

27. The composition of any one of claims 1-26, wherein the compound is of formula: 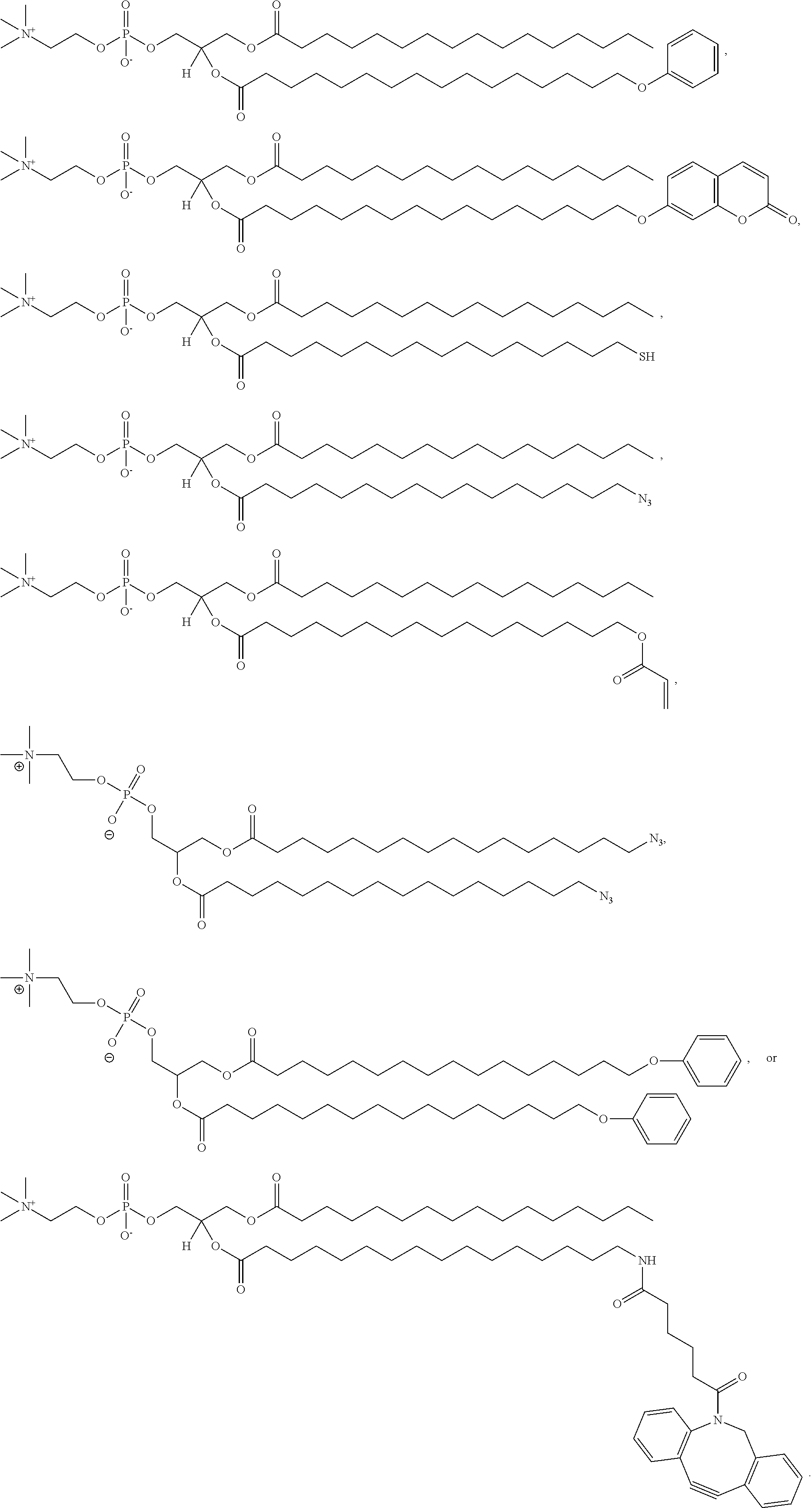
or a salt, co-crystal, tautomer, stereoisomer, solvate, hydrate, polymorph, or an isotopically enriched derivative thereof.

28. The composition of any one of claims 1-27, further comprising one or more agents.

29. The composition of claim 28, wherein at least one of the one or more agents is a therapeutic agent or diagnostic agent.

30. The composition of claim 28 or 29, wherein the therapeutic agent is an antibiotic agent, chemotherapeutic agent, anesthetic agent, anti-inflammatory agent, analgesic agent, anti-fibrotic agent, anti-sclerotic agent, or anticoagulant agent.

31. The composition of claim 29 or 30, wherein the therapeutic agent is a local anesthetic.

32. The composition of claim 31, wherein the local anesthetic is a site 1 sodium channel blocker, amino ester local anesthetic, or an amino amide local anesthetic.

33. The composition of any one of claims 29-32, wherein the therapeutic agent is doxorubicin, tetrodotoxin, saxitoxin, neosaxitoxin, bupivacaine, amylocaine, ambucaine, articaine, benzocaine, benzonatate, butacaine, butanilicaine, carbocaine, cepastat, chloraseptic, chloroprocaine, cinchocaine, citanest, cyclomethycaine, dibucaine, diperodon, dimethocaine, eucaine, etidocaine, fomocaine, fotocaine, hydroxyprocaine, isobucaine, levobupivacaine, lidocaine, marcaine, mepivacaine, meprylcaine, metabutoxycaine, nitracaine, orthocaine, orabloc, oxetacaine, oxybuprocaine, paraethoxycaine, phenacaine, piperocaine, piridocaine, polocaine, posimir, pramocaine, prilocaine, primacaine, procaine, procainamide, proparacaine, propoxycaine, pyrrocaine, quinisocaine, ropivacaine, sensorcaine, septocaine, trimecaine, tetracaine, tolycaine, tropacocaine, ulcerease, xylocaine, or zorcaine.

34. The composition of any one of claims 29-33, wherein the therapeutic agent is doxorubicin, tetrodotoxin, or bupivacaine.

35. The composition of any one of claims 28-32, wherein the agent is a small molecule therapeutic agent, a protein therapeutic agent, a nucleic acid therapeutic agent, or small molecule diagnostic agent.

36. The composition of claim 35, wherein the diagnostic agent is a fluorophore.

37. The composition of claim 35 or 36, wherein the diagnostic agent is conjugated to a protein, a polymer, or a small molecule.

38. The composition of any one of claims 33-37, wherein the diagnostic agent is Sulforhodamine B, indocyanine green, fluorescein isothiocyanate, methylene blue, or coumarin.

39. The composition of any one of claims 28-38, comprising two types of therapeutic agents, wherein the therapeutic agents are selected from the group consisting of a local anesthetic, an anti-inflammatory agent, and a sympathomimetic agent.

40. The composition of any one of claims 1-39, wherein the composition comprises dexamethasone and/or epinephrine.

41. The composition of any one of claims 1-40, wherein the composition is in the form of a particle.

42. The composition of any one of claims 1-41, wherein the particle is a liposome, lipid nanoparticle, polymer-lipid hybrid nanoparticle, lipid micelle, micelle-hydrogel hybrid, liposome-entrapped hydrogel, lipid-peptide complex, lipid-nucleic acid complex, lipid-peptide-nucleic acid complex, or lipid coated inorganic nanoparticle.

43. The composition of claim 41 or 42 wherein the composition comprises dexamethasone in the particle; and epinephrine in an injectate.

44. A pharmaceutical composition comprising a composition of any one of claims 1-43, and optionally a pharmaceutically acceptable excipient.

45. A pharmaceutical composition comprising a composition of any one of claims 1-43, a therapeutic agent, and optionally a pharmaceutically acceptable excipient.

46. A method of delivering an agent to a subject or biological sample, comprising administering to the subject or contacting the biological sample with a composition according to any one of claims 28-43, or administering to the subject or contacting the biological sample with the pharmaceutical composition of claim 44 or 45.

47. A method of treating and/or preventing a disease in a subject in need thereof, the method comprising administering to the subject a composition according to any one of claims 28-43 comprising a therapeutically effective amount of a therapeutic agent, or a pharmaceutical composition of claim 44 or 45.

48. Use of a composition delivering an agent to a subject, the use comprising administering to the subject a composition of any one of claims 28-45.

49. Use of a composition to treat and/or prevent a disease in a subject in need thereof, the use comprising administering to the subject a composition according to any one of claims 28-43 comprising a therapeutically effective amount of a therapeutic agent, or a pharmaceutical composition according to claim 44 or 45.

50. A kit for delivering an agent to a subject, comprising a composition of any one of claims 28-45, the agent, and instructions for delivering the agent to a subject in need thereof.
